(19) United States
(12) Patent Application Publication
(10) Pub. No.: US 2020/0337314 A1
(43) Pub. Date: Oct. 29, 2020

(54) MICROBIAL-BASED COMPOSITION AND METHOD OF USE

(71) Applicant: Sustainable Community Development, LLC, Kansas City, MO (US)

(72) Inventors: Matthew T. Wood, Kansas City, MO (US); Narin Tipsrisukond, Kansas City, MO (US); Amanda Kramer, Oak Ridge, TN (US); Daoxin Li, Kansas City, MO (US)

(21) Appl. No.: 16/958,515

(22) PCT Filed: Dec. 28, 2018
(86) PCT No.: PCT/US2018/068033

§ 371 Date: Jun. 26, 2020

(51) Int. Cl. A01N 63/20; C05F 11/08; C05F 17/20; C05G 5/20; A01N 63/22; A01N 63/30; A01N 63/38

(52) U.S. Cl. CPC A01N 63/20; C05F 11/08; C05F 17/20; C05G 5/20; C12N 2500/76; A01N 63/30; A01N 63/38; C12N 2502/00; A01N 63/22

(57) ABSTRACT

A microbial-based composition provides a co-cultured microorganism consortium in culture medium that includes one or more Acetobacter sp., Bacillus sp., Bifidobacterium sp., Enter ococus sp., Gluconacetobacter sp., Lactobacillus sp., Rhodopseudomonas sp., Saccharomyces sp., Pichia sp., and Trichoderma sp., as well as a carbon source and chlorine-free water. In some embodiments, the microbial-based composition is useful in the agricultural industry as a plant growth promoting and silage-enhancing agent. For plant growth promotion applications, the microbial-based composition may be applied to the foliar surface of a plant or to the plant growth medium, such as soil or hydroponic solution, surrounding the plant. In silage operations, the microbial-based composition may be applied to a cut plant product during one or more stages in the silage process.

### CROSS-REFERENCE TO RELATED APPLICATIONS

The present application claims the benefit of U.S. Provisional Patent Application No. 62/611,080 filed Dec. 28, 2017, the disclosure of which, including any materials incorporated by reference therein, is hereby incorporated by reference it its entirety.

### TECHNICAL FIELD

The present disclosure relates to a microbial-based composition for use in, at least, plant growth promoting and silage applications, as well as related methods of use.

### BACKGROUND

Consortia of bacteria and other microorganisms can behave synergistically when combined in appropriate manners, such as by co-culturing them. Further, when combined with other agents, the new composition—the consortia and additional agents—can provide many useful applications. Those applications include, but are not limited to, plant growth promotion and silage applications. Relatedly, the composition is often dispersed in irrigation lines, as an agricultural spray, or combined with fertilizers.

Microbial-based compositions are desirable for use in many applications. First, the many thousands, if not millions, of potential microbial combinations between different types of microorganisms in consortiums presents a nearly unlimited amount of effective mixtures. Second, with respect to agricultural applications, microbes occur naturally in the soil and are hence a more natural choice to enhance a plant's growth. Third, and relatedly, many microbial-based compositions require no special disposal after their application.

The problem lies in with selecting the right microbial combination for a particular application, particularly with respect to a microbial-based composition for use in agricultural plant growth promotion applications and in silage applications. Many attempts to address one or the other of these applications have been made.

One attempt to address these needs can be seen with respect to the disclosure of WIPO International Publication No. 2013/029112, incorporated by reference in its entirety herein. Although this disclosure does provide for microbial compositions having complex consortia embodiment, and embodiments directed towards plant growth, this disclosure does not adequately provide for an adequately effective consortium having at least one lactic acid bacteria, yeast, fungus, and purple nonsulfur bacteria (PNSB), nor the specific benefits of the present disclosure.

Another attempt may be seen with respect to U.S. Patent Application Publication No. 2018/0235235, the disclosure of which is hereby incorporated in its entirety. While this disclosure does provide for targeted microbial consortiums directed to specific plant-growth applications, it fails to adequately provide for an embodiment directed towards silage operations. Additionally, it does not provide for the benefits associated with the specific microbial combinations of the present disclosure.

Still another attempt may be seen with respect to the disclosure of U.S. Patent Application Publication No. 2017/0245503, which is hereby incorporated by reference in its entirety. Although this disclosure does provide for microbial consortia directed to increasing a plant's root and shoot growth and larger overall biomass, it fails to adequately provide for a method of silage, does not provide for an adequately effective embodiment having at least one lactic acid bacteria, yeast, fungus, and PNSB, nor does this disclosure provide for the benefits associated with the specific microbial combinations of the present disclosure.

Accordingly, a need exists for an improved co-cultured microbial-based composition that provides for plant growth promotion and silage applications, as well as other applications and methods of use.

### SUMMARY

The present disclosure relates to a microbial-based composition having a co-cultured microorganism consortium in culture medium that, in some embodiments, includes one or more Acetobacter sp., Bacillus sp., Bifidobacterium sp., Enterococcus sp., Gluconacetobacter sp., Lactobacillus sp., Rhodopseudomonas sp., Saccharomyces sp., Pichia sp., and Trichoderma sp., as well as a carbon source and dechlorinated water.

Dechlorinated water, or chlorine-free water, may comprise water having 3 or fewer parts per million of elemental chlorine, any chloramine, sodium hypochlorite, calcium hypochlorite, or other chlorine-containing compound typically found in water. Preferably, the dechlorinated water of the microbial-based composition is free of detectable chlorine, chloramine, sodium hypochlorite, or calcium hypochlorite.

In at least one embodiment, the microbial-based composition provides qualities useful in the agricultural industry as a microbial inoculant that promotes plant health and plant growth. The microbial-based composition may be applied to the foliar surface of a plant or to the plant growth medium, such as soil or hydroponic solution, surrounding the plant. The present microbial-based composition may, when delivered to a plant's root, folair surface, or tissue system, improve nutrient availability and, in some embodiments, fertilizer efficiency. The presently disclosed microbial-based composition is believed to be stable under various conditions, to maintain high titers of microbes, possess an appreciable shelf life, and to be easily used.

In at least one embodiment, the microbial-based composition provides qualities useful in silage operations, wherein the microbial-based composition may be applied to a cut plant product during one or more stages of a silage process. At least one method may comprise applying the microbial-based composition to silage or during ensilage through an agricultural spray application such as but not limited to direct spray or as a tank mix in combination with other ingredients, which is then sprayed on the treated article. The amount of the composition sprayed, whether alone, together with an active agent, or with an active agent and other adjuvants or ingredients, varies depending on (i) the type of target agricultural article, and (ii) the desired effect, including the amount of penetration desired. In some applications, the microbial-based composition may be direct-sprayed, either alone or as an adjuvant, during ensilage or onto silage, to reduce average drying periods to 1 or 2 days while also simultaneously preventing microbial spoilage and augmenting ensilage processes at latter stages. This application may reduce damage to the cut grain due to putrefactive microorganisms and may increase the efficiency of the fermentation process.

In at least one embodiment, the microbial-based composition provides qualities useful as a bioadjuvant. An “adjuvant” is an agent that enhances the efficacy of one or more other agents. There are several types of adjuvants, such as inorganic compounds, mineral oils, bacterial products, non-bacterial organics, detergent-like delivering agents, and food-based oils. A “bioadjuvant” is a biologically-derived adjuvant that enhances the efficacy of another active ingredient or agent. Many bioadjuvant products are natural, biodegradable, and sometimes possess small molecular size. The microbial-based composition, in at least one embodiment, may be mixed with one or more ingredients and may enhance the effectiveness of one or more active ingredients.

One non-limiting example of the microbial-based composition as a bioadjuvant comprises adding the microbial-based composition to a tank mix containing fertilizer, pesticide, surfactant, chemical agent, or combination of these or other ingredients. In some embodiments, the microbial-based composition may modify or enhance the tank mix solution to improve dispersing, spreading, penetration, wetting, droplet size or other characteristics of the tank mix. In some embodiments, the resulting tank mix may be applied via agricultural spray, irrigation, or other method of application. fertilizers, soil amendments, non-ionic anionic and cationic surfactants, biofertilizers, or organic compounds such as urea, superphosphate, potash, or micronutrient fertilizers. When using the microbial-based composition in combination with other fertilizers, biofertilizers, organic compounds, or the like, the concentration of the fertilizer or other element can be reduced, or in some embodiments, the present microbial-based composition can serve as the sole fertilizer, biofertilizer, organic compound, or other such element. In either case, the soil microbial diversity of the inoculated soil may show improvement. Based on trial data, when the present disclosure is sprayed on one or more plants, at least one plant showed at least one positive benefit compared to unsprayed plants.

In at least one embodiment, the microbial-based composition is useful as a biosurfactant. Microbial-based composition-based surfactants, or “biosurfactants,” possess many advantages over other types of surfactants. By way of illustration, unlike many anionic surfactants, most biosurfactant products do not foam. This property is often an advantage in sprayers that have an agitator, or in any system where foam could disrupt water flow or pump suction. Also, the small molecular size of microbial-based composition-based biosurfactant products enables more effective penetration into the interfibrillar compartments of an article's structure. One example of microbial-based biosurfactants are the alkyl polyglucosides, a class of widely-used non-ionic surfactants that are derived from plant sugars. These compounds are biodegradable, environmentally friendly, possess good wetting properties, and present a low potential for phytotoxicity.

Within agriculture, a non-limiting example of the microbial-based composition useful as a biosurfactant may comprise a mixture of a water-soluble microbial-based composition with a water-based biostimulant, the combination of which is then applied to a plant's foliar portion(s), wherein the microbial-based composition acts as a biosurfactant by lowering the plant leaf s surface tension and increasing the biostimulant's penetration through the leafs waxy exterior and into its tissues.

Accordingly, some embodiments of the microbial-based composition may be used to improve and manage the microbial ecology of one or more crops, the condition of one or more plots of land, the efficiency of one or more irrigation systems, to implement one or more crop rotation schemes, or other application to improve soil quality for farming, reduce or eliminate unwanted pests, reduce chemicals used in various processes, and enhance fertilizer efficiency in agricultural applications.

In some embodiments, the microbial-based composition contains a microorganism consortium in culture medium. In one embodiment the microbial-based composition contains at least three microorganism strains. At least one microorganism may comprise a sulfide-utilizing microorganism, one or more species of lactic acid bateria, one or more Bacillus species, probiotic and phototrophic microorganisms, other types of bacteria, yeast, fungi, and mold species, as well as mixtures thereof. In some embodiments, the composition is free of pathogenic or putrefactive microorganisms and free of total coliform.

The microorganism consortium in culture medium may include a total number of microorganisms of about 1 to about 1 million colony forming units (CFU) per milliliter. In some embodiments, the composition includes a total number of microorganisms of about 100,000 to about 800,000 CFU per milliliter. In some embodiments, the composition includes a total number of microorganisms of about 250,000 to about 600,000 CFU per milliliter. In some embodiments, the composition includes a total number of microorganisms of at least 100,000 CFU per milliliter, at least 200,000 CFU per milliliter, at least 300,000 CFU per milliliter, at least 400,000 CFU per milliliter, at least 500,000 CFU per milliliter, at least 600,000 CFU per milliliter, at least 700,000 CFU per milliliter, at least 800,000 CFU per milliliter, at least 900,000 CFU per milliliter, at least 1,000,000 CFU per milliliter, at least 10,000,000 CFU per milliliter, at least 100,000,000 CFU per milliliter, at least 1,000,000,000 CFU per milliliter, at least 10,000,000,000 CFU per milliliter, at least 100,000,000,000 CFU per milliliter, or greater than at least 1,000,000,000,000 CFU per milliliter.

The microorganism consortium in culture medium of the present microbial-based composition may include living or non-living microorganisms. Compositions containing non-living microorganisms may contain extracts of the microorganisms. The extracts of microorganisms may include, by way of example, organic acid such as acetic acid, lactic acid, fatty acids, small chain peptides, and other compounds such as vitamins or polyols that are produced by microorganisms and that can elicit an effect on an environment regardless of the living status of the microorganism.

The microorganism consortium in culture medium may contain lactic acid bacteria (LAB) counts between 1.0E+1 to 1.0E+6 CFU per millileter. In some embodiments, the microorganism consortium in culture medium includes LAB counts of between 1.0E+5 to 8.0E+5 CFU per milliliter, 2.50E+5 to 6.0E+5 CFU per milliliter, about 3.0E+5 CFU per milliliter, or or up to or surpassing 1.0E+12 CFU per milliliter. The microorganism consortium in culture medium of the present microbial-based composition may comprise total lactic acid bacteria (LAB) counts between 1.0E+4 to 1.0E+6 cfu/mL, a pH value of <4.0, an HLB range of 13 to 16, E 24 values between 40 and 65, and percent titratable acidity between 0.6-3.2 percent, expressed as percent lactic acid.

In some embodiments, the microorganism consortium in culture medium may contain purple nonsulfur bacteria counts from about 1.0E+1 to about 1.0E+5, about 1.0E+1 to about 1.0E+4, between 1.0E+1 to 1.0E+6, between 1.0E+5 to 8.0E+5 CFU per milliliter, between 2.50E+5 to 6.0E+5 CFU per milliliter, between about 3.0E+5 CFU per milliliter, or up to or surpassing 1.0E+12 CFU per milliliter.

Moreover, the present disclosure relates to a bio-degradable, probiotics-based composition and method of use. The present disclosure relates to a microorganism consortia composition which includes lactic acid, sulfide-utilizing, probiotic and phototrophic microorganisms co-cultured to produce a composition useful in the agricultural industry as a bio-degradable, probiotics-based adjuvant composition, as a bioprotectant, and/or as an additive to pesticides for foliar sprays used on plants to control plant pathogens and insects. The composition may also be combined and applied, as an adjuvant, with chemical fertilizers to the soil to reduce the use of the chemical fertilizers, which improves the soil quality by increasing nutrient availability. The composition may also be used alone as a plant biostimulant to enhance root development in plants. The composition, when applied as an additive, enhances the quality of silage.

### BRIEF DESCRIPTION OF THE DRAWINGS

FIG. 1 is a photograph that illustrates the impact of at least one embodiment of the microbial-based composition as applied to fresh cut grain forage.

FIG. 2 shows two line charts. The first illustrates the impact of Tr1, Tr2, Tr3, and Control on rhizobia presence over time. The second illustrates the impact of Tr1, Tr2, Tr3, and Control on total biomass over time.

FIG. 3 illustrates a first MALDI-TOF profile of probiotics adjuvant composition as analyzed by Voyager DE STR MALDI-TOF MS system.

FIG. 4 illustrates a second MALDI-TOF profile of probiotics adjuvant composition as analyzed by Voyager DE STR MALDI-TOF MS system.

FIG. 5 is a line graph that shows stem height over time of Control, 1% microbial-based composition, 2% microbial-based composition, and 10% microbial-based composition as solid line with circle, dotted line with triangle, dashed line with triangle, and long dash with triangle, respectively.

FIG. 6 is a line graph that shows leaf number over time of Control, 1% microbial-based composition, 2% microbial-based composition, and 10% microbial-based composition as solid line with circle, dotted line with triangle, dashed line with triangle, and long dash with triangle, respectively.

FIG. 7 is a line graph that shows leaf area over time of Control, 1% microbial-based composition, 2% microbial-based composition, and 10% microbial-based composition as solid line with circle, dotted line with triangle, dashed line with triangle, and long dash with triangle, respectively.

FIG. 8A is a bar graph that shows number of mature leaves at experiment conclusion of Control, 1% microbial-based composition, 2% microbial-based composition, and 10% microbial-based composition left to right, respectively.

FIG. 8B is a bar graph that shows mature leaf area at experiment conclusion of Control, 1% microbial-based composition, 2% microbial-based composition, and 10% microbial-based composition left to right, respectively.

FIG. 9A is a bar graph that shows seedling plant height of Control, 1% microbial-based composition, 2% microbial-based composition, and 10% microbial-based composition left to right, respectively.

FIG. 9B is a bar graph that shows seedling root length of Control, 1% microbial-based composition, 2% microbial-based composition, and 10% microbial-based composition left to right, respectively.

FIG. 10A is a bar graph that shows transplant stem height at first transplant of Control, 1% microbial-based composition, 2% microbial-based composition, and 10% microbial-based composition left to right, respectively.

FIG. 10B is a bar graph that shows transplant root length at first transplant of Control, 1% microbial-based composition, 2% microbial-based composition, and 10% microbial-based composition left to right, respectively.

FIG. 11A is a bar graph that shows shoot germination rate at experiment conclusion of Control, 1% microbial-based composition, 2% microbial-based composition, and 10% microbial-based composition left to right, respectively.

FIG. 11B is a table showing overall germination rate of Control, 1% microbial-based composition, 2% microbial-based composition.

FIG. 11C is a table showing germination rate over time of Control, 1% microbial-based composition, 2% microbial-based composition.

### OBJECT OF THE DISCLOSURE

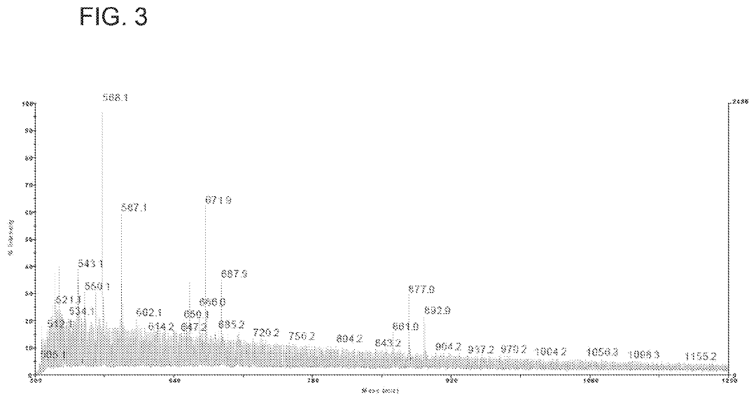

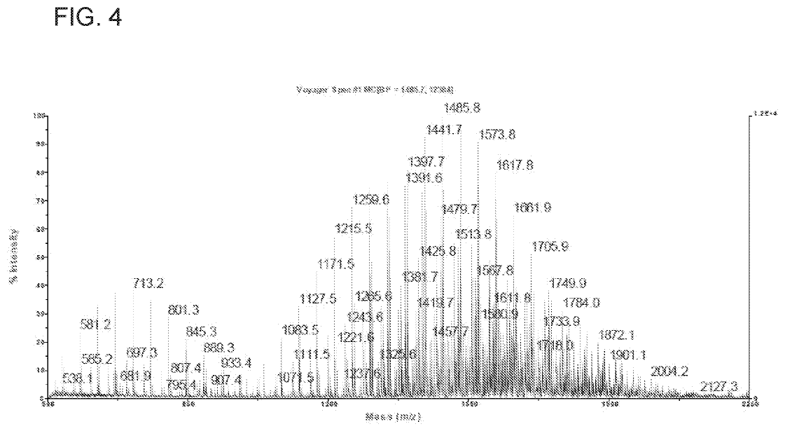

The present disclosure refers to a probiotics-based composition; characterized by a fermentation broth including but not limited to organic acids such as acetic acid, lactic acid, and other compounds such as vitamins, enzymes and small chain oligosaccharides with a MALDI-TOF mass spectra profile as shown in FIG. 3; along with total lactic acid bacteria (LAB) counts between 1.0E+4 to 1.0E+6 cfu/mL; wherein the final product has a pH value of <4.0, and percent titratable acidity expressed as Lactic Acid between 0.6-3.2, where the microorganism consortium comprises probiotic microbes containing at least 3 microorganisms.

In one embodiment, the microbial-based composition further comprises one or more of wetting, disbursing and/or solubilizing agents mixed in a suitable proportion with a deodorizer extracted from essential oils and additives required to keep the entire formulation stable over a period of at least one year. In this embodiment, the fermentation broth is further characterized by the presence of fatty acids, small chain peptides, biopolymers, and polyols, and has an HLB range of 13 to 16 and E 24 values between 40 and 65.

At least one microorganism present in the microbial-based composition is a sulfide-utilizing microorganism. Additional microorganisms that may be present include: lactic acid, probiotic and phototrophic microorganisms, as well as mixtures thereof. Microorganisms may be bacteria, yeast, fungi, mold species or mixtures thereof.

In another embodiment, the microbial-based composition includes at least one species of microorganism from lactic acid, probiotic, phototrophic, and sulfide-utilizing microorganism species. In an additional embodiment, the microbial-based composition may also include a purple non-sulfur bacteria species as the sulfide-utilizing microorganism.

The microbial-based composition includes a total number of microorganisms of about 1 to about 1 million colony forming units (CFU) per milliliter. Preferably, the microbial-based composition includes a total number of microorganisms of about 100,000 to about 800,000 CFU per milliliter. More preferably, the composition includes a total number of microorganisms of about 250,000 to about 600,000 CFU per milliliter. Most preferably, the microbial-based composition includes a total number of microorganisms of about 300,000 CFU per milliliter.

The microbial-based composition includes living or non-living microorganisms or fragments thereof. Compositions containing non-living microorganisms may contain extracts of the microorganisms. Such extracts may be considered a liquid fermentation product of the living microorganisms. The extracts of microorganisms include, by way of example, organic acid such as acetic acid, lactic acid, fatty acids, small chain peptides, and other compounds such as vitamins and/or polyols that are produced by microorganisms and can elicit an effect on an environment regardless of the living status of the microorganism.

The microbial-based composition may be mixed in suitable proportions with one or more of wetting, dispersing and/or solubilizing agents, along with a deodorizer extracted from essential oils and additives as needed to keep the entire formulation stable over a period of one year or more; this is characterized by a fermentation broth including but not limited to organic acids such as: acetic acid, lactic acid, fatty acids, small chain peptides, biopolymers and other compounds such as vitamins, enzymes, small chain oligosaccharides and polyols with a defined fingerprinting profile from metagenomics and metabolomics analysis; along with total lactic acid bacteria counts between 1.0E+4 to 1.0E+6 cfu/mL.

In one embodiment, purple sulphur bacteria is present and free of pathogenic or putrefactive microorganisms obtained by fermentation of selected probiotic strains in a selected medium under defined fermentation conditions.

The microbial-based composition contains Lactic Acid Bacteria counts between 1.0E+1 to 1.0E+6. Preferably, the microbial-based composition includes Lactic Acid Bacteria counts of about 1.0E+5 to about 8.0E+5 CFU per milliliter. More preferably, the microbial-based composition includes Lactic Acid Bacteria counts of about 2.50E+5 to about 6.0E+5 CFU per milliliter. Most preferably, the microbial-based composition includes a total number of microorganisms of about 3.0E+5 CFU per milliliter.

In one embodiment, the microbial-based composition is essentially free of purple sulfur bacteria. In another preferred embodiment, the microbial-based composition contains purple sulfur bacteria. In one embodiment, the composition contains purple sulfur bacteria at a level of from about 1.0E+1 to about 1.0E+5, and preferably, from about between 1.0E+1 to 1.0E+4.

The microbial-based composition is essentially free of pathogenic or putrefactive microorganisms. Preferably, the composition is essentially free of pathogenic mold and total coliform. Most preferably, pathogenic mold and total coliform are not detected.

The microbial-based composition relating to the present disclosure is completely free of phosphates, is non-toxic, and fully bio-degradable and is thus a green technology for the environment.

When using the inventive composition as an adjuvant, it may be used as an agricultural spray. One such use is in ensilage operations, where damage to the cut grain due to putrefactive microorganisms is greatly reduced. Simultaneously, the fermentation process is accomplished more efficiently.

When using the microbial-based composition in combination with other adjuvant agents and/or pesticides, the concentration of the compounds can be substantially reduced and/or replaced.

When using the microbial-based composition in combination with other fertilizers, the concentration of fertilizer can be reduced. Inoculated soil, at the same time, shows improvement of soil microbial diversity.

Most adjuvant agents in the prior art are petroleum-based and void of live cells. Therefore, the inventive composition offers a unique synergistic effect possibly via microbial ecology balancing and its novel biochemical metabolites.

The present disclosure refers to a probiotics-based adjuvant composition characterized by a fermentation broth including, but not limited to, organic acids such as acetic acid, lactic acid, and other compounds such as vitamins, enzymes and small chain oligosaccharides with a MALDITOF mass spectra profile as shown in FIG. 3; along with total lactic acid bacteria (LAB) counts between 1.0E+4 to 1.0E+6 cfu/mL; wherein the final product has a pH value of <4.0, and percent titratable acidity expressed as Lactic Acid between 0.6-3.2, where the microorganism consortium comprises probiotic microbes containing at least 3 microorganisms.

In another embodiment, the composition further comprises one or more of: wetting, dispersing, and/or solubilizing agents mixed in suitable proportions with a deodorizer extracted from essential oils and additives required to keep the entire formulation stable over a period of one year or more; the fermentation broth is further characterized by the presence of fatty acids, small chain peptides, biopolymers, and polyols, and has an HLB range of 13 to 16 and E 24 values between 40 and 65.

The probiotic organisms include a starting culture developed from a consortium of at least three co-cultured microorganisms, wherein at least one of those microorganisms is a sulphide-utilizing microorganism and at least two microorganisms are selected from the group consisting of lactic acid bacteria, probiotic microorganisms, and phototrophic microorganisms and a carbon source selected from the group consisting of rum, molasses, and the combination thereof.

The sulfide-utilizing microorganism(s) may be selected from the group comprising purple nonsulfur bacteria, chromatianeae, green sulfur bacteria, colorless sulfur bacteria, filamentous green bacteria, and any combination thereof. The lactic acid bacteria may be selected from the group comprising Lactobacillus, Lactococcus, Streptococcus, Enterococcus, Pediococcuss, Leuconostoc, and combinations thereof. The probiotic microorganism(s) may be selected from the group comprising Lactobacillus, Enterococcus, Bifidiobacterium, Bacillus, Pseudomonas, Sporolactobacillus, Micromonospora, Micrococcus, Rhodococcus, E. coli, and combinations thereof. The phototrophic microorganism(s) may be selected from the group comprising Rhodopseudomonas, Rhodob actor, Rhodopila, and combinations thereof.

More specifically, of the at least two microorganisms selected from the group consisting of lactic acid bacteria, probiotic microorganisms, and phototrophic microorganisms, they may be selected from the group consisting of Bacillus subtilis, Bifidobacterium animalis, Bifidobacterium bifidum, Bifidobacterium longum, Enterococcus lactis, Enterococcus thermophilus, Lactobacillus acidophilus, Lactobacillus bulgaricus, Lactobacillus casei, Lactobacillus fermentum, Lactobacillus plantarum, Rhodopseudomonas palustris, Rhodopseudomonas sphaeroides, Saccharomyces cerevisiae, and combinations thereof.

The starting culture may further comprise water, molybdenum, acetic acid, and/or alcohol.

### DETAILED DESCRIPTION

The present disclosure comprises a microbial-based composition having a co-cultured microorganism consortium in culture medium that, in some embodiments, includes one or more Acetobacter sp., Bacillus sp., Bifidobacterium sp., Enterococcus sp., Gluconacetobacter sp., Lactobacillus sp., Rhodopseudomonas sp., Saccharomyces sp., Pichia sp., and Trichoderma sp., as well as a carbon source and dechlorinated water. In some embodiments, the consortium includes one or more of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma vixens, together with at least one carbon source, such as molasses, and dechlorinated water.

In some embodiments, the microbial-based composition is useful in the agricultural industry as a plant growth promoting and silage-enhancing agent. For plant growth promotion applications, the microbial-based composition may be applied to the foliar surface of a plant or to the plant growth medium, such as soil or hydroponic solution, surrounding the plant. In silage operations, the microbial-based composition may be applied to a cut plant product during one or more stages in the silage process.

In other embodiments, the microbial-based composition may further comprise at least one additive. In some embodiments, the at least one additive may comprise a biosurfactant. In some embodiments, the at least one additive may comprise a fertilizer. In some embodiments, the at least one additive may comprise a microbial-based composition. In some embodiments, two or more additives may be provided. In such cases, the two or more additives may comprise a biosurfactant and a microbial-based composition. In some embodiments, the culture medium may comprise a nutrient broth (or “fermentation broth”).

### I. Composition

The microorganism consortium may comprise two or more co-cultured microorganisms that may, in some embodiments, comprise at least five co-cultured microorganisms. In some embodiments, at least one of the at least five co-cultured microorganisms is a sulfide-utilizing microorganism and at least two microorganisms are selected from the group consisting of lactic acid bacteria, probiotic microorganisms, and phototrophic microorganisms.

In some embodiments, the sulfide-utilizing microorganism(s) may comprise one or more species of purple nonsulfur bacteria or one or more species of purple sulfur bacteria, and may be selected from the group comprising purple non-sulfur bacteria, chromatianeae, green sulfur bacteria, colorless sulfur bacteria, filamentous green bacteria, and any combination thereof. Also, in some embodiments, the lactic acid bacteria may be selected from the group comprising Lactobacillus, Lactococcus, Streptococcus, Enterococcus, Pediococcuss, Leuconostoc, and combinations thereof. As well, the probiotic microorganism(s) may be selected from the group comprising Lactobacillus, Enterococcus, Bifidiobacterium, Bacillus, Pseudomonas, Sporolactobacillus, Micromonospora, Micrococcus, Rhodococcus, E. coli, and combinations thereof. Moreover, in some embodiments, the phototrophic microorganism(s) may be selected from the group comprising Rhodopseudomonas, Rhodobactor, Rhodopila, and combinations thereof.

More specifically, the at least two microorganisms may be selected from the group comprising Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, Trichoderma virens, and combinations thereof.

Any microorganism of the microbial-based composition, whether in nutrient broth or in isolation, may be provided as liquid fermentation products. By way of illustration and not limitation, the microorganism consortium may comprise liquid Bacillus subtilis, liquid Bifidobacterium animalis, liquid Bifidobacterium bifidum product, liquid Bifidobacterium longum product, liquid Enterococcus lactis product, liquid Enterococcus thermophilus product, liquid Lactobacillus acidophilus product, liquid Lactobacillus bulgaricus product, liquid Lactobacillus casei product, liquid Lactobacillus fermentum product, liquid Lactobacillus plantarum product, liquid Rhodopseudomonas palustris product, liquid Rhodopseudomonas sphaeroides product, liquid Saccharomyces cerevisiae product, and combinations thereof.

In some embodiments, the microbial-based composition may comprise at least one carbon source, dechlorinated water, molybdenum, acetic acid, an alcohol, or a combination thereof.

The microbial-based composition may, in some embodiments, also comprise other elements, such as for example peptides, casein peptones, vitamins (such as vitamins A, D, E, and K, vitamin C, thiamin, riboflavin, niacin, pantothenic acid, biotin, vitamin B6 (pyridoxine), folate or folic acid, and vitamin B12 (cyanocobalamin)), elements of lysogeny broth, calcium, phosphorus, potassium, sodium, chlorine, sulfur, copper, iron, fluoride, vanadium, nitrogen, sulfur, magnesium, chromium, iodine, selenium, zinc, tryptone and other amino acids, yeast extract, distillers dried grains with solubles, fish fertilizer, and any other element known to not impede the growth of microorganisms.

In some embodiments, the microbial-based composition may additionally comprise at least one enzyme, such as but not limited to oxidoreductases, transferases, hydrolases, lyases, isomerases, ligases, lipases, proteases, peptidases, lipases, phosphatases, esterases, amylases, nucleases, carboxylases, carbohydrases, mutases, epimerases, phytases and any other enzyme or class of enzymes. In some embodiments, one or more microorganism may produce one or more enzymes in the culture medium. In some embodiments, one or more enzymes may be isolated separately from the culture medium and added to the culture medium.

The microbial-based composition may also comprise, in some embodiments, one or more organic acids, such as acetic acid, carboxylic acid, lactic acid, formic acid, propionic acid, butanoic acid, isobutyric acid, 3-methyl butanoic acid, methyl acetate ethyl acetate, propyl acetate, butyl acetate, isobutyl acetate, and 2-methyl butyl acetate. In one embodiment, the acetic acid is included by using vinegar, glacial acetic acid or a dilution-in-water thereof. The weight fraction of the acetic acid or other organic acid in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The microbial-based composition may also comprise one or more essential oils (or “natural oils”) that may, in some embodiments, serve as a deodorant, a deordorizer, an insect repellant, an insecticide, an adjuvant that enhances one or more of the composition's other properties, or as a fertilizer or biostimulant. Such essential oils or natural oils may include, without limitation, African lemon bush (Lippia javanica) oil, anise oil, bay oil, bergamot oil, boronia oil, canola oil, carrot oil, cassia oil, catnip oil, cedarwood oil, chamomile oil, cinnamon oil, citronella oil, clary sage oil, clove oil, cypress oil, eucalyptus oil, galbanum oil, garlic oil, ginger oil, geranium oil, grapefruit oil, hazelnut oil, jasmine oil, jojoba oil, lavender oil, lavandin oil, lemon oil, lime oil, mandarin oil, nutmeg oil, orange oil, palma rosa oil, patchouli oil, Peru balsams, peppermint oil, rosemary oil, rosewood oil, sage oil, sandalwood oil, spear mint oil, star anise oil, tea tree oil, tangerine oil, thyme oil, tolu, verbena oil, white clover oil, ylang ylang oil, and combinations thereof.

The microbial-based composition may further comprise one or more carbon sources, including rum, molasses, glucose, starch, cellulose, fructose, sucrose, or the like. In one embodiment, the carbon source is plant material such as silage. In another embodiment, the carbon source is a plant material from the grass family Poaceae. In some embodiments, the carbon source may comprise a relatively unrefined plant material, such as silage, stover, chaff, grass, stalks, leaves, and the like. In other embodiments, the carbon source comprises a more refined plant material, such as flour, syrup, molasses, or the like. In another embodiment, the carbon source comprises purified or semi-purified organic molecules, such as protein, fat, fatty acids, carbohydrates, or the like. In a more specific embodiment, the carbon source comprises a bran from grain, such as, e.g., rice bran, or a syrup or molasses from sugar cane.

Further examples of suitable carbon sources include, without limitation, peptone, yeast extract, amino acids, other sugars such as arabinose, mannose, glucosamine, maltose, sugar cane, molasses, rum, and the like; salts of organic acids such as acetic acid, fumaric acid, adipic acid, propionic acid, citric acid, gluconic acid, malic acid, pyruvic acid, malonic acid and the like; alcohols such as ethanol, glycerol, and the like; or oil or fat such as soybean oil, rice bran oil, olive oil, corn oil, and sesame oil. The amount of the carbon source added varies according to the kind of carbon source and is typically between 1 to 100 grams per liter of medium. The weight fraction of the carbon source in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition. Preferably, molasses is contained in the medium as a carbon source, at a concentration of about 2 to 20% (w/v). More preferably, the molasses is at a concentration of about 8 to 12% (w/v).

The microbial-based composition may further comprise dechlorinated water. In some embodiments, the chlorine-free water (or “dechlorinated” water) may be obtained by exposing the water to air and allowing the chlorine to evaporate, by carbon-filtering the water, or any by other known means of dechlorinating water. In some embodiments, however, the water may comprise filtered water, twice filtered water, treated water, water having additives, filtered sewage, runoff water, greywater, tap water, well water, carbonated water, seawater, lakewater, pondwater, rainwater, any of which may be captured, redirected, modified, or produced by known means.

The microbial-based composition may also comprise at least one additive. In some embodiments, the at least one additive may comprise a fertilizer or a biofertilizer. In some embodiments, the at least one additive may comprise a first additive and a second additive, such as but not limited to a biofertilizer or a fertilizer.

In embodiments wherein Acetobacter ghanensis comprises one or more microbes of the microorganism consortium, illustrative strains may include, but are not limited to, Acetobacter ghanensis, including but not limited to Acetobacter ghanensis DSM-18895.

In embodiments wherein Acetobacter pasteurianus comprises one or more microbes of the microorganism consortium, illustrative strains may include, but are not limited to, Acetobacter pasteurianus subsp. pasteurianus (Hansen) Beijerinck and Folpmers ATCC 12879, Acetobacter pasteurianus subsp. pasteurianus (Hansen) Beijerinck and Folpmers ATCC 23757, Acetobacter pasteurianus subsp. pasteurianus (Hansen) Beijerinck and Folpmers ATCC 33443, Acetobacter pasteurianus subsp. pasteurianus (Hansen) Beijerinck and FolpmersATCC 33445, Acetobacter pasteurianus subsp. pasteurianus (Hansen) Beijerinck and Folpmers ATCC 23650, Acetobacter pasteurianus subsp. pasteurianus (Hansen) Beijerinck and Folpmers ATCC 23754, Acetobacter pasteurianus subsp. pasteurianus (Hansen) Beijerinck and Folpmers ATCC 12873, Acetobacter pasteurianus subsp. pasteurianus (Hansen) Beijerinck and Folpmers ATCC 7839, Acetobacter pasteurianus subsp. pasteurianus (Hansen) Beijerinck and Folpmers ATCC 23761, Acetobacter pasteurianus subsp. pasteurianus (Hansen) Beijerinck and Folpmers ATCC 23756, Acetobacter pasteurianus subsp. pasteurianus (Hansen) Beijerinck and Folpmers ATCC 9325, Acetobacter pasteurianus subsp. pasteurianus (Hansen) Beijerinck and Folpmers ATCC 9433, Acetobacter pasteurianus subsp. pasteurianus (Hansen) Beijerinck and Folpmers ATCC 23764, Acetobacter pasteurianus subsp. pasteurianus (Hansen) Beijerinck and Folpmers ATCC 23759, Acetobacter pasteurianus subsp. pasteurianus (Hansen) Beijerinck and Folpmers ATCC 6439, Acetobacter pasteurianus subsp. pasteurianus (Hansen) Beijerinck and Folpmers ATCC 23758, Acetobacter pasteurianus subsp. pasteurianus (Hansen) Beijerinck and Folpmers ATCC 23760, Acetobacter pasteurianus subsp. pasteurianus (Hansen) Beijerinck and Folpmers ATCC 12877, Acetobacter pasteurianus subsp. pasteurianus (Hansen) Beijerinck and Folpmers ATCC 23752, Acetobacter pasteurianus subsp. pasteurianus (Hansen) Beijerinck and Folpmers ATCC 838, Acetobacter pasteurianus subsp. pasteurianus (Hansen) Beijerinck and Folpmers ATCC 9323, Acetobacter pasteurianus subsp. pasteurianus (Hansen) Beijerinck and Folpmers ATCC 19877, Acetobacter pasteurianus subsp. pasteurianus (Hansen) Beijerinck and Folpmers ATCC 9432, Acetobacter Acetobacter Beijerinck) aceti (Pasteur) Beijerinck ATCC 23747, Acetobacter sp. ATCC 21761, Acetobacter pasteurianus subsp. pasteurianus (Hansen) Beijerinck and Folpmers ATCC 33444, Acetobacter pasteurianus subsp. pasteurianus (Hansen) Beijerinck and Folpmers ATCC 12879, Acetobacter pasteurianus subsp. pasteurianus (Hansen) Beijerinck and Folpmers ATCC 23757, Acetobacter pasteurianus subsp. pasteurianus (Hansen) Beijerinck and Folpmers ATCC 33443, Acetobacter pasteurianus subsp. pasteurianus (Hansen) Beijerinck and Folpmers ATCC 33445, Acetobacter pasteurianus subsp. pasteurianus (Hansen) Beijerinck and Folpmers ATCC 23650, Acetobacter pasteurianus subsp. pasteurianus (Hansen) Beijerinck and Folpmers ATCC 23754, Acetobacter pasteurianus subsp. pasteurianus (Hansen) Beijerinck and Folpmers ATCC 12873, Acetobacter pasteurianus subsp. pasteurianus (Hansen) Beijerinck and Folpmers ATCC 9322, Acetobacter pasteurianus subsp. pasteurianus (Hansen) Beijerinck and Folpmers ATCC 7839, Acetobacter pasteurianus subsp. pasteurianus (Hansen) Beijerinck and Folpmers ATCC 23761, Acetobacter pasteurianus subsp. pasteurianus (Hansen) Beijerinck and Folpmers ATCC 23756, Acetobacter pasteurianus subsp. pasteurianus (Hansen) Beijerinck and Folpmers ATCC 9325, Acetobacter pasteurianus subsp. pasteurianus (Hansen) Beijerinck and Folpmers ATCC 9433, Acetobacter pasteurianus subsp. pasteurianus (Hansen) Beijerinck and Folpmers ATCC 23764, Acetobacter pasteurianus subsp. pasteurianus (Hansen) Beijerinck and Folpmers ATCC 23759, Acetobacter pasteurianus subsp. pasteurianus (Hansen) Beijerinck and Folpmers ATCC 6439, Acetobacter pasteurianus subsp. pasteurianus (Hansen) Beijerinck and Folpmers ATCC 23758, Acetobacter pasteurianus subsp. pasteurianus (Hansen) Beijerinck and Folpmers ATCC 23760, Acetobacter pasteurianus subsp. pasteurianus (Hansen) Beijerinck and Folpmers ATCC 12877, Acetobacter pasteurianus subsp. pasteurianus (Hansen) Beijerinck and Folpmers ATCC 23752, Acetobacter pasteurianus subsp. pasteurianus (Hansen) Beijerinck and Folpmers ATCC 838, Acetobacter pasteurianus subsp. pasteurianus (Hansen) Beijerinck and Folpmers ATCC 9323, Acetobacter pasteurianus subsp. pasteurianus (Hansen) Beijerinck and Folpmers ATCC 19877, or Acetobacter pasteurianus subsp. pasteurianus (Hansen) Beijerinck and Folpmers ATCC 9432.

In embodiments wherein Bacillus subtilis comprises one or more microbial organisms of the microorganism consortium, illustrative strains may include, but are not limited to, Bacillus subtilis Ehrenberg Cohn ATCC 15477 Strain Designations: M-24-1, Bacillus subtilis Ehrenberg Cohn ATCC 31098, Bacillus subtilis Ehrenberg Cohn ATCC 21951, Bacillus subtilis Ehrenberg Cohn ATCC 21008, Bacillus subtilis 21007, Bacillus subtilis 21358, Bacillus subtilis 21005, Bacillus subtilis 21006, Bacillus subtilis 21356, Bacillus subtilis 19220, 53683, Bacillus subtilis 19219, B. subtilis 53685, Bacillus subtilis 21357, B. subtilis 21398, B. subtilis 53689, Bacillus subtilis Ehrenberg Cohn ATCC 21770, Bacillus subtilis natto, and Bacillus subtilis 19217.

In some embodiments wherein Bifidobacterium bifidum comprises one or more microbial organisms of the microorganism consortium, illustrative strains may include, but are not limited to, Bifidobacterium bifidum Tissier Orla-Jensen ATCC 29521, Bifidobacterium bifidum Tissier Orla-Jensen ATCC BAA-2850, Bifidobacterium bifidum Tissier Orla-Jensen ATCC 11863, Bifidobacterium bifidum Tissier Orla-Jensen ATCC 35914, and Bifidobacterium bifidum Tissier Orla-Jensen ATCC 15696.

As well, In some embodiments wherein Bifidobacterium longum comprises one or more microbial organisms of the microorganism consortium, illustrative strains may include, but are not limited to, Bifidobacterium longum subsp. infantis Reuter Mattarelli et al. ATCC 15697, Bifidobacterium longum subsp. longum Reuter ATCC 15707, Bifidobacterium longum subsp. suis Matteuzzi et al. Mattarelli et al. ATCC 27533, Bifidobacterium longum subsp. infantis Reuter Mattarelli et al. ATCC 25962, Bifidobacterium longum subsp. infantis Reuter Mattarelli et al. ATCC 15702, Bifidobacterium longum Reuter ATCC BAA-999, Bifidobacterium longum subsp. suis Matteuzzi et al. Mattarelli et al. ATCC 27532, Bifidobacterium longum Reuter ATCC 15708, Bifidobacterium longum Reuter ATCC 55815, Bifidobacterium longum Reuter ATCC 55813, Bifidobacterium longum Reuter ATCC 55818, Bifidobacterium longum Reuter ATCC 55817, Bifidobacterium longum subsp. suis (Matteuzzi et al.) Mattarelli et al. ATCC 27531, Bifidobacterium longum Reuter ATCC 51870, Bifidobacterium longum Reuter ATCC 35183, Bifidobacterium longum subsp. infantis Reuter Mattarelli et al. ATCC 15697D-5, Bifidobacterium longum subsp. infantis Reuter Mattarelli et al. ATCC 17930, Bifidobacterium longum subsp. longum Reuter ATCC BAA-2753, Bifidobacterium longum Reuter ATCC 55814, or Bifidobacterium longum Reuter ATCC 55816.

In some embodiments wherein Enterococcus lactis comprises one or more microbial organisms of the microorganism consortium, illustrative strains may include, but are not limited to, Enterococcus lactis BT159 T, Enterococcus lactis CCM 8412, Enterococcus lactis DSM 23655 Enterococcus lactis LMG 25958, Enterococcus lactis Morandi BT159, or any other species of Enterococcus lactis.

In some embodiments wherein Enterococcus thermophilus comprises one or more microbial organisms of the microorganism consortium, illustrative strains may include, but are not limited to, Streptococcus salivarius subsp. thermophilus (Orla-Jensen) Farrow and Collins (ATCC BAA-491D-5), Streptococcus salivarius subsp. thermophilus (Orla-Jensen) Farrow and Collins (ATCC 14485), Streptococcus salivarius subsp. thermophilus (Orla-Jensen) Farrow and Collins (ATCC 19258), Streptococcus salivarius subsp. thermophilus (Orla-Jensen) Farrow and Collins (ATCC BAA-491), Streptococcus salivarius subsp. thermophilus (Orla-Jensen) Farrow and Collins (ATCC BAA-250), Streptococcus salivarius subsp. thermophilus (Orla-Jensen) Farrow and Collins (ATCC BAA-250D-5), Enterococcus faecium (Orla-Jensen) Schleifer and Kilpper-Balz (ATCC 51836, deposited as Streptococcus thermophilus Orla-Jensen), Streptococcus thermophilus NCIMB 10387, Streptococcus salivarius subsp. thermophilus NCIMB 702393, Streptococcus thermophilus NCIMB 702557, Streptococcus salivarius subsp. thermophilus NCIMB 702641, or any other related strain of Enterococcus thermophilus.

In embodiments wherein Gluconacetobacter diazotrophicus comprises one or more microbes of the microorganism consortium, illustrative strains may include, but are not limited to, Gluconacetobacter diazotrophicus Gillis et al. Yamada et al. ATCC 49037, Gluconacetobacter diazotrophicus Gillis et al. Yamada et al. ATCC 49039, Gluconacetobacter diazotrophicus Gillis et al. Yamada et al. ATCC 49038, Gluconacetobacter diazotrophicus Gillis et al. Yamada et al. ATCC 49037D-5. Gluconacetobacter diazotrophicus (Gillis et al.) Yamada et al. ATCC 49037D-5, Gluconacetobacter diazotrophicus (Gillis et al.) Yamada et al. ATCC 49039, Gluconacetobacter xylinus (Brown) Yamada et al. ATCC 700178, Gluconacetobacter xylinus (Brown) Yamada et al. ATCC 23767, Gluconacetobacter diazotrophicus (Gillis et al.) Yamada et al. ATCC 49037D-5, and Gluconacetobacter diazotrophicus (Gillis et al.) Yamada et al. ATCC 49039.

In embodiments wherein Lactobacillus acetotolerans comprises one or more microbes of the microorganism consortium, illustrative strains may include, but are not limited to, Lactobacillus acetotolerans Entani et al. ATCC 43578, Lactobacillus acetotolerans Entani et al. ATCC 27742, Lactobacillus acidophilus (Moro) Hansen and Mocquot ATCC 43121, Lactobacillus rhamnosus (Hansen) Collins et al. ATCC 21052D-5, Lactobacillus rhamnosus (Hansen) Collins et al. ATCC 7469, Lactobacillus brevis (Orla-Jensen) Bergey et al. ATCC 14869, Lactobacillus acidophilus (Moro) Hansen and Mocquot ATCC BAA-2832, Lactobacillus acetotolerans Entani et al. ATCC 43578, and Lactobacillus acetotolerans Entani et al. ATCC 27742.

In some embodiments wherein Lactobacillus acidophilus comprises one or more microbial organisms of the microorganism consortium, illustrative strains may include, but are not limited to, Lactobacillus acidophilus Moro Hansen and Mocquot ATCC 4356, Lactobacillus acidophilus Moro Hansen and Mocquot ATCC 4355, Lactobacillus acidophilus Moro Hansen and Mocquot ATCC 4796, Lactobacillus acidophilus Moro Hansen and Mocquot ATCC 4357, Lactobacillus acidophilus Moro Hansen and Mocquot ATCC BAA-2832, Lactobacillus acidophilus Moro Hansen and Mocquot ATCC 53546, Lactobacillus acidophilus Moro Hansen and Mocquot ATCC 53544, Lactobacillus acidophilus Moro Hansen and Mocquot ATCC 11975, Lactobacillus acidophilus Moro Hansen and Mocquot ATCC 314, Lactobacillus acidophilus Moro Hansen and Mocquot ATCC 832, Lactobacillus acidophilus Moro Hansen and Mocquot ATCC 9224, Lactobacillus acidophilus Moro Hansen and Mocquot ATCC 43121, Lactobacillus acidophilus Moro Hansen and Mocquot ATCC 53671, Lactobacillus acidophilus: B6T7 PTA-4482, Lactobacillus acidophilus Moro Hansen and Mocquot ATCC BAA-2845, Lactobacillus acidophilus Moro Hansen and Mocquot ATCC 4357D-5, Lactobacillus animalis LA51 Deposited as Lactobacillus acidophilus PTA-6750, Lactobacillus acidophilus isolated from bovine feed LA45 PTA-6749, Lactobacillus acidophilus M35 PTA-6751, Lactobacillus acidophilus L411 PTA-6820, Lactobacillus gallinarum Fujisawa et al. ATCC 33199, Lactobacillus amylovorus Nakamura ATCC 33198, Lactobacillus gasseri Lauer and Kandler ATCC 19992, Lactobacillus johnsonii Fujisawa et al. ATCC 11506, Lactobacillus johnsonii Fujisawa et al. ATCC 33200, Lactobacillus johnsonii Fujisawa et al. ATCC 332, Lactobacillus rhamnosus Hansen Collins et al. ATCC 53103, Lactobacillus crispatus Brygoo and Aladame Moore and Holdeman ATCC 33197, Lactobacillus crispatus Brygoo and Aladame Moore and Holdeman ATCC 53545, Lactobacillus rhamnosus Hansen Collins et al. ATCC 21052, Bacillus sp. ATCC 31283 (Lactobacillus acidophilus Moro Hansen and Mocquot), Lactobacillus casei Orla-Jensen Hansen and Lessel ATCC 4646 (Lactobacillus acidophilus Moro Hansen and Mocquot), Lactobacillus gasseri Lauer and Kandler ATCC 9857 (Lactobacillus acidophilus Moro Hansen and Mocquot), Lactobacillus fermentum Beijerinck ATCC 11976 (Lactobacillus acidophilus Moro Hansen and Mocquot), Lactobacillus gasseri Lauer and Kandler ATCC 29601 (Lactobacillus acidophilus Moro Hansen and Mocquot), Lactobacillus paracasei subsp. paracasei Collins et al. ATCC 11974 (Lactobacillus acidophilus Moro Hansen and Mocquot), Lactobacillus johnsonii Fujisawa et al. ATCC 53672 (Lactobacillus acidophilus Moro Hansen and Mocquot), or Lactobacillus crispatus Brygoo and Aladame Moore and Holdeman ATCC 55221 (Lactobacillus acidophilus Moro Hansen and Mocquot).

In some embodiments wherein Lactobacillus bulgaricus comprises one or more microbial organisms of the microorganism consortium, illustrative strains may include, but are not limited to, Lactobacillus bulgaricus (Lactobacillus delbrueckii subsp. bulgaricus) selected from Lactobacillus delbrueckii subsp. bulgaricus Orla-Jensen Weiss et al. ATCC 11842 (Lactobacillus bulgaricus Orla-Jensen Rogosa and Jensen), Lactobacillus delbrueckii subsp. bulgaricus (Orla-Jensen) Weiss et al. (ATCC BAA-365D), Lactobacillus delbrueckii subsp. bulgaricus (Orla-Jensen) Weiss et al. (ATCC 11842D-5), Lactobacillus delbrueckii subsp. bulgaricus (Orla-Jensen) Weiss et al. (ATCC BAA-365), Lactobacillus delbrueckii subsp. bulgaricus (Orla-Jensen) Weiss et al. (ATCC 11842), Lactobacillus salivarius subsp. salivarius Rogosa et al. (ATCC 11741), Lactobacillus helveticus (Orla-Jensen) Bergey et al. (ATCC 7995D deposited as Lactobacillus delbrueckii sub sp. bulgaricus (Orla-Jensen) Weiss et al.), Lactobacillus leichmannii (Henneb erg) Bergey et al. (ATCC 21815 deposited as Lactobacillus bulgaricus (Orla-Jensen) Rogosa and Jensen), Lactobacillus helveticus (Orla-Jensen) Bergey et al. (ATCC 55163 deposited as Lactobacillus delbrueckii sub sp. bulgaricus (Orla-Jensen) Weiss et al.), Lactobacillus delbrueckii subsp. lactis NCIMB 7854, Lactobacillus delbrueckii subsp. lactis NCIMB 8117, Lactobacillus delbrueckii sub sp. lactis NCIMB 8118, Lactobacillus delbrueckii sub sp. delbrueckii NCIMB 8130, Lactobacillus delbrueckii sub sp. delbrueckii NCIMB 8183, Lactobacillus delbrueckii sub sp. lactis NCIMB 8183, Lactobacillus delbrueckii subsp. bulgaricus NCIMB 11778, or Lactobacillus delbrueckii subsp. bulgaricus NCIMB 701438.

In some embodiments wherein Lactobacillus casei comprises one or more microbial organisms of the microorganism consortium, illustrative strains may include, but are not limited to, Lactobacillus casei DSM 28872, Lactobacillus casei PCM B/00080, Lactobacillus casei DSM 28872, Lactobacillus casei (Orla-Jensen) Hansen and Lessel (ATCC 39539), Lactobacillus casei (Orla-Jensen) Hansen and Lessel (ATCC 39392), Lactobacillus casei (Orla-Jensen) Hansen and Lessel (ATCC 4940), Lactobacillus casei (Orla-Jensen) Hansen and Lessel (ATCC 393), Lactobacillus casei (Orla-Jensen) Hansen and Lessel (ATCC 334), Lactobacillus casei (Orla-Jensen) Hansen and Lessel (ATCC 27139), Lactobacillus casei (Orla-Jensen) Hansen and Lessel (ATCC BAA-2843), Lactobacillus casei (Orla-Jensen) Hansen and Lessel (ATCC 4646), Lactobacillus rhamnosus (Hansen) Collins et al. (ATCC 7469), Lactobacillus casei (Orla-Jensen) Hansen and Lessel (ATCC 4007), Lactobacillus paracasei subsp. paracasei Collins et al. (ATCC 11578), Lactobacillus rhamnosus (Hansen) Collins et al. (ATCC 14957 deposited as Lactobacillus casei (Orla-Jensen) Hansen and Lessel), Lactobacillus sp. (ATCC 14435 deposited as Lactobacillus casei (Orla-Jensen) Hansen and Lessel), Lactobacillus rhamnosus (Hansen) Collins et al. (ATCC 13075 deposited as Lactobacillus casei subsp. rhamnosus Hansen), Lactobacillus rhamnosus (Hansen) Collins et al. (ATCC 12116 deposited as Lactobacillus casei (Orla-Jensen) Hansen and Lessel), Lactobacillus rhamnosus (Hansen) Collins et al. (ATCC 11981 deposited as Lactobacillus casei subsp. rhamnosus Hansen), Lactobacillus paracasei subsp. paracasei Collins et al. (ATCC 11582 deposited as Lactobacillus casei (Orla-Jensen) Hansen and Lessel), Lactobacillus rhamnosus (Hansen) Collins et al. (ATCC 9595 deposited as Lactobacillus casei (Orla-Jensen) Hansen and Lessel), Lactobacillus rhamnosus (Hansen) Collins et al. (ATCC 8530 deposited as Lactobacillus casei subsp. rhamnosus Hansen), Lactobacillus rhamnosus (Hansen) Collins et al. (ATCC 7469a deposited as Lactobacillus casei (Orla-Jensen) Hansen and Lessel), Lactobacillus paracasei subsp. paracasei Collins et al. (ATCC 29599 deposited as Lactobacillus casei (Orla-Jensen) Hansen and Lessel, Lactobacillus rhamnosus (Hansen) Collins et al. (ATCC 27773 deposited as Lactobacillus casei (Orla-Jensen) Hansen and Lessel, Lactobacillus paracasei subsp. tolerans Collins et al. (ATCC 25599 deposited as Lactobacillus casei subsp. tolerans Abo-Elnaga and Kandler), Lactobacillus paracasei subsp. paracasei Collins et al. (ATCC 25598 deposited as Lactobacillus casei subsp. pseudoplantarum Abo-Elnaga and Kandler), Lactobacillus paracasei subsp. paracasei Collins et al. (ATCC 25303 deposited as Lactobacillus casei (Orla-Jensen) Hansen and Lessel), Lactobacillus paracasei subsp. paracasei Collins et al. (ATCC 25302, deposited as Lactobacillus casei (Orla-Jensen) Hansen and Lessel), Actinomyces naeslundii Thompson and Lovestedt (ATCC 19039 deposited as Actinomyces naeslundii Thompson and Lovestedt), Lactobacillus casei (Orla-Jensen) Hansen and Lessel (ATCC 4913 deposited as Lactobacillus delbrueckii (Leichmann) Beijerinck), Lactobacillus casei (Orla-Jensen) Hansen and Lessel (ATCC 15008 deposited as Lactobacillus casei subsp. casei (Orla-Jensen) Hansen and Lessel), Lactobacillus rhamnosus (Hansen) Collins et al. (ATCC 39595) deposited as Lactobacillus casei subsp. rhamnosus Hansen), Lactobacillus sp. (ATCC 49178 deposited as Lactobacillus casei (Orla-Jensen) Hansen and Lessel), Lactobacillus paracasei subsp. paracasei Collins et al. (ATCC 25180, deposited as Lactobacillus casei subsp. alacotosus Mills and Lessel), Lactobacillus rhamnosus (Hansen) Collins et al. (ATCC 11982 deposited as Lactobacillus casei subsp. rhamnosus Hansen), Lactobacillus paracasei subsp. paracasei Collins et al. (ATCC 27216 deposited as Lactobacillus casei subsp. alacotosus Mills and Lessel), Lactobacillus paracasei subsp. paracasei Collins et al. (ATCC 335 deposited as Lactobacillus casei (Orla-Jensen) Hansen and Lessel), or Lactobacillus paracasei subsp. paracasei Collins et al. (ATCC 27092 deposited as Lactobacillus casei (Orla-Jensen) Hansen and Lessel).

In some embodiments wherein Lactobacillus fermentum comprises one or more microbial organisms of the microorganism consortium, illustrative strains may include, but are not limited to, Lactobacillus fermentum Beijerinck ATCC 14932, Lactobacillus fermentum Beijerinck ATCC 14931, Lactobacillus fermentum Beijerinck ATCC 11739, Lactobacillus fermentum Beijerinck ATCC 9338, Lactobacillus fermentum Beijerinck ATCC 11976, Lactobacillus fermentum Beijerinck ATCC 23271, Lactobacillus fermentum Beijerinck ATCC 11740, Lactobacillus fermentum Beijerinck ATCC 8289, Lactobacillus fermentum B-54 55884, Lactobacillus reuteri Kandler et al. ATCC 23272 (Lactobacillus fermentum Beijerinck), Lactobacillus fermentum Beijerinck ATCC BAA-2842, Lactobacillus fermentum NCIMB 2797, Lactobacillus fermentum NCIMB 5220, Lactobacillus fermentum NCIMB 5221, Lactobacillus fermentum NCIMB 6991, Lactobacillus fermentum NCIMB 7230, Lactobacillus fermentum NCIMB 8028, Lactobacillus fermentum NCIMB 8828, Lactobacillus fermentum NCIMB 8829, Lactobacillus fermentum NCIMB 8830, Lactobacillus fermentum NCIMB 8961, Lactobacillus fermentum NCIMB 8962, Lactobacillus fermentum NCIMB 11840, Lactobacillus fermentum NCIMB 12116, Lactobacillus fermentum NCIMB 12117, Lactobacillus fermentum NCIMB 12118, Lactobacillus fermentum NCIMB 12119, Lactobacillus fermentum NCIMB 700335, Lactobacillus fermentum NCIMB 700479, Lactobacillus fermentum NCIMB 700927, Lactobacillus fermentum NCIMB 700928, Lactobacillus fermentum NCIMB 701052, Lactobacillus fermentum NCIMB 701068, Lactobacillus fermentum NCIMB 701751, Lactobacillus fermentum NCIMB 702341, or Lactobacillus fermentum NCIMB 702342.

In some embodiments wherein Lactobacillus parafarraginis comprises one or more microbial organisms of the microorganism consortium, illustrative strains may include, but are not limited to, Lactobacillus parafarraginis NCIMB 15108 or Lactobacillus parafarraginis NCIMB 702943.

In some embodiments wherein Lactobacillus plantarum comprises one or more microbial organisms of the microorganism consortium, illustrative strains may include, but are not limited to, Lactobacillus plantarum KKP/593/p, Lactobacillus plantarum KKP/788/p, Lactobacillus plantarum PCM B/00081, Lactobacillus plantarum (NCIMB 41638), Lactobacillus plantarum DSM 29024, Lactobacillus plantarum (ATCC 55943), Lactobacillus plantarum (ATCC 55944), Lactobacillus plantarum (ATCC PTSA-6139), Lactobacillus plantarum (CNCM 1-3235), Lactobacillus plantarum DSM 11672, Lactobacillus plantarum (DSM 12836), Lactobacillus plantarum (DSM 12837), Lactobacillus plantarum (DSM 16565), Lactobacillus plantarum (DSM 16568), Lactobacillus plantarum (DSM 18112), Lactobacillus plantarum (DSM 18113), Lactobacillus plantarum (DSM 18114), Lactobacillus plantarum (DSM 19457), Lactobacillus plantarum (DSM 21762), Lactobacillus plantarum (DSM 23375), Lactobacillus plantarum (DSM 29025), Lactobacillus plantarum (DSM 3676), Lactobacillus plantarum (DSM 3677), Lactobacillus plantarum (DSM 8862), Lactobacillus plantarum (DSM 8866), Lactobacillus plantarum (LMG-21295), Lactobacillus plantarum (NCIMB 30083), Lactobacillus plantarum (NCIMB 30084), Lactobacillus plantarum (NCIMB 30084), Lactobacillus plantarum (NCIMB 30084), Lactobacillus plantarum (NCIMB 30236), Lactobacillus plantarum (NCIMB 41028), Lactobacillus plantarum (NCIMB 42150), Lactobacillus plantarum (VTT E-78076), Lactobacillus plantarum C KKP/788/p, Lactobacillus plantarum CECT 4528, Lactobacillus plantarum CECT 4528, Lactobacillus plantarum K KKP/593/p, Lactobacillus plantarum LP287, Lactobacillus plantarum LP329, Lactobacillus plantarum LP329, or Lactobacillus plantarum NCIMB 30238.

In some embodiments wherein Rhodopseudomonas palustris comprises one or more microbial organisms of the microorganism consortium, illustrative strains may include, but are not limited to, comprising Rhodopseudomonas palustris Molisch van Niel ATCC 17001, Rhodopseudomonas palustris Molisch van Niel ATCC 33872, Rhodopseudomonas palustris Molisch van Niel ATCC 17005, Rhodopseudomonas palustris Molisch van Niel ATCC 17000, Rhodopseudomonas palustris Molisch van Niel ATCC BAA-1122, Rhodopseudomonas palustris Molisch van Niel ATCC 17010, Rhodopseudomonas palustris Molisch van Niel ATCC 17002, Rhodopseudomonas palustris Molisch van Niel ATCC 49781, Rhodopseudomonas palustris Molisch van Niel ATCC 17003, Rhodopseudomonas palustris Molisch van Niel ATCC 17004, Rhodopseudomonas palustris Molisch van Niel ATCC 51186, Rhodopseudomonas palustris Molisch van Niel ATCC 17006, Rhodopseudomonas palustris Molisch van Niel ATCC BAA-37, Rhodopseudomonas palustris Molisch van Niel ATCC 17008, Rhodopseudomonas palustris Molisch van Niel ATCC 17009, Rhodopseudomonas palustris Molisch van Niel ATCC BAA-98D-5, Rhodopseudomonas palustris Molisch van Niel ATCC BAA-1122D-5, Rhodopseudomonas palustris Molisch van Niel ATCC BAA-1123D-5, Rhodopseudomonas palustris Molisch van Niel ATCC BAA-98, Rhodopseudomonas palustris Molisch van Niel ATCC BAA-1123, Rhodopseudomonas palustris Molisch van Niel ATC BAA-1125, Rhodopseudomonas palustris Molisch van Niel ATCC 17007, Rhodopseudomonas palustris Molisch van Niel ATCC BAA-1124, or Rhodospirillum rubrum Esmarch Molisch ATCC 25852.

In some embodiments wherein Rhodopseudomonas sphaeroides comprises one or more microbial organisms of the microorganism consortium, illustrative strains may include, but are not limited to, Rhodobacter sphaeroides (van Niel) Imhoff et al. (ATCC 35055), Rhodobacter sphaeroides (van Niel) Imhoff et al. (ATCC 17025D-5), Rhodobacter sphaeroides (van Niel) Imhoff et al. (ATCC 17026), Rhodobacter sphaeroides (van Niel) Imhoff et al. (ATCC 17029D-5), Rhodobacter sphaeroides (van Niel) Imhoff et al. (ATCC 17023), Rhodobacter sphaeroides (van Niel) Imhoff et al. (ATCC 17029), Rhodobacter sphaeroides (van Niel) Imhoff et al. (ATCC 35055), Rhodobacter sphaeroides (van Niel) Imhoff et al. (ATCC 35053), Rhodobacter sphaeroides (van Niel) Imhoff et al. (ATCC 35054), Rhodobacter sphaeroides (van Niel) Imhoff et al. (ATCC 17025D-5), Rhodobacter sphaeroides (van Niel) Imhoff et al. (ATCC 17026), Rhodobacter sphaeroides (van Niel) Imhoff et al. (ATCC 17029D-5), Rhodobacter sphaeroides (van Niel) Imhoff et al. (ATCC 33575), Rhodobacter sphaeroides (van Niel) Imhoff et al. (ATCC 17023), Rhodobacter sphaeroides (van Niel) Imhoff et al. (ATCC 17027), Rhodobacter sphaeroides (van Niel) Imhoff et al. (ATCC 17028), Rhodobacter sphaeroides (van Niel) Imhoff et al. (ATCC 17024), Rhodobacter sphaeroides (van Niel) Imhoff et al. (ATCC 21286), Rhodobacter sphaeroides (van Niel) Imhoff et al. (ATCC 21455), and Rhodobacter sphaeroides NCIMB 8253.

In some embodiments wherein Saccharomyces cerevisiae comprises one or more microbial organisms of the microorganism consortium, illustrative strains may include, but are not limited to, Saccharomyces cerevisiae NCYC Sc47, Saccharomyces cerevisiae PCM KKP 2059p, Saccharomyces cerevisiae IFO 0203, Saccharomyces cerevisiae CNCM 1-3060, Saccharomyces cerevisiae NCYC R397, Saccharomyces cerevisiae CNCM 1-3399, Saccharomyces cerevisiae NCYC R646, Saccharomyces cerevisiae CBS 493.9, Saccharomyces cerevisiae CNCM 1-1077, Saccharomyces cerevisiae NCYC Sc 47, Saccharomyces cerevisiae CNCM 1-4407, Saccharomyces cerevisiae MUCL 39885, Saccharomyces cerevisiae NCYC R404, Saccharomyces cerevisiae NCYC R404, Saccharomyces cerevisiae PCM KKP 2059p, or Saccharomyces cerevisiae CNCM 1-1079.

In embodiments wherein Pichia kudriavzevii comprises one or more microbes of the microorganism consortium, illustrative strains may include, but are not limited to, Pichia occidentalis (Kurtzman et al.) Kurtzman et al. ATCC 28527, Candida krusei (Castellani) Berkhout ATCC 34135, Issatchenkia orientalis Kudrjanzev ATCC 24210, Candida krusei (Castellani) Berkhout ATCC 14243, Candida krusei (Castellani) Berkhout ATCC 28870, Issatchenkia orientalis Kudrjanzev ATCC 6258, Issatchenkia orientalis Kudrjanzev ATCC 6258, Komagataella pastoris (Guilliermond) Yamada et al. ATCC 28485, Komagataella pastoris (Guilliermond) Yamada et al. ATCC 28485D-5, Komagataella pastoris (Guilliermond) Yamada et al. ATCC 204163, Pichia occidentalis (Kurtzman et al.) Kurtzman et al. ATCC 28527, Candida krusei (Castellani) Berkhout ATCC 34135, Issatchenkia orientalis Kudrjanzev ATCC 24210, Candida krusei (Castellani) Berkhout ATCC 14243, Candida krusei (Castellani) Berkhout ATCC, Issatchenkia orientalis Kudrjanzev ATCC 6258, and Issatchenkia orientalis Kudrjanzev ATCC 6258.

In embodiments wherein Trichoderma virens comprises one or more microbes of the microorganism consortium, illustrative strains may include, but are not limited to, Trichoderma virens (Miller et al.) von Arx, anamorph ATCC 9645, Trichoderma virens (Miller et al.) von Arx, anamorph ATCC 13213, Trichoderma reesei Simmons ATCC 13631, Trichoderma reesei Simmons ATCC 13631D-2, Trichoderma virens (Miller et al.) von Arx, anamorph ATCC 9645D-2, Trichoderma virens (Miller et al.) von Arx, anamorph ATCC MYA-298, Trichoderma virens (Miller et al.) von Arx, anamorph ATCC MYA-650, Trichoderma virens (Miller et al.) von Arx, anamorph ATCC MYA-297, Trichoderma virens (Miller et al.) von Arx, anamorph ATCC 10043, Trichoderma virens (Miller et al.) von Arx, anamorph ATCC 10045, Trichoderma virens (Miller et al.) von Arx, anamorph ATCC 10044, Trichoderma virens (Miller et al.) von Arx, anamorph ATCC 204067, Trichoderma virens (Miller et al.) von Arx, anamorph ATCC 204444, Trichoderma virens (Miller et al.) von Arx, anamorph ATCC MYA-649, Trichoderma virens (Miller et al.) von Arx, anamorph ATCC 204443, Trichoderma virens (Miller et al.) von Arx, anamorph ATCC 204445, Trichoderma virens (Miller et al.) von Arx, anamorph ATCC MYA-651, Trichoderma virens (Miller et al.) von Arx, anamorph ATCC 58676, Trichoderma virens (Miller et al.) von Arx, anamorph ATCC 24290, Trichoderma virens (Miller et al.) von Arx, anamorph ATCC 52045, Trichoderma virens (Miller et al.) von Arx, anamorph ATCC 62399, Trichoderma virens (Miller et al.) von Arx, anamorph ATCC 52199, Trichoderma virens (Miller et al.) von Arx, anamorph ATCC 44734, Trichoderma virens (Miller et al.) von Arx, anamorph ATCC 64271, Trichoderma virens (Miller et al.) von Arx, anamorph ATCC 58677, Trichoderma virens (Miller et al.) von Arx, anamorph ATCC 44327, Trichoderma virens (Miller et al.) von Arx, anamorph ATCC 20904, Trichoderma virens (Miller et al.) von Arx, anamorph ATCC 20906, Trichoderma virens (Miller et al.) von Arx, anamorph ATCC 9645, Trichoderma virens (Miller et al.) von Arx, anamorph ATCC 48179, Trichoderma virens (Miller et al.) von Arx, anamorph ATCC 58678, Trichoderma virens (Miller et al.) von Arx, anamorph ATCC 13362, Trichoderma virens (Miller et al.) von Arx, anamorph ATCC 20903, Trichoderma virens (Miller et al.) von Arx, anamorph ATCC 42955, Trichoderma virens (Miller et al.) von Arx, anamorph ATCC 13213, Trichoderma vixens (Miller et al.) von Arx, anamorph ATCC MYA-4894, Trichoderma harzianum Rifai, anamorph ATCC 20671, Trichoderma harzianum Rifai, anamorph ATCC 204065, Trichoderma reesei Simmons ATCC 28217, Trichoderma virens (Miller et al.) von Arx, anamorph ATCC 204067, Trichoderma virens (Miller et al.) von Arx, anamorph ATCC 204444, Trichoderma virens (Miller et al.) von Arx, anamorph ATCC 58676, Trichoderma virens (Miller et al.) von Arx, anamorph ATCC 9645D-2, Trichoderma virens (Miller et al.) von Arx, anamorph ATCC MYA-298, Trichoderma virens (Miller et al.) von Arx, anamorph ATCC MYA-650, Trichoderma virens (Miller et al.) von Arx, anamorph ATCC MYA-4894, Trichoderma vixens (Miller et al.) von Arx, anamorph ATCC MYA-29, Trichoderma virens (Miller et al.) von Arx, anamorph ATCC 44734, Trichoderma vixens (Miller et al.) von Arx, anamorph ATCC MYA-649, Trichoderma virens (Miller et al.) von Arx, anamorph ATCC 64271, Trichoderma virens (Miller et al.) von Arx, anamorph ATCC 58677, Trichoderma virens (Miller et al.) von Arx, anamorph ATCC 20903, Trichoderma vixens (Miller et al.) von Arx, anamorph ATCC 204443, Trichoderma vixens (Miller et al.) von Arx, anamorph ATCC 42955, Trichoderma virens (Miller et al.) von Arx, anamorph ATCC 204445, Trichoderma vixens (Miller et al.) von Arx, anamorph ATCC MYA-651, Trichoderma virens (Miller et al.) von Arx, anamorph ATCC 10043, Trichoderma virens (Miller et al.) von Arx, anamorph ATCC 44327, Trichoderma virens (Miller et al.) von Arx, anamorph ATCC 24290, Trichoderma vixens (Miller et al.) von Arx, anamorph ATCC 10045, Trichoderma virens (Miller et al.) von Arx, anamorph ATCC 52045, Trichoderma virens (Miller et al.) von Arx, anamorph ATCC 20904, Trichoderma virens (Miller et al.) von Arx, anamorph ATCC 48179, Trichoderma vixens (Miller et al.) von Arx, anamorph ATCC 62399, Trichoderma virens (Miller et al.) von Arx, anamorph ATCC 58678, Trichoderma virens (Miller et al.) von Arx, anamorph ATCC 10044, Trichoderma virens (Miller et al.) von Arx, anamorph ATCC 13362, Trichoderma virens (Miller et al.) von Arx, anamorph ATCC 52199, and Trichoderma vixens (Miller et al.) von Arx, anamorph ATCC 20906.

In some embodiments, one or more microbial organisms of the microorganism consortium may comprise, but are not limited to, one or more of Bifidobacterium actinocoloniiforme, Bifidobacterium adolescentis, Bifidobacterium angulatum, Bifidobacterium animalis, Bifidobacterium aquikefiri, Bifidobacterium asteroides, Bifidobacterium biavatii, Bifidobacterium bifidum, Bifidobacterium bohemicum, Bifidobacterium bombi, Bifidobacterium boum, Bifidobacterium breve, Bifidobacterium callitrichos, Bifidobacterium catenulatum, Bifidobacterium choerinum, Bifidobacterium commune, Bifidobacterium coryneforme, Bifidobacterium cuniculi, Bifidobacterium crudilactis, Bifidobacterium denticolens, Bifidobacterium dentium, Bifidobacterium eulemuris, Bifidobacterium faecale, Bifidobacterium gallicum, Bifidobacterium gallinarum, Bifidobacterium hapali, Bifidobacterium indicum, Bifidobacterium inopinatum, Bifidobacterium kashiwanohense, Bifidobacterium infantis, Bifidobacterium lemurum, Bifidobacterium longum, Bifidobacterium magnum, Bifidobacterium merycicum, Bifidobacterium minimum, Bifidobacterium mongoliense, Bifidobacterium moukalabense, Bifidobacterium myosotis, Bifidobacterium pseudocatenulatum, Bifidobacterium pseudolongum, Bifidobacterium psychraerophilum, Bifidobacterium pullorum, Bifidobacterium reuteri, Bifidobacterium ruminantium, Bifidobacterium saguini, Bifidobacterium scardovii, Bifidobacterium stellenboschense, Bifidobacterium stercoris, Bifidobacterium saeculare, Bifidobacterium subtile, Bifidobacterium thermacidophilum, Bifidobacterium thermophilum, Bifidobacterium tissieri, and Bifidobacterium tsurumiense, Bifidobacterium animalis ssp. animalis (DSM 16284), Bifidobacterium longum subsp. Suis (DSM 20211), Bifidobacterium animalis subsp. animalis (DSM 20104), Bifidobacterium animalis subsp. lactis (DSM 20105), and Bifidobacterium animalis subsp. lactis (ATCC 27536), Lactobacillus acetotolerans, Lactobacillus acidifarinae, Lactobacillus acidipiscis, Lactobacillus acidophilus, Lactobacillus agilis, Lactobacillus algidus, Lactobacillus alimentarius, Lactobacillus amylolyticus, Lactobacillus amylophilus, Lactobacillus amylotrophicus, Lactobacillus amylovorus, Lactobacillus animalis, Lactobacillus antri, Lactobacillus apodemi, Lactobacillus aviarius, Lactobacillus bifermentans, Lactobacillus brevis, Lactobacillus buchneri, Lactobacillus camelliae, Lactobacillus casei, Lactobacillus catenaformis, Lactobacillus ceti, Lactobacillus coleohominis, Lactobacillus collinoides, Lactobacillus composti, Lactobacillus concavus, Lactobacillus coryniformis, Lactobacillus crispatus, Lactobacillus crustorum, Lactobacillus curvatus, Lactobacillus delbrueckii subsp. bulgaricus, Lactobacillus delbrueckii subsp. delbrueckii, Lactobacillus delbrueckii subsp. lactis, Lactobacillus dextrinicus, Lactobacillus diolivorans, Lactobacillus equi, Lactobacillus equigenerosi, Lactobacillus farraginis, Lactobacillus farciminis, Lactobacillus fermentum, Lactobacillus fornicalis, Lactobacillus fructivorans, Lactobacillus frumenti, Lactobacillus fuchuensis, Lactobacillus gallinarum, Lactobacillus gasseri, Lactobacillus gastricus, Lactobacillus ghanensis, Lactobacillus graminis, Lactobacillus hammesii, Lactobacillus hamsteri, Lactobacillus harbinensis, Lactobacillus hayakitensis, Lactobacillus helveticus, Lactobacillus hilgardii, Lactobacillus homohiochii, Lactobacillus iners, Lactobacillus ingluviei, Lactobacillus intestinalis, Lactobacillus jensenii, Lactobacillus johnsonii, Lactobacillus kalixensis, Lactobacillus kefiranofaciens, Lactobacillus kefiri, Lactobacillus kimchii, Lactobacillus kitasatonis, Lactobacillus kunkeei, Lactobacillus leichmannii, Lactobacillus lindneri, Lactobacillus malefermentans, Lactobacillus mali, Lactobacillus manihotivorans, Lactobacillus mindensis, Lactobacillus mucosae, Lactobacillus murinus, Lactobacillus nagelii, Lactobacillus namurensis, Lactobacillus nantensis, Lactobacillus oligofermentans, Lactobacillus oris, Lactobacillus panis, Lactobacillus pantheris, Lactobacillus parabrevis, Lactobacillus parabuchneri, Lactobacillus paracasei, Lactobacillus paracollinoides, Lactobacillus parafarraginis, Lactobacillus parakefiri, Lactobacillus paralimentarius, Lactobacillus paraplantarum, Lactobacillus pentosus, Lactobacillus perolens, Lactobacillus plantarum, Lactobacillus pontis, Lactobacillus protectus, Lactobacillus psittaci, Lactobacillus rennini, Lactobacillus reuteri, Lactobacillus rhamnosus, Lactobacillus rimae, Lactobacillus rogosae, Lactobacillus rossiae, Lactobacillus ruminis, Lactobacillus saerimneri, Lactobacillus sakei, Lactobacillus salivarius, Lactobacillus sanfranciscensis, Lactobacillus satsumensis, Lactobacillus secaliphilus, Lactobacillus sharpeae, Lactobacillus siliginis, Lactobacillus spicheri, Lactobacillus suebicus, Lactobacillus thailandensis, Lactobacillus ultunensis, Lactobacillus vaccinostercus, Lactobacillus vaginalis, Lactobacillus versmoldensis, Lactobacillus vini, Lactobacillus vitulinus, Lactobacillus zeae, and Lactobacillus zymae.

In some embodiments, the one or more microbial organisms of the microorganism consortium may include, but are not limited to, Lactobacillus buchneri KKP/907/p, Lactobacillus buchneri (DSM 22963), Lactobacillus buchneri (DSM 12856), Lactobacillus buchneri (DSM 13573), Lactobacillus buchneri CCM 1819, Lactobacillus buchneri (DSM 16774), Lactobacillus buchneri DSM 22501, Lactobacillus buchneri LN 40177, Lactobacillus buchneri LN4637, Lactobacillus buchneri LN 40177, Lactobacillus buchneri NCIMB 40788, Lactobacillus rhamnosus (NCIMB 41640), Lactobacillus rhamnosus (NCIMB 30121), Lactobacillus rhamnosus DSM 29226, Lactobacillus rhamnosus DSM 7133, Lactobacillus rhamnosus (CNCM-I-3698), Lactobacillus rhamnosus ATCC 7469, Lactobacillus fermentum (NCIMB 41636), Lactobacillus brevis (DSM 12835), Lactobacillus brevis (DSM 21982), Lactobacillus brevis (DSM 12835), Lactobacillus brevis DSMZ 16680, Lactococcus chungangensis, Lactococcus formosensis, Lactococcus fujiensis, Lactococcus garvieae, Lactococcus hircilactis, Lactococcus lactis, Lactococcus laudensis, Lactococcus nasutitermitis, Lactococcus piscium, Lactococcus plantarum, Lactococcus raffinolactis, and Lactococcus taiwanensis, Lactococcus lactis PCM B/00039, Lactococcus lactis (DSM 11037), Lactococcus lactis (NCIMB 30117), Lactococcus lactis (NCIMB 30160), Bacillus licheniformis, Bacillus amyloliquefaciens, Bacillus licheniformis DSM 28710, Bacillus licheniformis (DSM 19670), Bacillus licheniformis (DSM 21564), Bacillus licheniformis ATCC 53757, Bacillus amyloliquefaciens CECT 5940, Bacillus amyloliquefaciens (DSM 9553), Bacillus amyloliquefaciens (DSM 9554), Bacillus amyloliquefaciens (PTA-6507), Bacillus amyloliquefaciens (NRRL B-50013), Bacillus amyloliquefaciens (NRRL B-50104), Bacillus amyloliquefaciens SD80, Bacillus amyloliquefaciens (ATCC 3978), Carnobacterium alterfunditum, Carnobacterium divergens, Carnobacterium funditium, Carnobacterium gallinarum, Carnobacterium iners, Carnobacterium inhibens, Carnobacterium jeotgali, Carnobacterium maltaromaticum, Carnobacterium mobile, Carnobacterium piscicola, Carnobacterium pleistocenium, and Carnobacterium viridans, Carnobacterium divergens PCM KKP 2012p, Streptococcus thermophilus, Streptococcus thermophilus NBIMCC 8253, Streptococcus thermophilus CNRZ 1066, Streptococcus thermophilus LMG13811, Enterococcus faecium, Enterococcus faecalis, Enterococcus faecium CECT 4515, Enterococcus faecium CCM 6226, Enterococcus faecium CNCM 1-3236, Enterococcus faecium DSM 22502, Enterococcus faecium NCIMB 10415, Enterococcus faecium SF202, Enterococcus faecium SF301, Enterococcus faecium DSM 7134, Enterococcus faecium DSM 10663, Enterococcus faecium NCIMB 11181, Enterococcus faecium D SM 21913, and/or Enterococcus faecium NBIMCC 8270.

In some embodiments, the one or more microbial organisms of the microorganism consortium may include, but are not limited to, Alcaligenes sp., Aerobacter aerogenes, Achromobacter sp., Acinetobacter sp., Actinomadura oligospora, Agrobacterium sp., Azospirillum sp., Bacillus sp., Bacillus circulans, B. cereus, B. fusiformis, B. pumilis, B. megaterium, B. mycoides, B. polymyxa Paenibacillus polymyxa, B. coagulans, B. chitinolyticus Paenibacillus chitinolyticus, B. subtilis, Bacillus subtilis natto, Bradyrhizobium sp., Brevibacterium sp., Citrobacter sp., Pseudomonas sp., P putida, P. striata, P. fluorescens, P. calcis, Flavobacterium sp., Nitrosomonas sp., Erwinia sp., Micrococcus sp., Escherichia intermedia, Enterobacter asburiae, Serratia phosphoticum, Nitrobacter sp., Thiobacillus ferroxidans, T thioxidans, Rhizobium meliloti, Xanthomonas sp., Aspergillus awamori, A. niger, A. tereus, A. flavus, A. nidulans, A. foetidus, A. wentii. Fusarium oxysporum, Alternaria teneius, Achrothcium sp. Penicillium digitatum, P lilacinium, P balaji, P. funicolosum, Cephalosporium sp. Cladosprium sp., Curvularia lunata, Cunnighamella, Candida sp., Chaetomium globosum, Humicola inslens, Humicola lanuginosa, Helminthosporium sp., Paecilomyces fusisporous, Pythium sp., Phoma sp., Populospora mytilina, Myrothecium roridum, Morteirella sp., Micromonospora sp., Oideodendron sp., Rhizoctonia solani, Rhizopus sp., Mucor sp., Trichoderma viridae, Torula thermophila, Schwanniomyces occidentalis, Sclerotium rolfsii, Actinomyces, Streptomyces., Anabena sp., Calothrix braunii, Nostoc sp., Scytonema sp., or Glomus fasciculatum.

In some embodiments, the one or more microbial organisms of the microorganism consortium may include, but are not limited to, Lactobacillus rapi DSM-19907, Lactobacillus rapi Type Strain JCM 15042 NRIC 0743, Leuconostoc rapi DSM-27776, or Leuconostoc rapi Type Strain LMG 27676.

In some embodiments, the one or more microbial organisms of the microorganism consortium may include, but are not limited to, Alcaligenes faecalis ATCC 8750, Genomic DNA from Alcaligenes faecalis subsp. faecalis strain 16 ATCC 8750D-5, Alcaligenes faecalis subsp. faecalis Castellani and Chalmers ATCC 35655, Alcaligenes faecalis Castellani and Chalmers ATCC 53602, Alcaligenes faecalis subsp. faecalis Castellani and Chalmers ATCC 19018, Alcaligenes faecalis subsp. faecalis Castellani and Chalmers ATC 35655, Alcaligenes faecalis subsp. faecalis Castellani and Chalmers ATCC 27260, Alcaligenes faecalis subsp. faecalis Castellani and Chalmers ATCC 49677, Alcaligenes faecalis subsp. faecalis Castellani and Chalmers ATCC 33950, Alcaligenes faecalis subsp. faecalis Castellani and Chalmers ATCC 15554, Alcaligenes faecalis subsp. faecalis Castellani and Chalmers ATCC 43161, Alcaligenes faecalis Castellani and Chalmers ATCC 15101, Alcaligenes faecalis subsp. faecalis Castellani and Chalmers ATCC 8748, Alcaligenes faecalis subsp. faecalis Castellani and Chalmers ATCC 15246, Alcaligenes faecalis subsp. faecalis Castellani and Chalmers ATCC 19209, Alcaligenes faecalis Castellani and Chalmers ATCC 700596, or Alcaligenes faecalis subsp. faecalis Castellani and Chalmers ATCC 33585.

In some embodiments, the one or more microbial organisms of the microorganism consortium may include, but are not limited to, Achromobacter xylosoxidans ex Yabuuchi and Ohyama Yabuuchi and Yano ATCC 27061, Achromobacter xylosoxidans ex Yabuuchi and Ohyama Yabuuchi and Yano ATCC 212, Achromobacter xylosoxidans ex Yabuuchi and Ohyama Yabuuchi and Yano ATCC 27062, Achromobacter xylosoxidans ex Yabuuchi and Ohyama Yabuuchi and Yano ATCC 27063, Achromobacter xylosoxidans ex Yabuuchi and Ohyama Yabuuchi and Yano ATCC 31040, Achromobacter xylosoxidans ex Yabuuchi and Ohyama Yabuuchi and Yano ATCC 9220, Achromobacter xylosoxidans ex Yabuuchi and Ohyama Yabuuchi and Yano ATCC 15446, Achromobacter denitrificans Ruger and Tan Coenye et al. ATCC 15173, Achromobacter denitrificans Ruger and Tan Coenye et al. ATCC 55564, Achromobacter denitrificans Ruger and Tan Coenye et al. ATC 13138, Achromobacter piechaudii Kiredjian et al. Yabuuchi et al. ATC 43552, or Achromobacter ruhlandii Packer and Vishniac Yabuuchi et al. ATCC 15749.

In some embodiments, the one or more microbial organisms of the microorganism consortium may include, but are not limited to, Acinetobacter lwoffii Audureau Brisou ATCC 15309, Acinetobacter lwoffii Audureau Brisou ATCC 17925, Acinetobacter sp. ATCC 39769, Acinetobacter sp. ATCC 39770, Acinetobacter baumannii Bouvet and Grimont ATCC 19606, Acinetobacter baumannii Bouvet and Grimont ATCC 19606, Acinetobacter calcoaceticus Beijerinck Baumann et al. ATCC 23055, Acinetobacter sp. genomospecies 3 ATCC 19004, Acinetobacter junii Bouvet and Grimont ATCC 17908, Acinetobacter guillouiae Nemec et al. ATCC 11171, Acinetobacter bereziniae Nemec et al. ATCC 17924, Acinetobacter haemolyticus Bouvet and Grimont ATCC 17906, Acinetobacter johnsonii Bouvet and Grimont ATCC 17909, Acinetobacter sp. genomospecies 9 ATCC 17910, Acinetobacter sp. genomospecies 9 ATCC 17984, Acinetobacter sp. genomospecies 9 ATCC 17968, Acinetobacter sp. genomospecies 3 ATCC 17922, Acinetobacter sp. genomospecies 6 ATCC 17979, or Acinetobacter sp. genomospecies 9 ATCC 9957, Acinetobacter calcoaceticus Beijerinck Baumann et al. ATCC 23055, Acinetobacter calcoaceticus Beijerinck Baumann et al. ATCC 14987, Acinetobacter calcoaceticus Beijerinck Baumann et al. ATCC 17902, Acinetobacter calcoaceticus Beijerinck Baumann et al. ATCC 31926, Acinetobacter calcoaceticus Beijerinck Baumann et al. ATCC BAA-347, Acinetobacter baumannii Bouvet and Grimont ATCC 49466, Acinetobacter baylyi ATCC 33305, Acinetobacter baumannii ATCC 43498, pSC161RM O/P17 ATCC 40897, Acinetobacter sp. ATCC 39647, Acinetobacter baylyi ATCC 33304, Acinetobacter sp. ATCC 43608, Acinetobacter sp. ATCC 39648, Acinetobacter sp. ATCC 49823, Acinetobacter calcoaceticus Beijerinck Baumann et al. ATCC 19638, Acinetobacter baumannii Bouvet and Grimont ATCC 51432, Acinetobacter sp. ATCC 33969, Acinetobacter sp. ATCC 49467, Acinetobacter sp. ATCC 49468, Acinetobacter sp. ATC 31299, Acinetobacter calcoaceticus Beijerinck Baumann et al. ATCC BAA-346, or Acinetobacter sp. ATCC 33951.

In some embodiments, the one or more microbial organisms of the microorganism consortium may include, but are not limited to, Azospirillum brasilense Tarrand et al. ATCC 35213, Azospirillum brasilense Tarrand et al. ATCC 29729, Azospirillum brasilense Tarrand et al. ATCC 29710, Azospirillum brasilense Tarrand et al. ATCC 29711, Azospirillum brasilense Tarrand et al. ATCC 29145, Azospirillum brasilense Tarrand et al. ATCC 35212, Azospirillum lipoferum Beijerinck Tarrand et al. ATCC 29707, Azospirillum lipoferum Beijerinck Tarrand et al. ATCC 29709, Azospirillum lipoferum Beijerinck Tarrand et al. ATCC 29731, Azospirillum lipoferum Beijerinck Tarrand et al. ATCC 29708, or Azospirillum irakense Khammas et al. ATCC 51182.

In some embodiments, the one or more microbial organisms of the microorganism consortium may include, but are not limited to, Actinomadura sp. is Actinomadura oligospora Mertz and Yao ATCC 43269, or Bacillus circulans Jordan ATCC 19439,

In some embodiments, the one or more microbial organisms of the microorganism consortium may include, but are not limited to, Bacillus cereus Frankland and Frankland ATCC 14579, Bacillus cereus Frankland and Frankland ATCC 10876, Bacillus cereus Frankland and Frankland ATCC 13061, Bacillus cereus Frankland and Frankland ATCC 10876, Bacillus cereus Frankland and Frankland ATCC 11778, Bacillus cereus Frankland and Frankland ATCC BAA-1005, Bacillus cereus Frankland and Frankland ATCC 15816, Bacillus cereus Frankland and Frankland ATCC 15817, Bacillus cereus Frankland and Frankland ATCC BAA-512, Bacillus cereus Frankland and Frankland ATCC 23261, Bacillus cereus Frankland and Frankland ATCC 21772, Bacillus cereus Frankland and Frankland ATCC 21634, Bacillus cereus Frankland and Frankland ATCC 7004, Bacillus cereus Frankland and Frankland ATCC 19637, Bacillus cereus Frankland and Frankland ATCC 21182, Bacillus cereus Frankland and Frankland ATCC 7064, Bacillus cereus Frankland and Frankland ATCC 23260, Bacillus cereus Frankland and Frankland ATCC 4342, Bacillus cereus Frankland and Frankland ATCC 49063, Bacillus cereus Frankland and Frankland ATCC 31430, Bacillus cereus Frankland and Frankland ATCC 43881, Bacillus cereus Frankland and Frankland ATCC 49064, Bacillus cereus Frankland and Frankland ATCC 14603, Bacillus cereus Frankland and Frankland ATCC 55000, Bacillus cereus Frankland and Frankland ATCC 9592, Bacillus cereus Frankland and Frankland ATCC 12480, Bacillus cereus Frankland and Frankland ATCC 21366, Bacillus cereus Frankland and Frankland ATCC 10702, Bacillus cereus Frankland and Frankland ATCC 700282, Bacillus cereus Frankland and Frankland ATCC 33018, Bacillus cereus Frankland and Frankland ATCC 10987, Bacillus cereus Frankland and Frankland ATCC 53522, Bacillus cereus Frankland and Frankland ATCC 246, Bacillus cereus Frankland and Frankland ATCC 21769, Bacillus cereus Frankland and Frankland ATCC 9139, Bacillus cereus Frankland and Frankland ATCC 27522, Bacillus cereus Frankland and Frankland ATCC 33019, Bacillus cereus Frankland and Frankland ATCC 7039, Bacillus cereus Frankland and Frankland ATCC 55055, Bacillus cereus Frankland and Frankland ATCC 21768, Bacillus cereus Frankland and Frankland ATCC 27877, Bacillus cereus Frankland and Frankland ATCC 14737, Bacillus cereus Frankland and Frankland ATCC 55609, Bacillus cereus Frankland and Frankland ATCC 27348, Bacillus cereus Frankland and Frankland ATCC 9818, Bacillus cereus Frankland and Frankland ATCC 6464, Bacillus cereus Frankland and Frankland ATCC 25621, Bacillus cereus Frankland and Frankland ATCC 11950, Bacillus cereus Frankland and Frankland ATCC 14893, Bacillus cereus Frankland and Frankland ATCC 13472, Bacillus cereus Frankland and Frankland ATCC 13824, Bacillus cereus Frankland and Frankland ATCC 14579D-5, Bacillus cereus Frankland and Frankland ATCC 10987D-5, Bacillus cereus Frankland and Frankland ATCC 10702D-5, Bacillus cereus Frankland and Frankland ATCC 13061D-5, Bacillus cereus Frankland and Frankland ATCC 10876D-5, Bacteriophage of Bacillus cereus-anthracis, NikoA PTA-4171, Bacteriophage of Bacillus cereus-anthracis, DDBa PTA-4172, Bacteriophage of Bacillus cereus-anthracis, MHWa PTA-4173, or Bacillus cereus 21771.

In some embodiments, the one or more microbial organisms of the microorganism consortium may include, but are not limited to, Bacillus thuringiensis Berliner ATCC 10792 Strain Designations: [CCUG 7429, CIP 53.137, DSM 2046, HAMBI 478, LMG 7138, NCAIM B.01292, NCCB 70008, NRRL HD-735, VKM B-1544], Bacillus thuringiensis Berliner ATCC 19265, Bacillus thuringiensis Berliner ATCC 13367, Bacillus cereus Frankland and Frankland ATCC 21928, Bacillus thuringiensis Berliner ATCC 39152, or Bacillus thuringiensis Berliner ATCC 13366.

In some embodiments, the one or more microbial organisms of the microorganism consortium may include, but are not limited to, Bacillus weihenstephanensis Lechner et al. ATCC 12826, Bacillus weihenstephanensis bacteriophage ATCC 12826-B2, 31293, 31429, 31292, or Bacillus sp. ATCC 13062, P7 ATCC 75237.

In some embodiments, the one or more microbial organisms of the microorganism consortium may include, but are not limited to, Brevundimonas diminuta Leifson and Hugh Segers et al. ATCC 19146 or Brevundimonas diminuta Leifson and Hugh Segers et al. ATCC 19146.

In some embodiments, the one or more microbial organisms of the microorganism consortium may include, but are not limited to, Bacillus licheniformis Weigmann Chester ATCC 25972 or Bacillus thuringiensis Berliner ATCC 700872.

In some embodiments, the one or more microbial organisms of the microorganism consortium may include, but are not limited to, Bacillus pumilus Meyer and Gottheil ATCC 7061, Bacillus pumilus Meyer and Gottheil ATCC BAA-1434, Bacillus pumilus Meyer and Gottheil ATCC 700814, Bacillus pumilus Meyer and Gottheil ATCC 19646, Bacillus pumilus Meyer and Gottheil ATCC 14884, Bacillus pumilus Meyer and Gottheil ATCC 70, Bacillus pumilus Meyer and Gottheil ATCC 31650, Bacillus pumilus Meyer and Gottheil ATCC 98, Bacillus pumilus Meyer and Gottheil ATCC 71, Bacillus pumilus Meyer and Gottheil ATCC 19164, Bacillus pumilus Meyer and Gottheil ATCC 31095, Bacillus pumilus Meyer and Gottheil ATCC 4522, Bacillus pumilus Meyer and Gottheil ATCC 945, Bacillus pumilus Meyer and Gottheil ATCC 31093, Bacillus pumilus Meyer and Gottheil ATCC 21143, Bacillus pumilus Meyer and Gottheil ATCC 27142, Bacillus pumilus Meyer and Gottheil ATCC 6632, Bacillus pumilus Meyer and Gottheil ATCC 19878, Bacillus pumilus Meyer and Gottheil ATCC 31340, Bacillus pumilus Meyer and Gottheil ATCC 19546, Bacillus pumilus Meyer and Gottheil ATCC 7065, Bacillus pumilus Meyer and Gottheil ATCC 53206, Bacillus pumilus Meyer and Gottheil ATCC 4510, Bacillus pumilus Meyer and Gottheil ATCC 19548, Bacillus pumilus Meyer and Gottheil ATCC BAA-1434, Bacillus pumilus Meyer and Gottheil ATCC 31132, Bacillus pumilus Meyer and Gottheil ATCC 15716, Bacillus pumilus Meyer and Gottheil ATCC 19646, Bacillus pumilus Meyer and Gottheil ATCC 18, Bacillus pumilus Meyer and Gottheil ATCC 4520, Bacillus pumilus Meyer and Gottheil ATCC 6631, Bacillus pumilus Meyer and Gottheil ATCC 31177, Bacillus pumilus Meyer and Gottheil ATCC 1 Strain Designations: AMC [NRS 309], Bacillus pumilus 19547, Bacillus subtilis Ehrenberg Cohn ATCC 15477 Strain Designations: M-24-1, Bacillus subtilis Ehrenberg Cohn ATCC 31098, Bacillus subtilis Ehrenberg Cohn ATCC 21951, Bacillus subtilis Ehrenberg Cohn ATCC 21008, Bacillus pumilus Meyer and Gottheil ATCC 19182, Bacillus pumilus Meyer and Gottheil ATCC 72, Bacillus subtilis 21007, Bacillus subtilis 21358, Bacillus subtilis 21005, Bacillus subtilis 21006, Bacillus subtilis 21356, Bacillus subtilis 19220, 53683, Bacillus subtilis 19219, B. subtilis 53685, Bacillus subtilis 21357, B. subtilis 21398, B. subtilis 53689, Bacillus subtilis Ehrenberg Cohn ATCC 21770, or Bacillus subtilis 19217.

In some embodiments, the one or more microbial organisms of the microorganism consortium may include, but are not limited to, Bacillus mycoides Flugge ATCC 6462 Strain Designations: NRS 273 [155, CCUG 26678, CIP 103472, DSM 2048, HAMBI 1827, LMG 7128, NCTC 12974, NRRL B-14799, NRRL B-14811], Bacillus mycoides bacteriophage ATCC 11986-B1, Bacillus mycoides Flugge ATCC 31101, Bacillus mycoides Flugge ATCC 31103, Bacillus mycoides Flugge ATCC 10206, Bacillus mycoides Flugge ATCC 21929, Bacillus mycoides Flugge ATCC 11986, Bacillus mycoides Flugge ATCC 19647, Bacillus mycoides Flugge ATCC 31102, Rhodococcus rhodochrous Zopf Tsukamura emend. Rainey et al. ATCC 27, Bacillus cereus Frankland and Frankland ATCC 11778, Rhodococcus rhodochrous Zopf Tsukamura emend. Rainey et al. ATCC 999, Bacillus mycoides Flugge ATCC 6463, Bacillus mycoides Flugge ATCC 23258, Rhodococcus rhodochrous Zopf Tsukamura emend. Rainey et al. ATCC 4004, Bacillus mycoides Flugge ATCC 21929, or Bacillus pumilus Meyer and Gottheil ATCC 19646.

In some embodiments, the one or more microbial organisms of the microorganism consortium may include, but are not limited to, Bacillus coagulans Hammer ATCC 7050 Strain Designations: NRS 609 [NCIB 9365, NCTC 10334], Bacillus coagulans Hammer ATCC 12245 Strain Designations: [NCA 308], Bacillus coagulans Hammer ATCC 31284, Bacillus coagulans Hammer ATCC 53595, Bacillus coagulans Hammer ATCC 8038 Strain Designations: NCA 43P [NCIB 8080, NRS 770], Bacillus coagulans Hammer ATCC 15949 Strain Designations: NCA 4259, Bacillus coagulans Hammer ATCC 23498 Strain Designations: M-39, Bacillus coagulans Hammer ATCC 11369, Bacillus coagulans Hammer ATCC BAA-738, Bacillus coagulans Hammer ATCC 11014 Strain Designations: NRS T27 [78G], Bacillus coagulans Hammer ATCC 10545 Strain Designations: NRS 784 [NCIB 8041], pCR46 [Bacillus subtilis IS75] ATCC 67736, Bacillus cereus Frankland and Frankland ATCC 21366, Bacillus smithii Nakamura et al. ATCC 35670 Strain Designations: NRS 22 [815], Bacillus smithii Nakamura et al. ATCC 51232 Strain Designations: FRR B666 [SLS 37], or Bacillus cereus Frankland and Frankland ATCC 55055 Strain Designations: NEB 566.

In some embodiments, the one or more microbial organisms of the microorganism consortium may include, but are not limited to, Bacillus megaterium de Bary ATCC 14581 Strain Designations: [BCRC 10608, CCM 2007, CCUG 1817, CIP 66.20, DSM 32, HAMBI 2018, IAM 13418, JCM 2506, KCTC 3007, LMG 7127, NBRC 15308, NCCB 75016, NCIMB 9376, NCTC 10342, NRIC 1710, NRRL B-14308, VKM B-512], Bacillus megaterium de Bary ATCC 14581, Bacillus megaterium de Bary ATCC 13632, Bacillus megaterium de Bary ATCC 15117, Bacillus megaterium de Bary ATCC 7703, Bacillus megaterium de Bary ATCC 19135, Bacillus megaterium de Bary ATCC 33169, Bacillus megaterium de Bary pathovar cerealis ATCC 35075, Bacillus megaterium de Bary ATCC 25848, Bacillus megaterium de Bary ATCC 19213, Bacillus megaterium de Bary ATCC 6459, Bacillus megaterium de Bary ATCC 33166, Bacillus megaterium de Bary ATCC 21209, Bacillus megaterium de Bary ATCC 19160, Bacillus megaterium de Bary ATCC 33168, Bacillus megaterium de Bary ATCC 39118, Bacillus megaterium de Bary pathovar cerealis ATCC 35076, Bacillus megaterium de Bary ATCC 31294, Bacillus megaterium de Bary ATCC 13639, Bacillus megaterium de Bary ATCC 11478, Bacillus megaterium de Bary ATCC 33165, Bacillus megaterium de Bary ATCC 33729, Bacillus megaterium bacteriophage G ATCC 43725-B1, Bacillus megaterium de Bary ATCC 15451, Bacillus megaterium de Bary ATCC 7051, Bacillus megaterium de Bary ATCC 89, Bacillus megaterium de Bary ATCC 15374, Bacillus megaterium de Bary ATCC 33164, Bacillus megaterium de Bary ATCC 15127, Bacillus megaterium de Bary ATCC 15118, Bacillus megaterium de Bary ATCC 6458, Bacillus megaterium de Bary ATCC 12872, Bacillus megaterium de Bary ATCC 7052, Bacillus megaterium de Bary ATCC 21916, Bacillus megaterium de Bary ATCC 9885, Bacillus megaterium de Bary ATCC 33167, Bacillus megaterium de Bary ATCC 11562, Bacillus megaterium de Bary ATCC 25300, Bacillus megaterium de Bary ATCC 10778, Bacillus megaterium de Bary ATCC 13402, Bacillus megaterium de Bary ATCC 25833, Bacillus megaterium de Bary ATCC 21181, Bacillus megaterium de Bary ATCC 7056, Bacillus megaterium de Bary ATCC 4531, Bacillus megaterium de Bary ATCC 43725, Bacillus megaterium de Bary ATCC 27327, Bacillus megaterium de Bary ATCC 15781, Bacillus megaterium de Bary ATCC 49099, Bacillus megaterium de Bary ATCC 11561b, Bacillus megaterium de Bary ATCC 11561d, Bacillus megaterium de Bary ATCC 15450, Bacillus megaterium de Bary ATCC 49096, Bacillus megaterium de Bary ATCC 35985, Bacillus megaterium de Bary ATCC 14945, Bacillus megaterium de Bary ATCC 11561a, Bacillus megaterium de Bary ATCC 11561e, Bacillus megaterium de Bary ATCC 11561c, Bacillus megaterium de Bary ATCC 14946, Bacillus megaterium de Bary ATCC 11561, Bacillus megaterium 19161, Bacillus megaterium 15047, Bacillus megaterium 15046, Bacillus megaterium 19137, Bacillus megaterium 19218, Bacillus megaterium, SB 3112 PTA-3142, PTA-3142, Bacillus megaterium 15128, Bacillus megaterium 15177, Bacillus simplex Priest et al. ATCC 49097 Strain Designations: NRS 960 [DSM 1321], Bacillus flexus Priest et al. ATCC 49095 Strain Designations: NRS 665 [DSM 1320], Bacillus cereus Frankland and Frankland ATCC 55000 Strain Designations: B 153-2-2, Bacillus circulans Jordan ATCC 21737 Strain Designations: T5 [FERM-P 935], Bacillus simplex Priest et al. ATCC 13368 Strain Designations: 41, Bacillus sp. ATCC 13062 Strain Designations: MB-1073 [14-B22], Bacillus simplex Priest et al. ATCC 8011 Strain Designations: NRS 335 [NCTC 2597], Bacillus simplex Priest et al. ATCC 49098 Strain Designations: NRS 986 [DSM 1323], ATCC 19136, ATCC 39383, ATCC 39383, ATCC 21738, ATCC 21603, ATCC 19380, pBC16 ATCC 37338, ATCC 8245, or ATCC 51946.

In some embodiments, the one or more microbial organisms of the microorganism consortium may include, but are not limited to, Bacillus subtilis Ehrenberg Cohn ATCC 15245 Strain Designations: 3349 [IAM 1-3].

In some embodiments, the one or more microbial organisms of the microorganism consortium may include, but are not limited to, Enterobactor cloacae subsp. cloacae Jordan Hormaeche and Edwards, subsp. nov. ATCC 13047, Enterobacter cloacae subsp. dissolvens ATCC 23373, Enterobacter cloacae Jordan Hormaeche and Edwards ATCC BAA-2806, Enterobacter cloacae Jordan Hormaeche and Edwards ATCC BAA-2468, Enterobacter cloacae Jordan Hormaeche and Edwards ATCC BAA-2341, Enterobacter cloacae subsp. cloacae Jordan Hormaeche and Edwards, subsp. nov. ATCC 23355, Enterobacter cloacae subsp. cloacae Jordan Hormaeche and Edwards, subsp. nov. ATCC 15337, Enterobacter cloacae Jordan Hormaeche and Edwards ATCC BAA-2273, Enterobacter cloacae Jordan Hormaeche and Edwards ATCC BAA-2080, Enterobacter cloacae subsp. cloacae Jordan Hormaeche and Edwards, subsp. nov. ATCC 961, Enterobacter cloacae subsp. cloacae Jordan Hormaeche and Edwards, subsp. nov. ATCC 15361, Enterobacter cloacae subsp. cloacae Jordan Hormaeche and Edwards, subsp. nov. ATCC 13599, Enterobacter cloacae subsp. cloacae Jordan Hormaeche and Edwards, subsp. nov. ATCC 35589, Enterobacter cloacae subsp. cloacae Jordan Hormaeche and Edwards, subsp. nov. ATCC 35930, Enterobacter cloacae subsp. cloacae Jordan Hormaeche and Edwards, subsp. nov. ATCC 27508, Enterobacter cloacae Jordan Hormaeche and Edwards ATCC BAA-2357, Enterobacter cloacae subsp. cloacae Jordan Hormaeche and Edwards, subsp. nov. ATCC 19336, Enterobacter cloacae subsp. cloacae Jordan Hormaeche and Edwards, subsp. nov. ATCC 29893, Enterobacter cloacae subsp. cloacae Jordan Hormaeche and Edwards, subsp. nov. ATCC 39978, Enterobacter cloacae subsp. cloacae Jordan Hormaeche and Edwards, subsp. nov. ATCC 35549, Enterobacter cloacae subsp. cloacae Jordan Hormaeche and Edwards, subsp. nov. ATCC 10699, Enterobacter cloacae subsp. cloacae Jordan Hormaeche and Edwards, subsp. nov. ATCC 7256, Enterobacter cloacae subsp. cloacae Jordan Hormaeche and Edwards, subsp. nov. ATCC 35929, Enterobacter cloacae subsp. cloacae Jordan Hormaeche and Edwards, subsp. nov. ATCC 27613, Enterobacter cloacae subsp. cloacae Jordan Hormaeche and Edwards, subsp. nov. ATCC 35587, Enterobacter cloacae subsp. cloacae Jordan Hormaeche and Edwards, subsp. nov. ATCC 35592, Enterobacter cloacae subsp. cloacae Jordan Hormaeche and Edwards, subsp. nov. ATCC 12666, Enterobacter cloacae subsp. cloacae Jordan Hormaeche and Edwards, subsp. nov. ATCC 700644, Enterobacter cloacae subsp. cloacae Jordan Hormaeche and Edwards, subsp. nov. ATCC BAA-1143, Enterobacter cloacae subsp. cloacae Jordan Hormaeche and Edwards, subsp. nov. ATCC 35030, Enterobacter cloacae subsp. cloacae Jordan Hormaeche and Edwards, subsp. nov. ATCC 35591, Enterobacter cloacae Jordan Hormaeche and Edwards ATCC BAA-2271, Enterobacter cloacae subsp. cloacae Jordan Hormaeche and Edwards, subsp. nov. ATCC 962, Enterobacter cloacae subsp. cloacae Jordan Hormaeche and Edwards, subsp. nov. ATCC 39979, Enterobacter cloacae subsp. cloacae Jordan Hormaeche and Edwards, subsp. nov. ATC 29006, Enterobacter cloacae subsp. cloacae Jordan Hormaeche and Edwards, subsp. nov. ATCC 700411, Enterobacter cloacae Jordan Hormaeche and Edwards ATCC BAA-2272, Enterobacter cloacae subsp. cloacae Jordan Hormaeche and Edwards, subsp. nov. ATCC 529, Enterobacter cloacae subsp. cloacae Jordan Hormaeche and Edwards, subsp. nov. ATCC 13047D-5, Enterobacter cloacae subsp. dissolvens ATCC 23373D-5, Enterobacter cloacae PTA-3882, Enterobacter hormaechei O'Hara et al. ATCC 700323, Enterobacter hormaechei O'Hara et al. ATC 700323, Cronobacter sakazakii ATCC 29004, Enterobacter cloacae subsp. Cloacae ATCC 700621, Enterobacter cloacae subsp. cloacae Jordan Hormaeche and Edwards, subsp. nov. ATCC 29941, Enterobacter cloacae subsp. cloacae Jordan Hormaeche and Edwards, subsp. nov. ATCC 43091, Klebsiella aerogenes Tindall et al. ATCC BAA-2358, Enterobacter amnigenus Izard et al. ATC 51816, Enterobacter cloacae subsp. cloacae Jordan Hormaeche and Edwards, subsp. nov. ATCC 35590, Enterobacter cloacae subsp. cloacae Jordan Hormaeche and Edwards, subsp. nov. ATCC 29005, Enterobacter cloacae subsp. cloacae Jordan Hormaeche and Edwards, subsp. nov. ATCC 29249, Klebsiella pneumoniae subsp. pneumoniae Schroeter Trevisan ATC 27889, Enterobacter cloacae subsp. cloacae Jordan Hormaeche and Edwards, subsp. nov. ATCC 222, Enterobacter cloacae subsp. cloacae Jordan Hormaeche and Edwards, subsp. nov. ATCC 33457, Enterobacter cloacae subsp. cloacae Jordan Hormaeche and Edwards, subsp. nov. ATCC 43560, Enterobacter cloacae subsp. cloacae Jordan Hormaeche and Edwards, subsp. nov. ATCC 49141, Enterobacter cloacae subsp. cloacae Jordan Hormaeche and Edwards, subsp. nov. ATC 35588, Enterobacter cloacae subsp. cloacae Jordan Hormaeche and Edwards, subsp. nov. ATCC 700258, Enterobacter amnigenus Izard et al. ATCC 33072, or Enterobacter kobei Kosako et al. ATCC BAA-260.

In some embodiments, the one or more microbial organisms of the microorganism consortium may include, but are not limited to, Escherichia coli Migula Castellani and Chalmers ATCC 8739, Escherichia coli Migula Castellani and Chalmers ATCC 25922, Escherichia coli Migula Castellani and Chalmers ATCC 8739, Escherichia coli Migula Castellani and Chalmers ATCC CRM-8739, Escherichia coli Migula Castellani and Chalmers ATCC 25922 FDA strain Seattle 1946 [DSM 1103, NCIB 12210] Antigenic Properties Serotype 06, Biotype 1, Bacillus sp. ATCC BAA-1380, pJKK3-1 ATCC 37220, pBC16 ATCC 37338, Bacillus sp. ATCC 51912, ATCC 202074, ATCC 202075, or ATCC 202076.

In some embodiments, the one or more microbial organisms of the microorganism consortium may include, but are not limited to, Klebsiella aerogenes Tindall et al. ATCC 13048, Klebsiella aerogenes Tindall et al. ATCC 13048, Klebsiella aerogenes Tindall et al. ATCC 51697, Klebsiella aerogenes Tindall et al. ATCC 29008, Klebsiella aerogenes Tindall et al. ATCC 49701, Klebsiella aerogenes Tindall et al. ATCC 43175, Klebsiella aerogenes Tindall et al. ATCC 35028, Klebsiella aerogenes Tindall et al. ATC 29751, Klebsiella aerogenes Tindall et al. ATCC 15038, Klebsiella aerogenes Tindall et al. ATC BAA-2358, Klebsiella aerogenes Tindall et al. ATCC 49469, Klebsiella aerogenes Tindall et al. ATCC BAA-2347, Klebsiella aerogenes Tindall et al. ATCC 29010, Klebsiella aerogenes Tindall et al. ATCC BAA-2356, Klebsiella aerogenes Tindall et al. ATC 51342, Klebsiella aerogenes Tindall et al. ATCC 29007, Klebsiella aerogenes Tindall et al. ATCC 35029, Klebsiella aerogenes Tindall et al. ATCC 15038D-5, Klebsiella pneumoniae subsp. pneumoniae Schroeter Trevisan ATC 13882, Klebsiella pneumoniae subsp. pneumoniae Schroeter Trevisan ATCC 15380, Klebsiella oxytoca Flugge Lautrop ATC 15328, pSCH129 ATC 87423, Klebsiella pneumoniae subsp. pneumoniae Schroeter Trevisan ATCC 29519, Klebsiella pneumoniae subsp. pneumoniae Schroeter Trevisan ATCC 29517, Klebsiella aerogenes Tindall et al. ATCC 29009, Klebsiella pneumoniae subsp. pneumoniae Schroeter Trevisan ATCC 29518, Klebsiella pneumoniae subsp. pneumoniae Schroeter Trevisan ATCC 23357, Klebsiella oxytoca Flugge Lautrop ATCC 8724, Klebsiella pneumoniae subsp. pneumoniae Schroeter Trevisan ATC 13906, Klebsiella pneumoniae subsp. pneumoniae Schroeter Trevisan ATCC 13797, Klebsiella pneumoniae subsp. pneumoniae Schroeter Trevisan ATCC 23356, Klebsiella pneumoniae subsp. pneumoniae Schroeter Trevisan ATCC 25304, Klebsiella pneumoniae subsp. pneumoniae Schroeter Trevisan ATCC 9621, Klebsiella oxytoca Flugge Lautrop ATCC 12833, Klebsiella pneumoniae subsp. pneumoniae Schroeter Trevisan ATCC 25306, Klebsiella pneumoniae subsp. pneumoniae Schroeter Trevisan ATCC 12658, Klebsiella pneumoniae subsp. pneumoniae Schroeter Trevisan ATCC 21217, Klebsiella pneumoniae subsp. pneumoniae Schroeter Trevisan ATCC 21214, Klebsiella pneumoniae subsp. pneumoniae Schroeter Trevisan ATCC 21204, Klebsiella pneumoniae subsp. pneumoniae Schroeter Trevisan ATCC 25305, Klebsiella pneumoniae subsp. pneumoniae Schroeter Trevisan ATCC 211, Klebsiella pneumoniae subsp. pneumoniae Schroeter Trevisan ATC 12657, Klebsiella pneumoniae subsp. pneumoniae Schroeter Trevisan ATCC 21215, Klebsiella pneumoniae subsp. pneumoniae Schroeter Trevisan ATCC 27727, Klebsiella pneumoniae subsp. pneumoniae Schroeter Trevisan ATCC 27858, Klebsiella pneumoniae subsp. pneumoniae Schroeter Trevisan ATCC 25955, Klebsiella pneumoniae subsp. pneumoniae Schroeter Trevisan ATCC 8308, Klebsiella pneumoniae subsp. pneumoniae Schroeter Trevisan ATCC 21203, Klebsiella pneumoniae subsp. pneumoniae Schroeter Trevisan ATCC 21205, Klebsiella pneumoniae subsp. pneumoniae 21216, Klebsiella pneumoniae subsp. pneumoniae 21316, pMMB66EH ATCC 37620, pMMB67HE ATCC 37623, pJAK14 [JK388] ATCC 77289, pJAK16 [JK396] ATCC 77291, ATCC 77288, pJAK12 [JK386] ATCC 77287, pJAK17 [JK397] ATCC 77292, pMMB67EH ATCC 37622, pJAK15 [JK389] ATCC 77290, or pMMB66HE ATCC 37621.

In some embodiments, the one or more microbial organisms of the microorganism consortium may include, but are not limited to, Rhodopseudomonas palustris Molisch van Niel ATCC 17001, Rhodopseudomonas palustris Molisch van Niel ATCC 33872, Rhodopseudomonas palustris Molisch van Niel ATCC 17005, Rhodopseudomonas palustris Molisch van Niel ATCC 17000, Rhodopseudomonas palustris Molisch van Niel ATCC BAA-1122, Rhodopseudomonas palustris Molisch van Niel ATCC 17010, Rhodopseudomonas palustris Molisch van Niel ATCC 17002, Rhodopseudomonas palustris Molisch van Niel ATCC 49781, Rhodopseudomonas palustris Molisch van Niel ATCC 17003, Rhodopseudomonas palustris Molisch van Niel ATCC 17004, Rhodopseudomonas palustris Molisch van Niel ATCC 51186, Rhodopseudomonas palustris Molisch van Niel ATCC 17006, Rhodopseudomonas palustris Molisch van Niel ATCC BAA-37, Rhodopseudomonas palustris Molisch van Niel ATCC 17008, Rhodopseudomonas palustris Molisch van Niel ATCC 17009, Rhodopseudomonas palustris Molisch van Niel ATCC BAA-98D-5, Rhodopseudomonas palustris Molisch van Niel ATCC BAA-1122D-5, Rhodopseudomonas palustris Molisch van Niel ATCC BAA-1123D-5, Rhodopseudomonas palustris Molisch van Niel ATCC BAA-98, Rhodopseudomonas palustris Molisch van Niel ATCC BAA-1123, Rhodopseudomonas palustris Molisch van Niel ATC BAA-1125, Rhodopseudomonas palustris Molisch van Niel ATCC 17007, Rhodopseudomonas palustris Molisch van Niel ATCC BAA-1124, or Rhodospirillum rubrum Esmarch Molisch ATCC 25852.

In some embodiments, the one or more microbial organisms of the microorganism consortium may include, but are not limited to, Rhodobacter sphaeroides van Niel Imhoff et al. ATC 17023, Rhodobacter sphaeroides van Niel Imhoff et al. ATCC 49419, Rhodobacter sphaeroides van Niel Imhoff et al. ATCC 17024, Rhodobacter sphaeroides van Niel Imhoff et al. ATCC 17026, Rhodobacter sphaeroides van Niel Imhoff et al. ATCC 21455, Rhodobacter sphaeroides van Niel Imhoff et al. ATCC BAA-808, Rhodobacter sphaeroides van Niel Imhoff et al. ATCC 35055, Rhodobacter sphaeroides van Niel Imhoff et al. ATCC 35053, Rhodobacter sphaeroides van Niel Imhoff et al. ATCC 35054, Rhodobacter sphaeroides van Niel Imhoff et al. ATCC 17023D-5, Rhodobacter sphaeroides van Niel Imhoff et al. ATCC BAA-808D-5, Rhodobacter sphaeroides van Niel Imhoff et al. ATCC 17025D-5, Rhodobacter sphaeroides van Niel Imhoff et al. ATCC 17029D-5, Rubrivivax sp. ATCC 55304, Rhodobacter sphaeroides van Niel Imhoff et al. ATC 17028, Rhodobacter sphaeroides van Niel Imhoff et al. ATCC 17029, Rhodobacter sphaeroides van Niel Imhoff et al. ATCC 17027, Rhodobacter sphaeroides van Niel Imhoff et al. ATCC 21286, Rhodobacter sphaeroides van Niel Imhoff et al. ATCC 33575, Paracoccus denitrificans Beijerinck and Minkman Davis emend. Rainey et al. ATCC 17741 Strain Designations: 381 [CIP 106306, CIP 106400, DSM 413, IAM 12479, ICPB 3979, IFO 16712, JCM 6892, LIVID 22.21, LMG 4218, NCCB 22021, NCIB 11627, VKM B-1324], or Rhodobacter azotoformans ATC 17025.

In some embodiments, the one or more microbial organisms of the microorganism consortium may include, but are not limited to, Paenibacillus polymyxa Prazmowski Ash et al. ATCC 21830, Paenibacillus polymyxa Prazmowski Ash et al. ATCC 842, Paenibacillus polymyxa Prazmowski Ash et al. ATCC 7070, Paenibacillus polymyxa Prazmowski Ash et al. ATCC 43865, Paenibacillus polymyxa Prazmowski Ash et al. ATCC 12060 Strain Designations: CN 2222, Paenibacillus polymyxa Prazmowski Ash et al. ATCC 8525 Strain Designations: [NRS 813], Paenibacillus polymyxa Prazmowski Ash et al. ATCC 12321 Strain Designations: NRRL B-510 RHG, Paenibacillus polymyxa Prazmowski Ash et al. ATCC 8524 Strain Designations: [NRS 280; 8278], Paenibacillus polymyxa Prazmowski Ash et al. ATCC 7047 Strain Designations: NRS 251, Bacillus circulans Jordan ATCC 31228 Strain Designations: SC 10275, Paenibacillus polymyxa Prazmowski Ash et al. ATCC 8519 Strain Designations: [ATCC 9825, NRS 812], Paenibacillus polymyxa Prazmowski Ash et al. ATCC 8523 Strain Designations: [NRS 354], Paenibacillus polymyxa Prazmowski Ash et al. ATCC 39564 Strain Designations: 9A, Paenibacillus polymyxa Prazmowski Ash et al. ATCC 25901 Strain Designations: 63, Paenibacillus polymyxa Prazmowski Ash et al. ATCC 8526 Strain Designations: [NRS 297], Paenibacillus polymyxa Prazmowski Ash et al. ATCC 7070 Strain Designations: [Difco Labs. 8277, N.R. Smith NRS 279, Vitek #200115], Paenibacillus polymyxa Prazmowski Ash et al. ATCC 10401 Strain Designations: [BUCSAV 163, Boots 163, CCM 1460, CCM 1461, CCM 1465, NCDO 731, NCIB 8094, NCTC 1380, NRS 2010], Paenibacillus polymyxa Prazmowski Ash et al. ATCC 27955 Strain Designations: M4, Paenibacillus polymyxa Prazmowski Ash et al. ATCC 21551 Strain Designations: FH-K 890/3, Paenibacillus polymyxa Prazmowski Ash et al. ATCC 31037 Strain Designations: BN-109 [FERM-P 2129], Paenibacillus polymyxa Prazmowski Ash et al. ATCC 12712 Strain Designations: PRL B505, Paenibacillus polymyxa Prazmowski Ash et al. ATCC 21993 Strain Designations: 3-8 [FERM-P 412],

In some embodiments, the one or more microbial organisms of the microorganism consortium may include, but are not limited to, Paenibacillus chitinolyticus NRRL B-23119, Paenibacillus macerans Schardinger Ash et al. ATCC 8244 Strain Designations: NRS 888 [NCIB 9368, NCTC 6355], Paenibacillus macerans Schardinger Ash et al. ATCC 8509 Strain Designations: 18 [NRS 1095, Vitek #200237], Paenibacillus macerans Schardinger Ash et al. ATCC 7069 Strain Designations: NRS 277 [8275], Paenibacillus macerans Schardinger Ash et al. ATCC 8510 Strain Designations: 2037 [NRS 1096], Paenibacillus macerans Schardinger Ash et al. ATCC 8517 Strain Designations: 583 [NRS 1101], Paenibacillus macerans Schardinger Ash et al. ATCC 843 Strain Designations: [NRS 1093], Paenibacillus macerans Schardinger Ash et al. ATCC 8514 Strain Designations: 588 [NRS 1098], Paenibacillus macerans Schardinger Ash et al. ATCC 7048 Strain Designations: NRS 649 [ATCC 8512], Paenibacillus macerans Schardinger Ash et al. ATCC 8513 Strain Designations: 577 [NRS 1097], Paenibacillus macerans Schardinger Ash et al. ATCC 8509 Strain Designations: 18 [NRS 1095, Vitek #200237], Paenibacillus macerans Schardinger Ash et al. ATCC 8516 Strain Designations: 585 [NRS 1100], Paenibacillus macerans Schardinger Ash et al. ATCC 7068 Strain Designations: NRS 278 [8276], Paenibacillus macerans Schardinger Ash et al. ATCC 8518 Strain Designations: [NRS 646], Paenibacillus macerans Schardinger Ash et al. ATCC 8515 Strain Designations: 573 [NRS 1099], Paenibacillus macerans Pma 007 BR-97 202152, or Paenibacillus graminis Berge et al. ATCC 49035 Strain Designations: NRRL B-390 [NRS 373].

In some embodiments, the one or more microbial organisms of the microorganism consortium may include, but are not limited to, Pseudomonas putida Trevisan Migula ATCC 15070, Pseudomonas putida NRRL B-8, Pseudomonas putida NRRL B-13, Pseudomonas putida NRRL B-21, Pseudomonas putida NRRL B-22, Pseudomonas putida NRRL B-251, Pseudomonas putida NRRL B-252, Pseudomonas putida NRRL B-254, Pseudomonas putida NRRL B-723, Pseudomonas putida NRRL B-805, Pseudomonas putida NRRL B-993, Pseudomonas putida NRRL B-1023, Pseudomonas putida NRRL B-1245, Pseudomonas putida NRRL B-1486, Pseudomonas putida NRRL B-1595, Pseudomonas putida NRRL B-2084, Pseudomonas putida NRRL B-2267, Pseudomonas putida NRRL B-2459, Pseudomonas putida NRRL B-2914, Pseudomonas putida NRRL B-4067, Pseudomonas putida NRRL B-1468, Pseudomonas putida NRRL B-14875, Pseudomonas putida NRRL B-14878, Pseudomonas putida NRRL B-14887, Pseudomonas putida NRRL B-14888, Pseudomonas putida NRRL B-14938, Pseudomonas putida NRRL B-23263, Pseudomonas putida NRRL B-41390, Pseudomonas putida NRRL B-2084 Pseudomonas striata, Pseudomonas putida NRRL B-2914, or Pseudomonas striata.

In some embodiments, the one or more microbial organisms of the microorganism consortium may include, but are not limited to, Pseudomonas fluorescens Migula ATCC 13525 Strain Designations: NCTC 10038 [28/5, CCEB 546, DSM 50090, NCIB 9046, NCPPB 1964, PJ239, R. Hugh 818, R.Y. Stanier 192, Biotype A], Pseudomonas fluorescens Migula ATCC 17400 Strain Designations: 18 [IFO 15833, WRRL P-7], Pseudomonas fluorescens Migula ATCC 17556 Strain Designations: 188 [NCPPB 316 Pseudomonas marginata, PJ 160], Pseudomonas fluorescens Migula ATCC 17550 Strain Designations: 182 [IFO 3081, PJ 73], Pseudomonas fluorescens Migula ATCC 17555 Strain Designations: 187 [NCPPB 263 Pseudomonas angulata, PJ 139], Pseudomonas fluorescens Migula ATCC BAA-1781 Strain Designations: CNG89, Pseudomonas fluorescens Migula ATCC 31732, Pseudomonas fluorescens Migula ATCC 53795, Pseudomonas fluorescens Migula ATCC 13459 Strain Designations: PW2 [ICMP 3966, NCPPB 967, NRRL B-899], Pseudomonas fluorescens Migula ATCC 15917 Strain Designations: IAM 1055 [AHH-23], Pseudomonas fluorescens Migula ATCC 43203, Pseudomonas fluorescens Migula ATCC 33512 Strain Designations: 72, Pseudomonas fluorescens Migula ATCC 15456 Strain Designations: CCEB 488-A [BU 140], Pseudomonas fluorescens Migula ATCC 11253 Strain Designations: [NRRL B-1244], Pseudomonas fluorescens Migula ATCC 39502 Strain Designations: SC 12936, Pseudomonasfluorescens Migula ATCC 17554 Strain Designations: 186 [IFO 15829, PJ 79], Pseudomonas fluorescens Migula ATCC 700281 Strain Designations: H13, Pseudomonas fluorescens Migula ATCC 21541 Strain Designations: 30-21, Pseudomonas fluorescens Migula Strain Designations: 1291 [ATCC 17458, IFO 15837, NCIB 8917; LA, NRRL B-1864, R.Y. Stanier 83], Pseudomonas fluorescens Migula ATCC 17513 Strain Designations: 143 [IFO 15836, PL], Pseudomonas fluorescens Migula ATCC 17482 Strain Designations: 108 [52-22, IFO 15832], Pseudomonas fluorescens Migula ATCC 31950 Strain Designations: A505 [A5-05-1], Pseudomonas fluorescens Migula ATCC 12843 Strain Designations: NCIB 8866 strain CO2, Pseudomonas fluorescens Migula ATCC 27663 Strain Designations: PW, Pseudomonas fluorescens Migula ATCC 17574 Strain Designations: 207 [PJ 693], Pseudomonas fluorescens Migula ATCC 15453 Strain Designations: 52-1C, Pseudomonasfluorescens Migula ATCC 49667 Strain Designations: F-12, Pseudomonas fluorescens Migula ATCC 17467 Strain Designations: 93 [TR-10], Pseudomonas fluorescens Migula ATCC 17569 Strain Designations: 202 [PJ 372], Pseudomonas fluorescens Migula ATCC 15553 Strain Designations: CCEB 553 [IEM 15/47], Pseudomonas fluorescens Migula ATCC 31419 Strain Designations: IAM-1126 [43F], Pseudomonas fluorescens Migula ATCC 17565 Strain Designations: 198 [PJ 302], Pseudomonas fluorescens Migula ATCC 17575 Strain Designations: 208 [PJ 722], Pseudomonas fluorescens Migula ATCC 49270 Strain Designations: F 1847 [CDC-EB], Pseudomonas fluorescens Migula ATCC 17579 Strain Designations: 212 [PJ 832], Pseudomonas fluorescens Migula ATCC 17563 Strain Designations: 196 [PJ 288], Pseudomonas fluorescens Migula ATCC 13430 Strain Designations: Pyrrolidine, Pseudomonas fluorescens Migula ATCC 17557 Strain Designations: 189 [PJ227; 1208], Pseudomonas fluorescens Migula ATCC 17518 Strain Designations: 149 [2-40-40, IFO 15838], Pseudomonas fluorescens Migula ATCC 13475, Pseudomonas fluorescens Migula ATCC 17570 Strain Designations: 203 [PJ 376], Pseudomonas fluorescens Migula ATCC 949 Strain Designations: 1062, Pseudomonas fluorescens Migula ATCC 23728, Pseudomonas fluorescens Migula ATCC 17573 Strain Designations: 206 [PJ 692], Pseudomonas fluorescens Migula ATCC 17559 Strain Designations: 191 [IFO 15834, PJ 236; 22/1], Pseudomonas fluorescens Migula ATCC 17561 Strain Designations: 194 [Klinge R-60, PJ 253], Pseudomonas fluorescens Migula ATCC 35858 Strain Designations: NRRL B-4290, Pseudomonas fluorescens Migula ATCC 17582 Strain Designations: 215 [PJ 849], Pseudomonasfluorescens Migula ATCC 6972 Strain Designations: NCTC 2583 [NCIB 8194], Pseudomonas fluorescens Migula ATCC 17553 Strain Designations: 185 [W2 L-1], Pseudomonas fluorescens Migula ATCC 17571 Strain Designations: 204 [IFO 15835, PJ 682], Pseudomonas fluorescens Migula ATCC 55129 Strain Designations: SC 15208, Pseudomonas fluorescens bacteriophage phi-S1 ATCC 27663-B1 Strain Designations: Phi-S1, Pseudomonasfluorescens Migula ATCC 700830, Pseudomonasfluorescens Migula ATCC 53794 Strain Designations: PRA25, Pseudomonas fluorescens Migula ATCC 17552 Strain Designations: 184 [IFO 15830], Pseudomonas fluorescens Migula ATCC 17572 Strain Designations: 205 [PJ 686], Pseudomonas fluorescens Migula ATCC 23611, Pseudomonas fluorescens Migula ATCC 31125 Strain Designations: P-2563 [FERM-P 2894, IFO 13658], Pseudomonas fluorescens Migula ATCC 25289 Strain Designations: PYR, Pseudomonas fluorescens Migula ATCC 17634 Strain Designations: 267 [B-9], Pseudomonas fluorescens Migula ATCC 55241 Strain Designations: BNL-WVC, Pseudomonas fluorescens Migula ATCC 11150 Strain Designations: [NCIB 8286], Pseudomonas fluorescens Migula ATCC 49656, Pseudomonas fluorescens Migula ATCC 17568 Strain Designations: 201 [PJ 368], Pseudomonas fluorescens Migula ATCC 15916 Strain Designations: JAM 1008 [AHH-27], Pseudomonas fluorescens Migula ATCC 948 Strain Designations: 1013 [ATCC 11251, CCEB 295, IR) 3903, Jessen PJ 70, KY 3975, NCIB 9493, NRRL B-10, Stanier 181], Pseudomonasfluorescens Migula ATCC 14150 Strain Designations: NRRL B-1603 [6, IFO 15840], Pseudomonas fluorescens Migula ATCC 53859 Strain Designations: 6133D02, Pseudomonas fluorescens 53958, Pseudomonas fluorescens CGA 270294 55174, Pseudomonas fluorescens CGA 270293 55175, Pseudomonas fluorescens CGA 281836 55168, Pseudomonas fluorescens biovar I: MB214 PTA-7840, Pseudomonas fluorescens 21256, Pseudomonas fluorescens, Pseudomonas fluorescens PTA-3749, Pseudomonas sp. formerly Pseudomonas fluorescens 55084, Pseudomonas fluorescens CGA 266446 55171, Pseudomonas fluorescens biovar I: MB101 PTA-7841, Pseudomonas fluorescens 53860, Pseudomonas putida Trevisan Migula ATCC 12633, Strain Designations: [A.3.12, ATCC 23467, NCIB 9494, NCTC 10936, R.Y. Stanier 90], Pseudomonas aeruginosa Schroeter Migula ATCC 9721 Strain Designations: NRS 112 [NRRL B-7, R. Hugh 814], Pseudomonas protegens Ramette et al. ATCC BAA-477 Strain Designations: Pf-5, Pseudomonas brenneri ATCC 49642 Strain Designations: P17, Pseudomonas protegens Ramette et al. ATCC 17386 Strain Designations: 1 [IFO 15842], Pseudomonas chlororaphis Guignard and Sauvageau Bergey et al. ATCC 17809 Strain Designations: 388 [NRRL B-1854], Pseudomonas putida Trevisan Migula ATCC 31483 Strain Designations: 3P, Pseudomonas putida Trevisan Migula ATCC 11250 Strain Designations: Tr. 23 [NCIB 10098], Pseudomonas aeruginosa Schroeter Migula ATCC 31156 Strain Designations: KY 3955 [FERM-P 2611], Pseudomonas chlororaphis Guignard and Sauvageau Bergey et al. ATCC 17812 Strain Designations: 391 [NRRL B-1869], pSCH5102 ATCC 87426, Pseudomonas chlororaphis Guignard and Sauvageau Bergey et al. ATCC 17813 Strain Designations: 392 [Lysenko 4, NRRL B-2075], Burkholderia cepacia Palleroni and Holmes Yabuuchi et al. ATCC 29424 (Pseudomonas fluorescens Migula) Strain Designations: PHK, Pseudomonas putida Trevisan Migula ATCC 12842 Strain Designations: NCIB 8865 strain C01, Pseudomonas aeruginosa Schroeter Migula ATCC 12121 Strain Designations: P-6, Pseudomonas aeruginosa Schroeter Migula ATCC 9721 Strain Designations: NRS 112 [NRRL B-7, R. Hugh 814], Pseudomonas chlororaphis Guignard and Sauvageau Bergey et al. ATCC 17810 Strain Designations: 389 [NRRL B-1854], Pseudomonas migulae ATCC 39005 Strain Designations: PMW6 [NCIB 11615], Pseudomonas aeruginosa Schroeter Migula ATCC 8689 Strain Designations: 668 [R. Hugh 810, USDA 604], Pseudomonas aeruginosa Schroeter Migula ATCC 10796 Strain Designations: 68, Pseudomonas putida Trevisan Migula ATCC 11172 Strain Designations: NCIB 8251 [DSM 6521], Pseudomonas aeruginosa Schroeter Migula ATCC 142 Strain Designations: AMC [NRRL B-12], Pseudomonas sp. ATCC 49323 Strain Designations: NCIB 10586, Pseudomonas chlororaphis Guignard and Sauvageau Bergey et al. ATCC 17814 Strain Designations: 393 [NRRL B-1632; 261], Pseudomonas chlororaphis Guignard and Sauvageau Bergey et al. ATCC 17411 Strain Designations: 32 [NCIB 9402, NRRL B-977; NCTC 7357], Pseudomonas chlororaphis Guignard and Sauvageau Bergey et al. ATCC 17419 Strain Designations: 41, Pseudomonas fluorescens Migula ATCC Strain Designations: 197 [PJ 290], Pseudomonas fluorescens Migula ATCC 17397 Strain Designations: 12 [ATCC 25323, NIH 11, den Dooren de Jong 216], Pseudomonas chlororaphis Guignard and Sauvageau Bergey et al. ATCC 17415 Strain Designations: 36, Pseudomonas fluorescens Migula ATCC 17816 Strain Designations: 401 [C71A, IFO 15831, PJ 187], Pseudomonas fluorescens Migula ATCC 17638 Strain Designations: 271 [B-1612], Pseudomonas fluorescens Migula ATCC 31086 Strain Designations: D946-B83 [BU 2183, FERM-P 3328], Pseudomonas fluorescens Migula ATCC 49036 Strain Designations: A1 [IFO 15839], Pseudomonas chlororaphis Guignard and Sauvageau Bergey et al. ATCC 17811 Strain Designations: 390 [NRRL B-1095], Pseudomonas aeruginosa Schroeter Migula ATCC 8707 Strain Designations: 604 [R. Hugh 811], Pseudomonas fluorescens Migula ATCC 17583 Strain Designations: 216 [PJ 885], Pseudomonas chlororaphis Guignard and Sauvageau Bergey et al. ATCC 17461 Strain Designations: 86, Pseudomonas fluorescens Migula ATCC 49271 Strain Designations: F 1848 [CDC 93], Pseudomonas chlororaphis Guignard and Sauvageau Bergey et al. ATCC 17414 Strain Designations: 35 [NRRL B-1541], Pseudomonas fluorescens Migula ATCC 17926 Strain Designations: NRRL B-3178 [4, IFO 15841], Pseudomonas fluorescens Migula ATCC 33231 Strain Designations: B69, Pseudomonas mosselii Dabboussi et al. ATCC 49838 Strain Designations: AmMS 257, Pseudomonas fluorescens Migula ATCC 21637 Strain Designations: [IFO 3081], Pseudomonas fluorescens Migula ATCC 31948 Strain Designations: A506 [A5-06], Pseudomonas protegens Ramette et al. ATCC BAA-477D-5 Strain Designations: Genomic DNA from Pseudomonas protegens strain Pf-5 [ATCC BAA-477], ATCC 21539, ATCC 31951, ATCC 55034, Pseudomonas synxantha Ehrenberg Holland ATCC 9890 Strain Designations: [CCEB 293, NRRL B-780], Pseudomonas chlororaphis Guignard and Sauvageau Bergey et al. ATCC 17413 Strain Designations: 34 [NRRL B-1098], Aspergillus niger van Tieghem ATCC 16888, Penicillium roqueforti Thom ATCC 10110, Herminiimonas sp. ATCC 49643 Strain Designations: NOX, Rhizopus stolonifer Ehrenberg: Fries Lind ATCC 14037, or MA1-6 ATCC CRL-1783.

In some embodiments, the one or more microbial organisms of the microorganism consortium may include, but are not limited to, any strain of Pseudomonas calcis.

In some embodiments, the one or more microbial organisms of the microorganism consortium may include, but are not limited to, Acrothcium, Actinomyces, Alternaria, Arthrobotrys, Aspergillus, Candida, Cephalosporium, Cladosporium, Curvularia, Cunninghamella, Chaetomium, Cryptococcus, Debaryomyces, Fusarium, Glomus, Helminthosporium, Hansenula, Klockera, Micromonospora, Mortierella, Myrothecium, Oidiodendron, Paecilomyces, Penicillium, Phoma, Pichia, Populospora, Rhizoctonia, Rhizopus, Rhodotorula, Saccharomyces, Schizosaccharomyces, Schwanniomyces, Sclerotium, Torula, Trichoderma, or Yarrowia.

In some embodiments, the one or more microbial organisms of the microorganism consortium may include, but are not limited to, Mortierella alliacea, Mortierella alpina, M polycephala, Mortierella elongata, Mortierella spinosa, Mortierella gamsii, Mortierella isabellina, Mortierella humilis, or Mortierella reticulata.

In some embodiments, the one or more microbial organisms of the microorganism consortium may include, but are not limited to, Aspergillus rugulosus MB 277104, Aspergillus rugulosa ATCC 16820, Aspergillus rugulosa ATCC 32659, Aspergillus rugulosa ATCC 32623, Aspergillus rugulosa ATCC 32660, Aspergillus rugulosa ATCC 32661, Aspergillus rugulosa ATCC 58398, Aspergillus rugulosa ATCC 64625, Aspergillus rugulosa ATCC 16381, Aspergillus rugulosa ATCC 64624, Aspergillus rugulosa ATCC 22283, Aspergillus niger, and Aspergillus oryzae.

In some embodiments, the one or more microbial organisms of the microorganism consortium may include, but are not limited to, Penicillium bilaiae ATCC 18309, Penicillium bilaiae ATCC 20851, Penicillium bilaiae ATCC 22348, Penicillium bilaiae NRRL 50162, Penicillium bilaiae NRRL 50169, Penicillium bilaiae NRRL 50776, Penicillium bilaiae NRRL 50777, Penicillium bilaiae NRRL 50778, Penicillium bilaiae NRRL 50777, Penicillium bilaiae NRRL 50778, Penicillium bilaiae NRRL 50779, Penicillium bilaiae NRRL 50780, Penicillium bilaiae NRRL 50781, Penicillium bilaiae NRRL 50782, Penicillium bilaiae NRRL 50783, Penicillium bilaiae NRRL 50784, Penicillium bilaiae NRRL 50785, Penicillium bilaiae NRRL 50786, Penicillium bilaiae NRRL 50787, Penicillium bilaiae NRRL 50788, Penicillium bilaiae RS7B-SD1, Penicillium brevicompactum AgRF 18, Penicillium canescens ATCC 10419, Penicillium expansum ATCC 24692, Penicillium expansum YT02, Penicillium fellatanum ATCC 48694, Penicillium gaestrivorus NRRL 50170, Penicillium glabrum DAOM 239074, Penicillium glabrum CBS 229.28, Penicillium janthinellum ATCC 10455, Penicillium lanosocoeruleum ATCC 48919, Penicillium radicum ATCC 201836, Penicillium radicum FRR 4717, Penicillium radicum FRR 4719, Penicillium radicum N93/47267 and/or Penicillium raistrickii ATCC 10490, or Penicillium purpurogenum var. Rubrisclerotium MB 124011.

In some embodiments, the one or more microbial organisms of the microorganism consortium may include, but are not limited to, a yeast microorganism. Yeast microorganisms include genera and species within the Ascomycota phylum, including true yeasts and fission yeasts. Preferred yeast microorganisms may include Saccharomyces genus and combinations thereof. Examples of useful yeast include for example Saccharomyces cerevisiae. In one embodiment, a microorganism consortium in culture medium contains a Saccharomyces cerevisiae. In a more specific embodiment, the subject yeast is Saccharomyces cerevisiae CNCM 1-3060, Saccharomyces cerevisiae NCYC R397, Saccharomyces cerevisiae CNCM 1-3399, Saccharomyces cerevisiae NCYC R646, Saccharomyces cerevisiae CBS 493.9, Saccharomyces cerevisiae CNCM 1-1077, Saccharomyces cerevisiae NCYC Sc 47, Saccharomyces cerevisiae CNCM 1-4407, Saccharomyces cerevisiae MUCL 39885, Saccharomyces cerevisiae NCYC R404, Saccharomyces cerevisiae NCYC R404, Saccharomyces cerevisiae PCM KKP 2059p, or Saccharomyces cerevisiae CNCM 1-1079.

Additionally, the at least one yeast may be selected from a group comprising Acrothcium robustum ATCC 10715, Acrothecium capsici ATCC 10714, Candida montana, Candida etchellsii ATCC 60167, Candida versatilis ATCC 60121 or Candida robusta Diddens et Lodder.

In one embodiment, the yeast is a species of Saccharomyces. In a more specific embodiment, the yeast is Saccharomyces cerevisiae. In a still more specific embodiment, the yeast is a specific strain of Saccharomyces cerevisiae that is generally recognized as safe for ingestion or approved for use in animal feed or human food, such as e.g., Saccharomyces cerevisiae NCYC Sc47, Saccharomyces cerevisiae PCM KKP 2059p, or Saccharomyces cerevisiae IFO 0203.

### II. Methods of Use

The compositions disclosed herein are useful in agriculture applications, environmental remediation, and in some embodiments as chemical replacements. The present disclosure encompasses methods of benefiting a plant, a plant product such as cut grain or plant material, or plant growing medium by applying the microbial-based composition to the target plant, plant product, or plant growing medium.

The term “plant growing medium” is to be understood as encompassing any medium known to facilitate the growth of a plant, including but not limited to soil, hydroponic solution, a culture plate, air treated with micronutrients, macronutrients, and water, or any other medium known to facilitate plant growth. The term “soil” as used herein should be understood to encompass all plant growth mediums, such as hydroponic solution, unless otherwise indicated.

The methods may be used to partially or completely replace chemical compositions, or to enhance the activity of one or more chemical compositions. The methods may be used to benefit an environment, such as enhancing soil for agriculture purposes and reducing odor associated with waste.

The microbial-based compositions disclosed herein are useful in agriculture applications, including but not limited to soil enrichment, plant enrichment, and enhancing biodegradation. Methods of utilizing the microbial-based composition in agricultural methods are also provided.

Methods of utilizing the microbial-based composition in soil enrichment include applying the microbial-based composition to the soil to be enriched. The microbial-based composition may be in liquid or dry form and applied to the soil by methods known in the art. Exemplary methods include spraying, dropping, scattering, and dusting the target soil. Also, the microbial-based composition may be applied to a water source that feeds the target soil.

In another embodiment, the microbial-based composition may be used for plant enrichment. Methods of plant enrichment include applying the microbial-based compositions of the disclosure to the soil or water source of the plant as described herein. Also, the microbial-based composition may be added to the water of cut flowers or plants. In another embodiment, seeds may be soaked in a microbial-based composition of the disclosure prior to planting. It will be recognized that it may be beneficial to combine any of the methods described herein for soil and plant enrichment.

The one or more products made by the processes disclosed and described herein can be applied using any conventional system for applying liquid or solid to a seed or foliar surface or locus. Most commonly, application by spraying will be found most convenient, but other techniques, including application by tumbling, brush or by rope-wick can be used if desired. For spraying, any conventional atomization method can be used to generate spray droplets, including hydraulic nozzles and rotating disk atomizers. Introduction of the composition into an irrigation system can be used.

For folliar surface or locus applications, the application rate of the composition can be (in grams per square centimeter of plant or leaf surface or millileters per gram of plant or leaf weight) between about 0.01 g/cm² to about 10.0 g/cm², between about 0.01 ml/g to about 10.0 ml/g dry weight, between about 0.2 g/cm² to about 2.0 g/cm², between about 0.2 ml/g to about 2.0 ml/g, between 0.3 g/cm² to about 1.5 g/cm², between 0.3 ml/g to about 1.5 ml/g, between about 0.4 g/cm² to about 1.0 g/cm², between about 0.4 g/ml to about 1.0 g/ml, greater than about 0.4 g/cm² to about 1.0 g/cm² or or greater than between about 0.4 g/ml to about 1.0 g/ml, applied in the soil or as a foliar application to the foliage or the locus of the plant.

The one or more products made by the processes disclosed herein can be applied to a single plant (e.g., a houseplant or garden ornamental) or to an assemblage of plants occupying an area. In some embodiments, the product is applied to an agricultural or horticultural crop, more especially a food crop. A “food crop” herein means a crop grown primarily for human or animal consumption. Methods of use are appropriate both for field use and in protected cultivation, for example, greenhouse use.

One or more products made by the processes dislosed herein may be applied to a cut plant product, meaning any part of any plant that has been cut off from the plant.

The one or more products made by the processes disclosed herein may also be beneficial for gramineous (grass family) crops such as cereal crops, including corn, wheat, barley, oats and rice. The one or more products made by the processes disclosed herein may also be appropriate for non-gramineous crops, including vegetable crops, fruit crops, broad-leaved field crops such as soybeans, seed crops or a crop of any species grown specially to produce seed. The terms “fruit” and “vegetable” herein are used in their agricultural or culinary sense, not in a strict botanical sense; for example, tomatoes, cucumbers and zucchini are considered vegetables for present purposes, although botanically speaking it is the fruit of these crops that is consumed.

Vegetable crops for which the one or more products made by the processes disclosed herein can be found useful include without limitation: leafy and salad vegetables such as amaranth, beet greens, bitterleaf, bok choy, Brussels sprout, cabbage, catsear, celtuce, choukwee, Ceylon spinach, chicory, Chinese mallow, chrysanthemum leaf, corn salad, cress, dandelion, endive, epazote, fat hen, fiddlehead, fluted pumpkin, golden samphire, Good King Henry, ice plant, jambu, kai-lan, kale, komatsuna, kuka, Lagos bologi, land cress, lettuce, lizard's tail, melokhia, mizuna greens, mustard, Chinese cabbage, New Zealand spinach, orache, pea leaf, polk, radicchio, rocket (arugula), samphire, sea beet, seakale, Sierra Leone bologi, soko, sorrel, spinach, summer purslane, Swiss chard, tatsoi, turnip greens, watercress, water spinach, winter purslane and you choy, flowering and fruiting vegetables such as acorn squash, Armenian cucumber, avocado, bell pepper, bitter melon, butternut squash, caigua, Cape gooseberry, cayenne pepper, chayote, chili pepper, cucumber, eggplant (aubergine), globe artichoke, luffa, Malabar gourd, parwal, pattypan squash, perennial cucumber, pumpkin, snake gourd, squash (marrow), sweetcorn, sweet pepper, tinda, tomato, tomatillo, winter melon, West Indian gherkin and zucchini (courgette), podded vegetables (legumes) such as American groundnut, azuki bean, black bean, black-eyed pea, chickpea (garbanzo bean), drumstick, dolichos bean, fava bean (broad bean), French bean, guar, haricot bean, hemp, horse gram, Indian pea, kidney bean, lentil, lima bean, marijuana, moth bean, mung bean, navy bean, okra, pea, peanut (groundnut), pigeon pea, pinto bean, rice bean, runner bean, soybean, tarwi, tepary bean, urad bean, velvet bean, winged bean and yardlong bean, bulb and stem vegetables such as asparagus, cardoon, celeriac, celery, elephant garlic, fennel, garlic, kohlrabi, kurrat, leek, lotus root, nopal, onion, Prussian asparagus, shallot, Welsh onion and wild leek, root and tuber vegetables, such as ahipa, arracacha, bamboo shoot, beetroot, black cumin, burdock, broadleaf arrowhead, camas, canna, carrot, cassava, Chinese artichoke, daikon, earthnut pea, elephant-foot yam, ensete, ginger, gobo, Hamburg parsley, horseradish, Jerusalem artichoke, jicama, parsnip, pignut, plectranthus, potato, prairie turnip, radish, rutabaga (swede), salsify, scorzonera, skirret, sweet potato, taro, ti, tigernut, turnip, ulluco, wasabi, water chestnut, yacon and yam, and herbs, such as angelica, anise, basil, bergamot, caraway, cardamom, chamomile, chives, cilantro, coriander, dill, fennel, ginseng, jasmine, lavender, lemon balm, lemon basil, lemongrass, marjoram, mint, oregano, parsley, poppy, saffron, sage, star anise, tarragon, thyme, turmeric and vanilla.

Fruit crops for which the present disclosure can be found useful include without limitation apple, apricot, banana, blackberry, blackcurrant, blueberry, boysenberry, cantaloupe, cherry, citron, clementine, cranberry, damson, dragonfruit, fig, grape, grapefruit, greengage, gooseberry, guava, honeydew, jackfruit, key lime, kiwifruit, kumquat, lemon, lime, loganberry, longan, loquat, mandarin, mango, mangosteen, melon, muskmelon, orange, papaya, peach, pear, persimmon, pineapple, plantain, plum, pomelo, prickly pear, quince, raspberry, redcurrant, starfruit, strawberry, tangelo, tangerine, tayberry, ugli fruit and watermelon.

Seed crops, for example, may comprise any specialized crops used to produce seed of any plant species. The microorganism consortium may be found useful in applications direct to, including but not limited to cereals (e.g., barley, corn (maize), millet, oats, rice, rye, sorghum (milo) and wheat), non-gramineous seed crops such as buckwheat, cotton, flaxseed (linseed), mustard, poppy, rapeseed (including canola), safflower, sesame and sunflower.

Silage crops may comprise, but are not limited to, corn, grain sorghum, Bermuda grass, star grass, limpograss, forage sorghum, pearl millet, ryegrass, alfalfa, red clover, hairy indigo, alyce clover, shamrock, vicia sativa, aeschynomene, and rhizoma perennial peanut. The present microbial-based composition may be used in conjunction with or applied to hay or oats to make haylage and oatlage, respectively.

Other crops, not fitting any of the above categories, for which the present disclosure can be found useful include without limitation sugar beet, sugar cane, hops, cannibis (such as cannabis sativa, cannabis indica, and cannabis ruderalis or hemp or marijuana), and tobacco.

Moreover, the products made by the processes disclosed herein may be useful in any hydroponic growing operation, on any growing plant, or on any plant rooted in soil.

Each of the crops listed above has its own particular nutrition and disease protection needs. Further optimization of compositions described herein for particular crops can readily be undertaken by those of skill in the art, based on the present disclosure, without undue experimentation.

In another embodiment, the addition of the microbial-based composition to one or more wastes may have the effect of enhancing biodegradation of the various wastes. Such wastes include, without limitation, wastewater, runoff, food waste, waste produced by humans or animals, and landfill waste. The microbial-based composition also has the effect of enhancing composting.

The microbial-based composition may be provided either dried or in liquid form to a waste product. The microbial-based composition may be provided in a variety of amounts with respect to the weight of the waste product depending on the waste product. In some embodiments, the microbial-based composition is provided in an amount ranging from about 0.5 to 95.5 wt % of the total weight of the waste product. In some embodiments, the microbial-based composition is provided in an amount ranging from about 0.5 to 75 wt % of the total weight of the waste product. In some embodiments, the microbial-based composition is provided in an amount ranging from about 0.5 to 50 wt % of the total weight of the waste product. In some embodiments, the microbial-based composition is provided in an amount ranging from about 0.5 to 25 wt % of the total weight of the waste product. In another embodiment, the microbial-based composition is provided in an amount ranging from about 1 to about 3 wt % of the total weight of the waste product. In another embodiment, the amount of microbial-based composition provided to the waste is about 2 wt % of the total amount of waste.

The microbial-based composition may be provided in either dry form, liquid form or through the spray. Methods of treating waste products include without limitation, spraying, dusting, sprinkling, liquid inoculation, misting, fumigating, aerosolizing, pouring or pumping the microbial-based composition into the wastewater or other waste product, and other methods known in the art.

It should be understood that, in treating wastewater, waste product, or in another usage described herein, the microbial-based composition compositition used may be provided in the form of pure concentrate (100% concentration) or a diluted composition with additional excipients in the dosage form (i.e. the amount of active ingredient in the composition is less than or equal to 99.99%, and the remainder consists of inactive excipients). If diluted, the amount of microbial-based composition compositition dispensed in the various dosage forms may range from about 1 to 30%, more preferably between about 4 to 8%. One of skill in the art will appreciate that the volume of active component added to the composition will need to be adjusted to account for the dilution and to ensure the end composition comprises the appropriate final concentration of microbial-based composition compositition. One of skill in the art will also appreciate that the various components of the microbial-based composition compositition may be provided in a variety of dosage forms including, but not limited to liquid solution or suspension, emulsion, aerosol, slow release matrices, and the like.

Typical concentration range of microorganisms administered is 1E+3 to 1E+13 cells per day. Preferably, at least about 1E+6, at least about 1E+7, at least about 1E+8 cells per day are administered. However, it will be appreciated that the number of microorganisms to be administered will vary according to a number of parameters including the target's size and need.

The concentration of microbial-based composition compositition which is used for environmental, systemic, topical, or foliar application will vary widely depending upon the nature of the particular formulation, means of application, environmental conditions, and degree of activity. Typically, the microbial-based composition compositition will be present in the applied formulation at a concentration of at least about 1% by weight and may be up to and including about 99% by weight. Dry formulations of the microbial-based composition compositition may be from about 1% to about 99% or more by weight of the composition, while liquid formulations may generally comprise from about 1% to about 99% or more of the composition by weight. As such, a variety of formulations are preparable, including those formulations that comprise from about 5% to about 95% or more by weight of the microbial-based composition mix, including those formulations that comprise from about 10% to about 90% or more by weight of the microbial-based composition compositition. Naturally, formulations may comprise from about 15% to about 85% or more by weight of the composition, and formulations comprising from about 20% to about 80% or more by weight of the microbial-based composition compositition are also considered to fall within the scope of the microbial-based composition.

In compositions in which intact microorganisms are included, preparations will generally contain from about 1E+4 to about 1E+8 cells/mg, although in certain embodiments it may be desirable to utilize formulations comprising from about 1E+2 to about 1E+4 cells/mg, or when more concentrated formulations are desired, compositions comprising from about 1E+8 to about 1E+10 or 1E+11 cells/mg may also be formulated.

A typical field application rate per hectare may range on the order of from about 50 g/hectare to about 500 g/hectare of composition, or alternatively, from about 500 g/hectare to about 1000 g/hectare may be utilized. In certain instances, it may even be desirable to apply the composition to a target area at an application rate of from about 1000 g/hectare to about 5000 g/hectare or more of composition. In fact, all application rates in the range of from about 50 g of composition per hectare to about 10,000 g/hectare are contemplated to be useful in the management, control, and killing of target insect pests using such insecticidal formulations. As such, rates of about 100 g/hectare, about 200 g/hectare, about 300 g/hectare, about 400 g/hectare, about 500 g/hectare, about 600 g/hectare, about 700 g/hectare, about 800 g/hectare, about 900 g/hectare, about 1 kg/hectare, about 1.1 kg/hectare, about 1.2 kg/hectare, about 1.3 kg/hectare, about 1.4 kg/hectare, about 1.5 kg/hectare, about 1.6 kg/hectare, about 1.7 kg/hectare, about 1.8 kg/hectare, about 1.9 kg/hectare, about 2.0 kg/hectare, about 2.5 kg/hectare, about 3.0 kg/hectare, about 3.5 kg/hectare, about 4.0 kg/hectare, about 4.5 kg/hectare, about 6.0 kg/hectare, about 7.0 kg/hectare, about 8.0 kg/hectare, about 8.5 kg/hectare, about 9.0 kg/hectare, and even up to and including about 10.0 kg/hectare or greater of composition may be utilized in certain agricultural, industrial, and domestic applications of the insecticidal formulations described herein.

In some embodiments, a method for preserving silage quality and reducing damage to a cut plant product from at least one plant pathogen may comprise, at least, administering a therapeutic amount of the microbial-based composition, or a “finished product,” to the at least one cut plant product, wherein the finished product comprises A) a microorganism consortium comprising at least five microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma vixens, wherein the microorganisms are co-cultured; B) at least one carbon source selected from the group consisting of rum, molasses, glucose, starch, cellulose, fructose, and sucrose; and C) dechlorinated water.

In some embodiments, the cut plant product may comprise silage, oatlage, or haylage. In some embodiments, the silage may comprise at least one selection from the group comprising corn, grain sorghum, Bermuda grass, star grass, limpograss, forage sorghum, pearl millet, ryegrass, alfalfa, red clover, hairy indigo, alyce clover, shamrock, vicia sativa, aeschynomene, and rhizoma perennial peanut.

The method of contacting the silage with the microbial-based composition may transpire at any step in the silage process, including but not limited to the respiration, fermentation, or feedlot management stages. The stages of silage may be alternatively be described as cellular respiration, lactic acid and acetic acid production, lactic acid formation, lactic acid fermentation, and aerobic decomposition. The stages of silage may also be described as comprising an aerobic phase, a lag phase, a fermentation phase, and a stable phase. The present microbial-based composition is effective at each stage and has the effects, at least, of reducing respiration and preserving the benefits of fermentation during the feedlot management stage.

Indeed, in some embodiments, it is contemplated that the microbial-based composition may be applied at more than one stage of the silage process, and in some embodiments more than once within or during a single stage.

Methods of contacting the cut plant products with the microbial-based composition include any of those mentioned herein, including but not limited to spraying, dusting, sprinkling, liquid inoculation, misting, fumigating, aerosolizing, pouring or pumping the microbial-based composition onto the cut plant product, or soaking the cut plant product in the present microbial-based composition.

### III. Experimental Results

The microbial-based composition described above can be used for replacing and/or enhancing chemical and organic fertilizers, surfactants as well as biojuvants in conjunction with one or more auxiliary agents for agricultural spray applications.

### Experiment 1

The microbial-based composition was tested in an ensilage operation to determine whether the microbial-based composition improves the drying time and overall quality of silage. The microbial-based composition was also tested in a soil conditioning study.

First, the microbial-based composition was tested in ensilage processing of cut grain forage. It can be applied at the mower-conditioner (“haybine”), crimper, swather baler, chopper or blower. In the present experiment, the microbial-based composition was applied at 0.09-0.15% (w/w) of fresh cut forage.

To test its properties, the microbial-based composition was sprayed onto fresh cut forage at 0.10% (w/w), replacing a non-ionic surfactant containing alcohol ethoxylates, propylene glycol, dimethlypolysiloxane. Only negative control was implemented in the trial. The microbial-based composition's drying properties were examined.

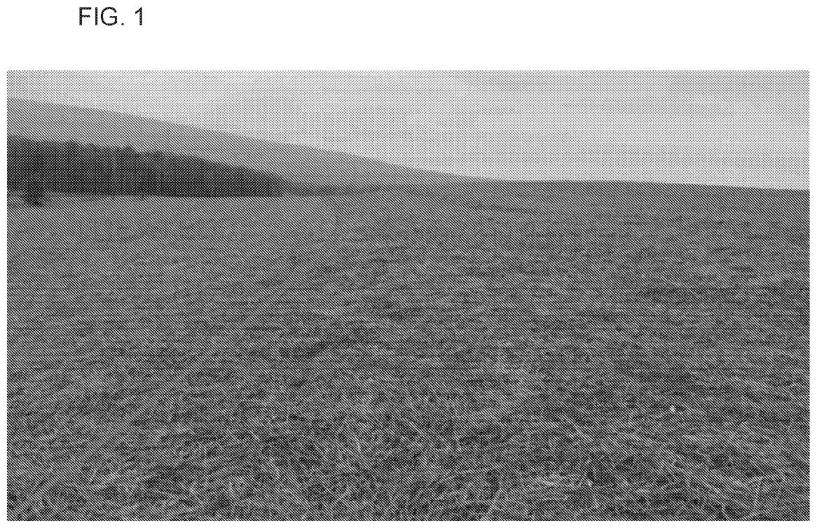

FIG. 1 indicates the effectiveness of the composition at drying the fresh cut grain forage. The treated section dried within 1 to 2 days, while the untreated section remained wet. Surprisingly, the microbial-based composition's drying time was found to be similar to the conventional chemical treatment. Moreover, the microbial-based composition also showed better relative feed value (RFV), higher lactic acid producing bacteria, improved nutrient retention, reduction in storage losses and processing time, and better palatability.

Second, the microbial-based composition's effect on soil was also tested. The study site included twelve 3×8 meter plots with 4 treatments, including a negative control. Treatment 1 comprised 144 ml of the microbial-based composition (FIG. 3) plus 2.256 liters of water. Treatment 2 comprised 216 ml of the microbial-based composition (FIG. 3) plus 2.184 liters of water. Treatment 3 comprised 288 ml of the microbial-based composition (FIG. 3) plus 2.212 liters of water. Treatment 4 comprised 2.400 liters of water.

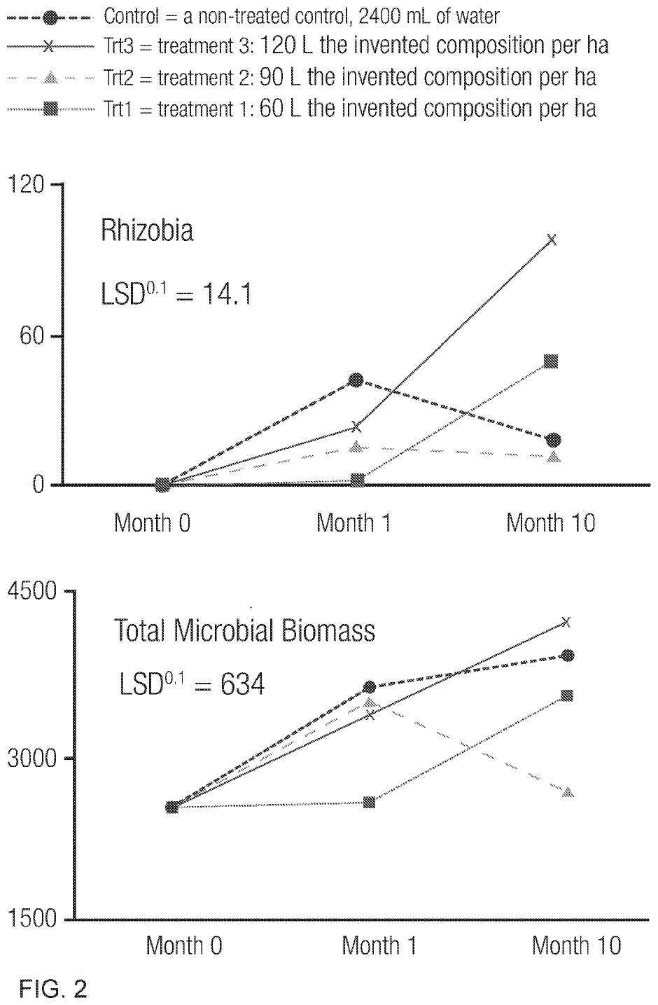

FIG. 2 illustrates the impact of the microbial-based composition on both rhizobia and total microbial biomass. Rhizobia are soil bacteria that fix nitrogen after becoming established inside the root nodules of legumes. Nitrogen inputs through fertilizers causes harsh ecological concerns, making the presence of rhizobia in soil a vital objective. Total microbial biomass (bacteria and fungi) is the measurement of the mass of living components in soil organic matter. Total microbial biomass in the decomposed plant, animal residue, and soil organic matter released lower levels of carbon dioxide and enhance plant-available nutrients.

Rhizobia levels in the soil bacteria in Treatment 3 (Trt3) showed a significant increase during the study after the first split application on (Trt3: 98.05 ng g-1 and Control: 18.72 ng g-1) (FIG. 2). Protozoa were higher in Trt3 (81 ng g-1) than control (36.61 ng g-1). Significant differences were observed in soil enzymatic activity, as alpha-glucosidase (5.19 μg) and FDA hydrolase (8270m) activities were higher in Trt3 than the control (respectively 4.63 μg and 5137 μg). Soil enzymes increase the reaction rate at which plant residues decompose and release plant available nutrients. Enzymes such as hydrolase and glucosidase facilitate the breakdown of organic matter.

Total microbial biomass had a strong positive correlation with total fungi, saprophytic fungi, rhizobia and protozoa biomass in Trt3. Researchers also observed a 20% increase in the ration of fungi to bacteria over a ten-month period. Because Trt3 contained the highest amount of the microbial-based composition in the mixture, these results suggest a direct correlation between the microbial-based composition and the increase of soil quality.

### Experiment 2

In the second experiment, the microbial-based composition's effect on soil improvement, enhancement of plant root growth, yield improvement, and reduction in application of chemical fertilizers were studied. The microbial-based composition was applied to brussell sprouts. The brussell sprout seeds were allowed to germinate for 21 days.

The seeds were soaked for 24 hours in 20 mL of chlorine-free water (Control) and in 20 mL of chlorine-free water with various dilutions of the microbial-based composition in a covered petri dish at ambient temperature. Six plastic containers were filled with 400 g each of moist, sterile coco coir. Fourteen seeds per container were inserted approximately 1 cm into the coir and covered gently.

The control seeds were given 150 ml deionized water. Treatment 1 comprised the microbial-based composition at a dilution factor of 1000 (0.1%) in 150 ml deionized water. Treatment 2 comprised the microbial-based composition at a dilution factor of 500 (0.2%) in 150 ml deionized water. Treatment 3 comprised the microbial-based composition at a dilution factor of 100 (1.0%) in 150 ml deionized water.

Upon emergence of seedlings, the containers were set in a south-facing window sill with a fan placed behind the containers. The fan alternately blew air for 30 minutes, then was off for 1 hour. The seedlings were monitored for water loss and were rotated daily.

Germination speed was recorded by measuring the time required for the seedling to germinate. Germination rate was recorded by measuring the number of seeds that germinated. Root growth was recorded by measuring the plant's root length and width in cm. Seedling total length was assessed by measuring the seedling's total length, total root length, and total mass.

Next, six seedlings were selected for each treatment (including control). Each seedling was placed in a fiber starter pot with 40 g seed-starter mix and treated with 100 ml of the appropriate treatment. Then, an indentation was made in the soil and a seedling was inserted, the indentation was closed, and approximately 20 g of additional soil, pre-treated with additional 50 mL of treatment solution. The excess moisture was allowed to drain into petri dishes that were set below the pots. The transplanted seedlings were placed on a windowsill 2 layers deep, the fan was replaced, and the seedlings were rotated both directions and layered daily to allow even growth.

From Monday to Friday, shoot height were measured from soil to meristem. Once true leaves began to develop, Miracle Gro All-Purpose Plant Food® fertilizer was applied Thursdays at the rate of 2.75 g in 4 L deionized water. The control was given 25 mL fertilizer and 25 mL water. All Treatments received 12.5 mL fertilizer and 37.5 mL deionized water. The seedlings were also each given 50 ml deionized water on Mondays.

The seedlings were then transplanted to a 10.3-gallon plastic can having one 0.24-inch hole drilled per side of the can two inches above the bottom of the can. The seedlings remained in these cans through maturity. LED grow bulbs were used 12 hours on, 12 hours off. One bulb was used per plant at full spectrum. For air flow, a box fan on a timer was used: 15 min on low followed by 30 min off, all day, every day.

The seedlings' microbiome was tested by collecting 10 g of soil, which was then ground into a fine power and sieved with a soil sieve. Next, 0.25 g of the sieved soil sample was weighed, and DNA was extracted from the soil using PowerSoil DNA Isolation Kit (Qiagen). A library and sequence were prepared with MinION (Oxford Nanopore Technology).

The plants received a weekly foliar spray per the dilution factors provided above. The plants received biweekly soil applications, which replaced that week's water application, at the rates of: Treatment 1: the microbial-based composition at 0.1% in 0.5 L of chlorine-free water. Treatment 2: the microbial-based composition at 0.2% in 0.5 L chlorine-free water, and Treatment 3: the microbial-based composition at 1.0% in 0.5 L chlorine-free water.

The plants also received a biweekly foliar spray (record volume) at the following rates: control: chlorine-free water only; Treatment 1: the microbial-based composition at 1% in chlorine-free water; Treatment 2: the microbial-based composition at 2% in chlorine-free water; and Treatment 3: the microbial-based composition at 4% in chlorine-free water. The water volumes were no more than necessary to mist the whole plant.

Plant height, leaf number, and leaf sizes were measured weekly. At harvest, approximately day 150, the following parameters were measured: a) soil testing; b) soil microbiome testing; c) total biomass (in grams); d) total root biomass (in grams); e) total number of sprouts; and f) total weight of sprouts (in grams).

External soil testing was accomplished by assessing: a) soil pH; b) available phosphorus (Mehlich-3); c) aqueous nitrate; d) soil-bound ammonium; e) cation content of Mg2+, Ca2+, K+, and Na+ in the soil; f) total nitrogen and phosphorous; and microbial presence and diversity via Qiagen PowerSoil DNA Extraction Kit and MinION (Oxford Nanopore Technology).

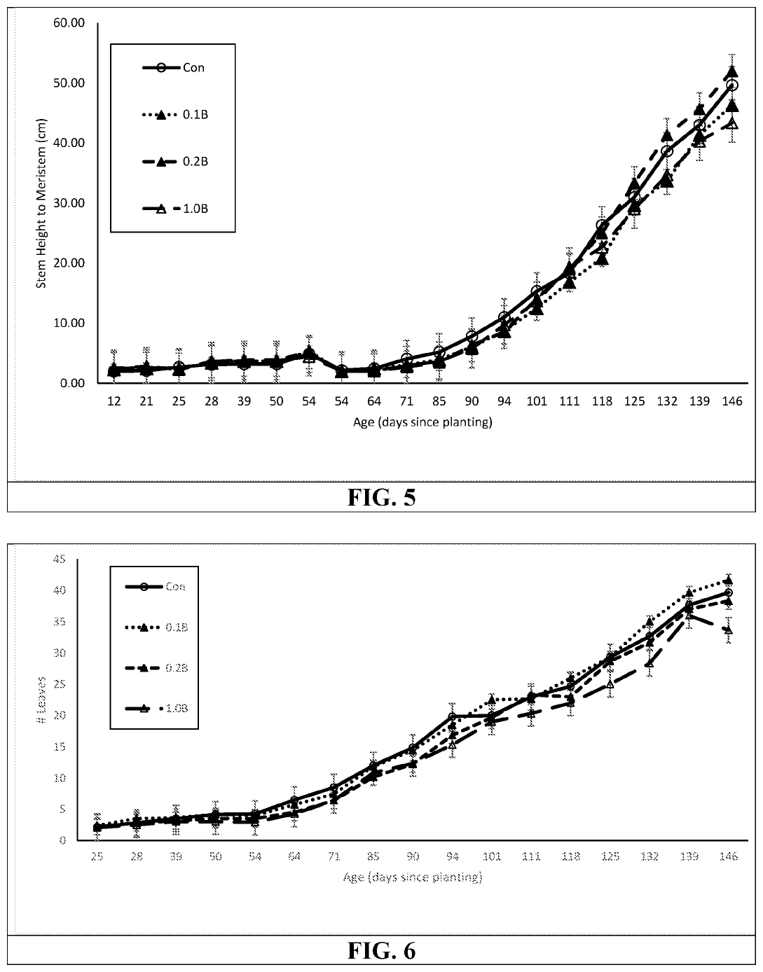

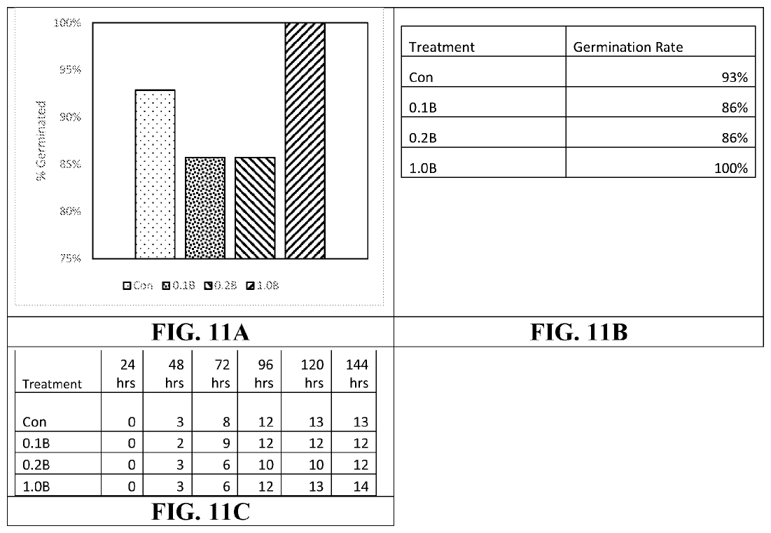

The results of Experiment 2 can be seen in FIG. 5 through FIG. 11C.

FIG. 5 illustrates shoot growth from seedling transplant until meristem removal prior to harvest. A decrease at day 54 from transplanting to final containers was shown, and the measurement was repeated to establish a new baseline. Error bars display standard deviation of each sample population. Confidence interval based upon final height and calculated per population: CI (95%)=49.67 cm+/−4.71 (0.1B), 6.73 (0.2B), 6.78 (1.0B).

FIG. 6 illustrates leaf production from seedling to maturity. Apical meristem leaves that were still forming were not counted. 0.1% microbial-based composition had the highest leaf count at 42 and is statistically significant from the control. 1.0% microbial-based composition had the lowest leaf count at 35 and is also statistically significant from the control. Error bars represent the standard deviation per population group. Confidence interval based upon final leaf count and calculated per population: CI (95%)=39.6 leaves+/−2.06(0.1B), 2.27(0.2B), 4.09 (1.0B), 4.41 (0.1P), 3.31 (0.2P).

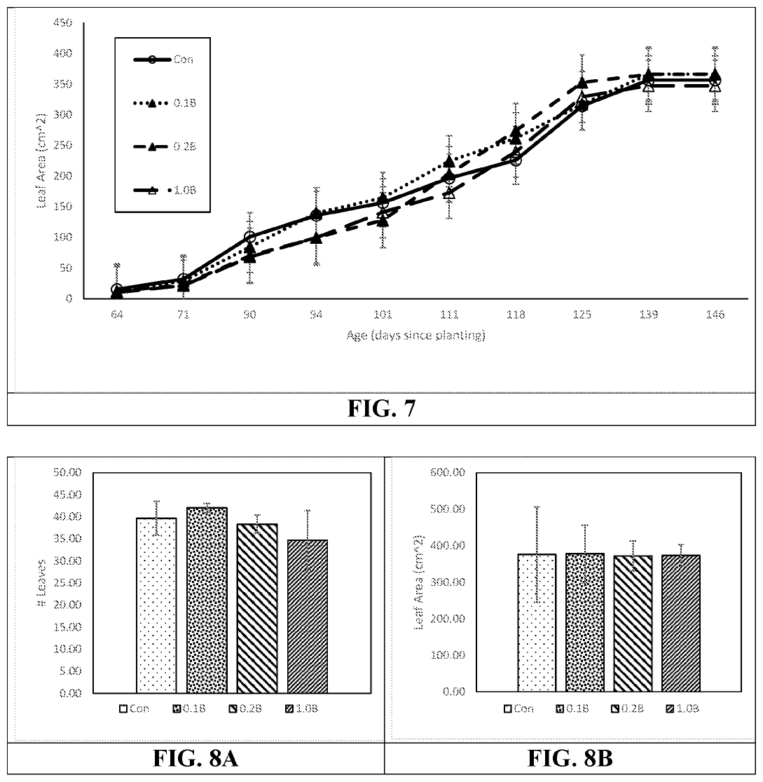

FIG. 7 illustrates leaf size growth rate as change in leaf length×length width from seedling to maturity. 0.1% B and 0.2% B showed consistently larger leaves from around 94 days, which was the date of Mid-Season Cull. Confidence interval based upon mature leaf size and calculated per population: CI (95%)=375.8 cu. cm+/−89.2 (0.1B), 71.6 (0.2B), 70.1 (1.0B).

FIG. 8A illustrates mature leaf number, wherein 0.1% microbial-based composition had the highest leaf count at 42 and is statistically significant from the control. 1.0% microbial-based composition had the lowest leaf count at 35 and is also statistically significant from the control. Error bars represent the standard deviation per population group. All other concentrations were not statistically different from the control. Confidence interval calculated per population: CI (95%)=39.6 leaves+/−2.06(0.1B), 2.27(0.2B), 4.09 (1.0B).

FIG. 8B illustrates mature leaf area as change in leaf length×length width from seedling to maturity. Error bars show standard deviation of each concentration population. 0.1% and 0.2% showed significantly larger leaves. Confidence interval calculated per population: CI (95%)=375.8 cu. cm+/−89.2 (0.1B), 71.6 (0.2B), 70.1 (1.0B).

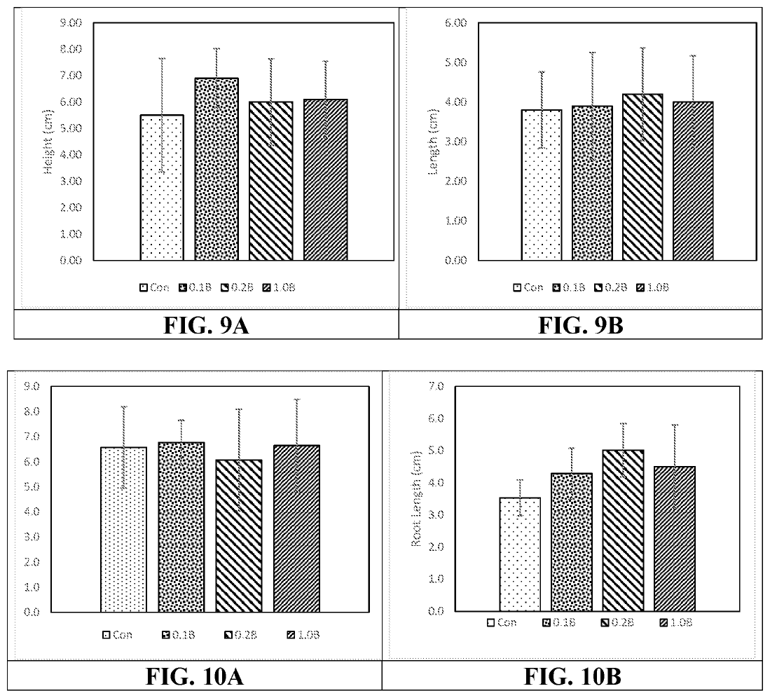

FIG. 9A illustrates seedling stem height (n=14). 0.1% microbial-based composition was significantly taller, on average, than control. 0.1% microbial-based composition produced significantly taller seedlings at 6.90 cm vs 5.5 cm of control. Confidence interval calculated per population: CI (95%)=5.5 cm+/−4.8 (0.1B), 4.7 (0.2B), 4.7 (1.0B).

FIG. 9B illustrates seedling root length (n=14). Confidence interval calculated per population: CI (95%)=3.8 cm+/−3.4 (0.1B), 3.3 (0.2B), 3.4 (1.0B).

FIG. 10A illustrates transplant stem height (n=6) upon first transplant. Wide variation observed within each population. Confidence interval calculated per population: CI (95%)=6.6 cm+/−1.0 (0.1B), 1.4 (0.2B), 1.3 (1.0B).

FIG. 10B illustrates transplant root length (n=6) upon first transplant. All treatments statistically longer than control with longest produced by 0.2% microbial-based composition. Confidence interval calculated per population: CI (95%)=3.5 cm+/−0.5 (0.1B), 0.5 (0.2B), 0.7 (1.0B).

Compositions useful in this disclosure include microorganisms and additives. The microorganisms may include species of bacteria and fungi, including yeast and mold species.

It is contemplated that where two or more microorganisms are present within one or more embodiments of the composition, the microorganisms may be co-cultured. The microorganisms may be propagated by methods known in the art. For example, the microorganisms may be propagated in a liquid medium under anaerobic or aerobic conditions. Suitable liquid mediums used for growing microorganisms include those known in the art. It is contemplated that, in some embodiments, the one or more strains of useful microorganisms described herein may be co-cultured under laboratory conditions in any combination before being transferred to one or more fermentation tanks. In some embodiments, the one or more strains of useful microorganisms may be co-cultured partly under laboratory conditions, then transferred to one or more fermentation tanks, where the co-culture process may continue. In other embodiments, the one or more strains of useful microorganisms may be co-cultured within one or more fermentation tanks.

In one embodiment, the microorganism consortium in culture media of the microbial-based composition includes live microorganisms. Preferably, at least one microorganism is included in the microorganism consortium in culture medium of the disclosure. More preferably, the compositions include consortia of two or more microorganisms. In another embodiment, the microorganism consortium in culture media of the microbial-based composition includes living and non-living microorganisms. In another embodiment, the microbial-based composition includes living or non-living microorganisms. Compositions containing non-living microorganisms may contain extracts of the microorganisms. Such extracts may be considered a liquid fermentation product of the living microorganisms. The extracts of microorganisms include, by way of example, enzymes, metabolites, proteins, and other substances that are produced by microorganisms and are capable of eliciting an effect on an environment regardless of the living status and/or metabolic states of the microorganism.

In one embodiment, the microbial-based composition may be fermented, and may therein to produce one or more fermentation products. The microbial-based composition may be fermented for about 1, 2, 3, 4, 5, 6, 7, 8, 9, 10, 11, 12, 13, 14, 15, 16, 17, 18, 19, 20, 21, 22, 23, 24, 25, or more days. Preferably, the microbial-based composition is fermented for at least about 15 to about 23 days. More preferably, the microbial-based composition is fermented for at least 21 days. More preferably, the microbial-based composition is fermented for at least 15 days.

In one embodiment, the process of fermentation may result in one or more fermentation products. The one or more fermentation products may comprise an organic acid, an alcohol, or another type of compound. In another embodiment, the one or more fermentation products may comprise fermented sugar cane molasses or fermented rice bran.

In one embodiment, the microbial-based composition contains a first mixture of live microorganisms in the at least one microorganism consortium in culture medium, wherein the live microorganisms are present at a collective “low titer”, and one or more additional non-pathogenic gram-positive bacteria of the Bacilli family that are capable of fermentation at a “high titer” for each additional individual species/strain of bacteria, and a fermenting yeast. By collective titer, what is meant is the individual titers of each of the first live microorganism adds up to the collective titer. To use an example of live lactic acid bacteria (LAB), if the (LAB) consists of 15,000 cfu/mL of a Bacillus sp., 12,000 cfu/mL of a Lactobacillus sp., and 10,000 cfu/mL of a Streptococcus sp., then the collective titer is 37,000 cfu/mL of LAB. In one embodiment, “low titer” is <300,000 cfu/mL, about 0.001-299,999 cfu/mL, about 1,000-250,000 cfu/mL, 5,000-200,000 cfu/mL, 10,000-150,000 cfu/mL, 15,000-100,000 cfu/mL, 20,000-75,000 cfu/mL, 25,000-60,000 cfu/mL, 30,000-55,000 cfu/mL, about 10,000 cfu/mL, about 11,000 cfu/mL, about 12,000 cfu/mL, about 13,000 cfu/mL, about 14,000 cfu/mL, about 15,000 cfu/mL, about 16,000 cfu/mL, about 17,000 cfu/mL, about 18,000 cfu/mL, about 19,000 cfu/mL, about 20,000 cfu/mL, about 21,000 cfu/mL, about 22,000 cfu/mL, about 23,000 cfu/mL, about 24,000 cfu/mL, about 25,000 cfu/mL, about 30,000 cfu/mL, about 35,000 cfu/mL, about 40,000 cfu/mL, about 45,000 cfu/mL, about 50,000 cfu/mL, about 55,000 cfu/mL, about 60,000 cfu/mL, about 65,000 cfu/mL, about 70,000 cfu/mL, about 75,000 cfu/mL, about 80,000 cfu/mL, about 85,000 cfu/mL, about 90,000 cfu/mL, about 95,000 cfu/mL, about 100,000 cfu/mL, about 105,000 cfu/mL, about 110,000 cfu/mL, about 115,000 cfu/mL, about 120,000 cfu/mL, about 125,000 cfu/mL, about 150,000 cfu/mL, about 175,000 cfu/mL, about 200,000 cfu/mL, about 225,000 cfu/mL, about 250,000 cfu/mL, about 275,000 cfu/mL, or about 299,000 cfu/mL.

In one embodiment, “high titer” is ≥300,000 cfu/mL, 300,000-10,000,000 cfu/mL, 500,000-1,000,000 cfu/mL, about 300,000 cfu/mL, about 325,000 cfu/mL, about 350,000 cfu/mL, about 375,000 cfu/mL, about 400,000 cfu/mL, about 425,000 cfu/mL, about 450,000 cfu/mL, about 475,000 cfu/mL, about 500,000 cfu/mL, about 525,000 cfu/mL, about 550,000 cfu/mL, about 575,000 cfu/mL, about 600,000 cfu/mL, about 625,000 cfu/mL, about 650,000 cfu/mL, about 675,000 cfu/mL, about 700,000 cfu/mL, about 725,000 cfu/mL, about 750,000 cfu/mL, about 775,000 cfu/mL, about 800,000 cfu/mL, about 825,000 cfu/mL, about 850,000 cfu/mL, about 875,000 cfu/mL, about 900,000 cfu/mL, about 925,000 cfu/mL, about 950,000 cfu/mL, about 975,000 cfu/mL, about 1,000,000 cfu/mL, about 1,250,000 cfu/mL, about 1,500,000 cfu/mL, about 1,750,000 cfu/mL, about 2,000,000 cfu/mL, about 2,500,000 cfu/mL, about 3,000,000 cfu/mL, about 3,500,000 cfu/mL, about 4,000,000 cfu/mL, about 4,500,000 cfu/mL, about 5,000,000 cfu/mL, about 5,500,000 cfu/mL, about 6,000,000 cfu/mL, about 6,500,000 cfu/mL, about 7,000,000 cfu/mL, about 7,500,000 cfu/mL, about 8,000,000 cfu/mL, about 8,500,000 cfu/mL, about 9,000,000 cfu/mL, about 9,500,000 cfu/mL, about 10,000,000 cfu/mL, about 1.5E+7, about 2E+7, about 2.5+7, about 3E+7, about 3.5E+7, about 4E+7, about 4.5E+7, about 5E+7, about 5.5E+7, about 6E+7, about 6.52E+7, about 7E+7, about 7.5E+7, about 8E+7, about 8.5E+7, about 9E+7, about 9.5E+7, about 1E+8, about 2E+8, about 3E+8, about 4E+8, about 5E+8, about 6E+8, about 7E+8, about 8E+8, about 9E+8, about 1E+9, about 3E+9, about 4E+9, about 5E+9, about 6E+9, about 7E+9, about 8E+9, about 9E+9, about 1E+10, about 2E+10, about 3E+10, about 4E+10, about 5E+10, about 6E+10, about 7E+10, about 8E+10, about 9E+10, or 1E+11 cfu/mL.

In those embodiments in which the microbial-based composition is in a dry form, the liquid microbial-based compositions described herein are dried. “Dry form” refers to a composition containing <15% water by weight, <14% water by weight, <13% water by weight, <12% water by weight, <11% water by weight, <10% water by weight, <9% water by weight, <8% water by weight, <7% water by weight, <6% water by weight, <5% water by weight, <4% water by weight, <3% water by weight, <2% water by weight, <1% water by weight, 1%-5% water by weight, 2%-6% water by weight, about 3% water by weight, about 4% water by weight, about 5% water by weight, about 6% water by weight, about 7% water by weight, or about 8% water by weight. In one embodiment, the dry form of the microbial-based composition is produced by evaporation, spray-drying, lyophilization, or the like. In some embodiments, the dry microbial-based composition is encapsulated or combined with an excipient to promote the stability and viability of the microbes over time and under varying temperature conditions. In some embodiments, the dry microbial-based composition is divided into micron scale particles that are subsequently coated with biocompatible polymers, such as polyethylene glycol (PEG), chitin, dextrin, polylactic glycolic acid copolymer (PLGA), polylactic acid (PLA), polyglycolic acid (PGA), or the like.

In one embodiment, the compositions may further comprise alcohol. Suitable alcohols include any known in the art including, without limitation, methanol, ethanol, n-propanol, allyl alcohol, n-propanol, isopropanol, sec-propanol, n-butanol, sec-butanol, isobutanol, t-butanol, and tert-amyl-alcohol. The weight fraction of the alcohol in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The compositions of the disclosure may be in liquid or dry form. The composition may comprise an aqueous suspension of components. This aqueous suspension may be provided as a concentrated stock solution which is diluted prior to application or as a diluted solution ready-to-use. Also, the composition may be a wettable powder, granules, dust, pellet or colloidal concentrate. Such dry forms may be formulated to dissolve immediately upon wetting or dissolve in a controlled-release, sustained-release, or other time-dependent manner. Also, the composition may be in a dry form that does not depend upon wetting or dissolving to be effective.

The compositions may additionally be provided in a formulation capable of spray. The spray may be a liquid or an aerosol.

The compositions may also be formulated in a nutritional composition (e.g. foodstuff, food additive, dietary supplement, or feed additive). For example, the compositions may be included in food products made using fermentation techniques such as wine, beer, and cheese.

A nutritional composition may include any of a variety of nutritional agents, which are well known in the art, including vitamins, minerals, essential and non-essential amino acids, carbohydrates, lipids, foodstuffs, dietary supplements, and the like. Thus, the compositions may include fiber, enzymes and other nutrients. Preferred fibers include, but are not limited to: psyllium, rice bran, oat bran, corn bran, wheat bran, fruit fiber and the like. Dietary or supplementary enzymes such as lactase, amylase, glucanase, catalase and the like can also be included. Typical vitamins are those, recommended for daily consumption and in the recommended daily amount (RDA).

In still another embodiment, it may be desirable to provide a coloring agent. Suitable color additives include, but are not limited to, food, drug and cosmetic colors (FD&C), drug and cosmetic colors (D&C), or external drug and cosmetic colors (Ext. D&C). Such colors may include but not be limited to red, yellow, green, blue, white, black, brown, purple, orange, any shade thereof, and any other color produced according to the above-named color formats.

The compositions stable under various conditions as a liquid or dry form. Preferably, the compositions are stable at room temperature.

### IV. Certain Embodiments

A microbial-based composition, comprising: a) a microorganism consortium in culture medium, wherein said microorganism consortium is co-cultured; b) at least one carbon source; and c) dechlorinated water.

The microbial-based composition of embodiment 1, wherein the microorganism consortium in culture medium comprises at least three microorganisms, wherein said at least five microorganisms comprise, at least, at least one sulfide-utilizing microorganism, at least two species of lactic acid bacteria, at least one Bacillus species, and at least one yeast.

The microbial-based composition of embodiment 1, wherein said microorganism consortium comprises at least two microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens.

The microbial-based composition of embodiment 1, wherein said microorganism consortium comprises at least three microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens.

The microbial-based composition of embodiment 1, wherein said microorganism consortium comprises at least four microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens.

The microbial-based composition of embodiment 1, wherein said microorganism consortium comprises at least five microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens.

The microbial-based composition of embodiment 1, wherein said microorganism consortium comprises at least six microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens.

The microbial-based composition of embodiment 1, wherein said microorganism consortium comprises at least seven microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens.

The microbial-based composition of embodiment 1, wherein said microorganism consortium comprises at least eight microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens.

The microbial-based composition of embodiment 1, wherein said microorganism consortium comprises at least nine microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens.

The microbial-based composition of embodiment 1, wherein said microorganism consortium comprises at least ten microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens.

The microbial-based composition of embodiment 1, wherein said microorganism consortium comprises at least eleven microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens.

The microbial-based composition of embodiment 1, wherein said microorganism consortium comprises at least twelve microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens.

The microbial-based composition of embodiment 1, wherein said microorganism consortium comprises at least thirteen microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens.

The microbial-based composition of embodiment 1, wherein said microorganism consortium comprises at least fourteen microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens.

The microbial-based composition of embodiment 1, wherein said microorganism consortium comprises at least fifteen microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens.

The microbial-based composition of embodiment 1, wherein said microorganism consortium comprises at least sixteen microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens.

The microbial-based composition of embodiment 1, wherein said microorganism consortium comprises at least seventeen microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens.

The microbial-based composition of embodiment 1, wherein said microorganism consortium comprises at least eighteen microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens.

The microbial-based composition of embodiment 1, wherein said microorganism consortium comprises at least ninteen microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens.

The microbial-based composition of embodiment 1, wherein said microorganism consortium comprises at least twenty microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens.

The microbial-based composition of embodiment 1, wherein said microorganism consortium comprises Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma vixens.

The microbial-based composition of embodiment 1, wherein said microorganism consortium comprises at least three microorganisms selected from the group consisting of purple non-sulfur bacteria, chromatianeae, green sulfur bacteria, colorless sulfur bacteria, filamentous green bacteria, and combinations thereof, and at least two microorganisms selected from the group consisting of Bacillus subtilis, Bifidobacterium animalis, Bifidobacterium bifidum, Bifidobacterium longum, Enterococcus lactis, Enterococcus thermophilus, Lactobacillus acidophilus, Lactobacillus bulgaricus, Lactobacillus casei, Lactobacillus fermentum, Lactobacillus plantarum, Rhodopseudomonas palustris, Rhodopseudomonas sphaeroides, and Saccharomyces cerevisiae.

The microbial-based composition of embodiment 1, wherein said microorganism consortium comprises, at least, Bacillus subtilis, a Lactobacillus sp., a Bifidobacterium sp., a Lactococcus sp., Streptococcus thermophilus, a purple non-sulfur bacteria sp., at least two non-pathogenic gram-positive Bacilli bacteria capable of fermentation, at least one fungus, and a yeast.

The microbial-based composition of embodiments 1-5, wherein said microorganism consortium comprises Bacillus subtilis, a Lactobacillus sp., a Bifidobacterium sp., a Lactococcus sp., Streptococcus thermophilus, a purple non-sulfur bacteria sp., at least two non-pathogenic gram-positive Bacilli bacteria capable of fermentation, and a yeast, and wherein said microorganism consortium is free of detectable non-Bacilli and non-Rhodospirillaceae bacteria.

The microbial-based composition of embodiments 1-6, further comprising the step of removing water, wherein said microbial-based composition product comprises <10% water (w/w).

The microbial-based composition of embodiment 1, wherein said microorganism consortium comprises at least five microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides and at least one yeast, and wherein said yeast is selected from the group consisting of Saccharomyces arboricolus, Saccharomyces bayanus, Saccharomyces boulardii, Saccharomyces bulderi, Saccharomyces cariocanus, Saccharomyces cariocus, Saccharomyces cerevisiae, Saccharomyces chevalieri, Saccharomyces dairenensis, Saccharomyces ellipsoideus, Saccharomyces eubayanus, Saccharomyces exiguus, Saccharomyces florentinus, Saccharomyces fragilis, Saccharomyces kluyveri, Saccharomyces kudriavzevii, Saccharomyces martiniae, Saccharomyces mikatae, Saccharomyces monacensis, Saccharomyces norbensis, Saccharomyces paradoxus, Saccharomyces pastorianus, Saccharomyces spencerorum, Saccharomyces turicensis, Saccharomyces unisporus, Saccharomyces uvarum, and Saccharomyces zonatus.

The microbial-based composition of embodiment 1, wherein said microorganism consortium comprises at least five microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, and at least one yeast, and wherein said yeast is selected from the group consisting of Saccharomyces cerevisiae CNCM 1-3060, Saccharomyces cerevisiae NCYC R397, Saccharomyces cerevisiae CNCM 1-3399, Saccharomyces cerevisiae NCYC R646, Saccharomyces cerevisiae CBS 493.9, Saccharomyces cerevisiae CNCM 1-1077, Saccharomyces cerevisiae NCYC Sc 47, Saccharomyces cerevisiae CNCM 1-4407, Saccharomyces cerevisiae MUCL 39885, Saccharomyces cerevisiae NCYC R404, Saccharomyces cerevisiae NCYC R404, Saccharomyces cerevisiae PCM KKP 2059p, Saccharomyces cerevisiae 140203, and Saccharomyces cerevisiae CNCM 1-1079.

The microbial-based composition of embodiment 1, wherein said microorganism consortium comprises at least five microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae and at least one Carnobacterium spp.

The microbial-based composition of embodiment 1, wherein said microorganism consortium comprises at least five microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae and at least two gram-positive Bacilli bacteria selected from the group consisting of Bifidobacterium actinocoloniiforme, Bifidobacterium adolescentis, Bifidobacterium angulatum, Bifidobacterium aquikefiri, Bifidobacterium asteroides, Bifidobacterium biavatii, Bifidobacterium bohemicum, Bifidobacterium bombi, Bifidobacterium boum, Bifidobacterium breve, Bifidobacterium callitrichos, Bifidobacterium catenulatum, Bifidobacterium choerinum, Bifidobacterium commune, Bifidobacterium coryneforme, Bifidobacterium cuniculi, Bifidobacterium crudilactis, Bifidobacterium denticolens, Bifidobacterium dentium, Bifidobacterium eulemuris, Bifidobacterium faecale, Bifidobacterium gallicum, Bifidobacterium gallinarum, Bifidobacterium hapali, Bifidobacterium indicum, Bifidobacterium inopinatum, Bifidobacterium kashiwanohense, Bifidobacterium infantis, Bifidobacterium lemurum, Bifidobacterium magnum, Bifidobacterium merycicum, Bifidobacterium minimum, Bifidobacterium mongoliense, Bifidobacterium moukalabense, Bifidobacterium myosotis, Bifidobacterium pseudocatenulatum, Bifidobacterium pseudolongum, Bifidobacterium psychraerophilum, Bifidobacterium pullorum, Bifidobacterium reuteri, Bifidobacterium ruminantium, Bifidobacterium saguini, Bifidobacterium scardovii, Bifidobacterium stellenboschense, Bifidobacterium stercoris, Bifidobacterium saeculare, Bifidobacterium subtile, Bifidobacterium thermacidophilum, Bifidobacterium thermophilum, Bifidobacterium tissieri, and Bifidobacterium tsurumiense.

The microbial-based composition of embodiment 1, wherein said microorganism consortium comprises at least five microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae and at least two gram-positive Bacilli bacteriaselected from the group consisting of Lactobacillus acidifarinae, Lactobacillus acidipiscis, Lactobacillus agilis, Lactobacillus algidus, Lactobacillus alimentarius, Lactobacillus amylolyticus, Lactobacillus amylophilus, Lactobacillus amylotrophicus, Lactobacillus amylovorus, Lactobacillus animalis, Lactobacillus antri, Lactobacillus apodemi, Lactobacillus aviarius, Lactobacillus bifermentans, Lactobacillus brevis, Lactobacillus buchneri, Lactobacillus camelliae, Lactobacillus catenaformis, Lactobacillus ceti, Lactobacillus coleohominis, Lactobacillus collinoides, Lactobacillus composti, Lactobacillus concavus, Lactobacillus coryniformis, Lactobacillus crispatus, Lactobacillus crustorum, Lactobacillus curvatus, Lactobacillus delbrueckii sub sp. bulgaricus, Lactobacillus delbrueckii sub sp. delbrueckii, Lactobacillus delbrueckii subsp. lactis, Lactobacillus dextrinicus, Lactobacillus diolivorans, Lactobacillus equi, Lactobacillus equigenerosi, Lactobacillus farraginis, Lactobacillus farciminis, Lactobacillus fornicalis, Lactobacillus fructivorans, Lactobacillus frumenti, Lactobacillus fuchuensis, Lactobacillus gallinarum, Lactobacillus gasseri, Lactobacillus gastricus, Lactobacillus ghanensis, Lactobacillus graminis, Lactobacillus hammesii, Lactobacillus hamsteri, Lactobacillus harbinensis, Lactobacillus hayakitensis, Lactobacillus helveticus, Lactobacillus hilgardii, Lactobacillus homohiochii, Lactobacillus finers, Lactobacillus ingluviei, Lactobacillus intestinalis, Lactobacillus jensenii, Lactobacillus johnsonii, Lactobacillus kalixensis, Lactobacillus kefiranofaciens, Lactobacillus kefiri, Lactobacillus kimchii, Lactobacillus kitasatonis, Lactobacillus kunkeei, Lactobacillus leichmannii, Lactobacillus lindneri, Lactobacillus malefermentans, Lactobacillus mali, Lactobacillus manihotivorans, Lactobacillus mindensis, Lactobacillus mucosae, Lactobacillus murinus, Lactobacillus nagelii, Lactobacillus namurensis, Lactobacillus nantensis, Lactobacillus oligofermentans, Lactobacillus oris, Lactobacillus panis, Lactobacillus pantheris, Lactobacillus parabrevis, Lactobacillus parabuchneri, Lactobacillus paracasei, Lactobacillus paracollinoides, Lactobacillus parakefiri, Lactobacillus paralimentarius, Lactobacillus paraplantarum, Lactobacillus pentosus, Lactobacillus perolens, Lactobacillus pontis, Lactobacillus protectus, Lactobacillus psittaci, Lactobacillus rennini, Lactobacillus reuteri, Lactobacillus rhamnosus, Lactobacillus rimae, Lactobacillus rogosae, Lactobacillus rossiae, Lactobacillus ruminis, Lactobacillus saerimneri, Lactobacillus sakei, Lactobacillus salivarius, Lactobacillus sanfranciscensis, Lactobacillus satsumensis, Lactobacillus secaliphilus, Lactobacillus sharpeae, Lactobacillus siliginis, Lactobacillus spicheri, Lactobacillus suebicus, Lactobacillus thailandensis, Lactobacillus ultunensis, Lactobacillus vaccinostercus, Lactobacillus vaginalis, Lactobacillus versmoldensis, Lactobacillus vini, Lactobacillus vitulinus, Lactobacillus zeae, and Lactobacillus zymae.

The microbial-based composition of embodiment 1, wherein said microorganism consortium comprises at least five microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae and at least two gram-positive Bacilli bacteria selected from the group consisting of Lactobacillus buchneri, Lactobacillus rhamnosis, Lactobacillus brevis, and Lactobacillus paracasei.

The microbial-based composition of embodiment 1, wherein said microorganism consortium comprises at least five microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Rhodopseudomonas palustris, Rhodopseudomonas sphaeroides, Saccharomyces cerevisiae and at least two gram-positive Bacilli bacteria is selected from the group consisting of Lactobacillus casei DSM 28872, Lactobacillus casei PCM B/00080, Lactobacillus casei DSM 28872, Lactobacillus plantarum KKP/593/p, Lactobacillus plantarum KKP/788/p, Lactobacillus plantarum PCM B/00081, Lactobacillus plantarum (NCIMB 41638), Lactobacillus plantarum DSM 29024, Lactobacillus plantarum (ATCC 55943), Lactobacillus plantarum (ATCC 55944), Lactobacillus plantarum (ATCC PTSA-6139), Lactobacillus plantarum (CNCM 1-3235), Lactobacillus plantarum DSM 11672, Lactobacillus plantarum (DSM 12836), Lactobacillus plantarum (DSM 12837), Lactobacillus plantarum (DSM 16565), Lactobacillus plantarum (DSM 16568), Lactobacillus plantarum (DSM 18112), Lactobacillus plantarum (DSM 18113), Lactobacillus plantarum (DSM 18114), Lactobacillus plantarum (DSM 19457), Lactobacillus plantarum (DSM 21762), Lactobacillus plantarum (DSM 23375), Lactobacillus plantarum (DSM 29025), Lactobacillus plantarum (DSM 3676), Lactobacillus plantarum (DSM 3677), Lactobacillus plantarum (DSM 8862), Lactobacillus plantarum (DSM 8866), Lactobacillus plantarum (LMG-21295), Lactobacillus plantarum (NCIMB 30083), Lactobacillus plantarum (NCIMB 30084), Lactobacillus plantarum (NCIMB 30084), Lactobacillus plantarum (NCIMB 30084), Lactobacillus plantarum (NCIMB 30236), Lactobacillus plantarum (NCIMB 41028), Lactobacillus plantarum (NCIMB 42150), Lactobacillus plantarum (VTT E-78076), Lactobacillus plantarum C KKP/788/p, Lactobacillus plantarum CECT 4528, Lactobacillus plantarum CECT 4528, Lactobacillus plantarum K KKP/593/p, Lactobacillus plantarum LP287, Lactobacillus plantarum LP329, Lactobacillus plantarum LP329, Lactobacillus plantarum NCIMB 30238, Lactobacillus buchneri KKP/907/p, Lactobacillus buchneri (DSM 22963), Lactobacillus buchneri (DSM 12856), Lactobacillus buchneri (DSM 13573), Lactobacillus buchneri CCM 1819, Lactobacillus buchneri (DSM 16774), Lactobacillus buchneri DSM 22501, Lactobacillus buchneri LN 40177, Lactobacillus buchneri LN4637, Lactobacillus buchneri LN 40177, Lactobacillus buchneri NCIMB 40788, Lactobacillus acidophilus CECT 4529, Lactobacillus acidophilus NBIMCC 8242, Lactobacillus rhamnosus (NCIMB 41640), Lactobacillus rhamnosus (NCIMB 30121), Lactobacillus rhamnosus DSM 29226, Lactobacillus rhamnosus DSM 7133, Lactobacillus rhamnosus (CNCM-I-3698), Lactobacillus rhamnosus ATCC 7469, Lactobacillus fermentum (NCIMB 41636), Lactobacillus brevis (DSM 12835), Lactobacillus brevis (DSM 21982), Lactobacillus brevis (DSM 12835), Lactobacillus brevis DSMZ 16680, Lactobacillus paracasei (DSM 16245), Lactobacillus paracasei (DSM 16773), and Lactobacillus paracasei NCIMB 30151.

The microbial-based composition of embodiment 1, wherein said microorganism consortium comprises at least five microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae and at least two gram-positive Bacilli bacteria selected from the group consisting of Lactococcus chungangensis, Lactococcus formosensis, Lactococcus fujiensis, Lactococcus garvieae, Lactococcus hircilactis, Lactococcus lactis, Lactococcus laudensis, Lactococcus nasutitermitis, Lactococcus piscium, Lactococcus raffinolactis, and Lactococcus taiwanensis.

The microbial-based composition of embodiment 1, wherein said microorganism consortium comprises at least five microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Bacillus licheniformis, and Bacillus amyloliquefaciens.

The microbial-based composition of embodiment 1, wherein said microorganism consortium comprises at least five microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae and at least two gram-positive Bacilli bacteria selected from the group consisting of Bacillus licheniformis (DSM 5749), Bacillus licheniformis DSM 28710, Bacillus licheniformis (DSM 19670), Bacillus licheniformis (DSM 21564), Bacillus licheniformis ATCC 53757, Bacillus amyloliquefaciens CECT 5940, Bacillus amyloliquefaciens (DSM 9553), Bacillus amyloliquefaciens (DSM 9554), Bacillus amyloliquefaciens (PTA-6507), Bacillus amyloliquefaciens (NRRL B-50013), Bacillus amyloliquefaciens (NRRL B-50104), Bacillus amyloliquefaciens SD80, and Bacillus amyloliquefaciens (ATCC 3978).

The microbial-based composition of embodiment 1, wherein said microorganism consortium comprises Bacillus subtilis, a Lactobacillus sp., a Bifidobacterium sp., a Lactococcus sp., Streptococcus thermophilus, at least one purple non-sulfur bacteria sp., at least two non-pathogenic gram-positive Bacilli bacteria capable of fermentation, and at least one yeast, and wherein at least one of said at least two gram-positive Bacilli bacteria is selected from the group consisting of Carnobacterium alterfunditum, Carnobacterium divergens, Carnobacterium funditium, Carnobacterium gallinarum, Carnobacterium iners, Carnobacterium inhibens, Carnobacterium jeotgali, Carnobacterium maltaromaticum, Carnobacterium mobile, Carnobacterium piscicola, Carnobacterium pleistocenium, and Carnobacterium viridans.

The microbial-based composition of embodiment 1, wherein said microorganism consortium comprises Bacillus subtilis, a Lactobacillus sp., a Bifidobacterium sp., a Lactococcus sp., Streptococcus thermophilus, at least one purple non-sulfur bacteria sp., at least two non-pathogenic gram-positive Bacilli bacteria capable of fermentation, at least one fungus, and at least one yeast, and wherein at least one of said at least two gram-positive Bacilli bacteria is Carnobacterium divergens PCM KKP 2012p.

The microbial-based composition of embodiment 1, wherein said microorganism consortium comprises Bacillus subtilis, a Lactobacillus sp., a Bifidobacterium sp., a Lactococcus sp., Streptococcus thermophilus, at least one purple non-sulfur bacteria sp., at least two non-pathogenic gram-positive Bacilli bacteria capable of fermentation, and at least one yeast, and wherein at least one of said at least two gram-positive Bacilli bacteria comprises Lactococcus lactis PCM B/00039, Lactobacillus casei PCM B/00080, Lactobacillus plantarum PCM B/00081, and Carnobacterium divergens PCM KKP 2012p, and said yeast comprises Saccharomyces cerevisiae PCM KKP 2059p.

The microbial-based composition of embodiment 1, wherein said microorganism consortium comprises Bacillus subtilis, a Lactobacillus sp., a Bifidobacterium sp., a Lactococcus sp., Streptococcus thermophilus, at least one purple non-sulfur bacteria sp., at least two non-pathogenic gram-positive Bacilli bacteria capable of fermentation, and at least one yeast, and wherein at least one of said at least two gram-positive Bacilli bacteria comprises Lactobacillus rhamnosus ATCC 7469, and said yeast comprises Saccharomyces cerevisiae IFO 0203.

The microbial-based composition of embodiment 1, wherein said microorganism consortium comprises Bacillus subtilis, Bacillus coagulans, at least one Lactobacillus sp., at least one Bifidobacterium sp., at least one Lactococcus sp., at least one Streptococcus sp., at least one purple non-sulfur bacteria sp., at least one acetic acid bacteria sp., at least two non-pathogenic gram-positive Bacilli bacteria capable of fermentation, and at least one yeast.

The microbial-based composition of embodiment 269, wherein said at least one Lactobacillus sp. is Lactobacillus acidophilus.

The microbial-based composition of embodiment 269, wherein said at least one Lactobacillus sp. is Lactobacillus bulgaricus.

The microbial-based composition of embodiment 269, wherein said at least one Lactobacillus sp. is Lactobacillus casei.

The microbial-based composition of embodiment 269, wherein said at least one Lactobacillus sp. is Lactobacillus fermentum.

The microbial-based composition of embodiment 269, wherein said at least one Lactobacillus sp. is Lactobacillus lactis.

The microbial-based composition of embodiment 269, wherein said at least one Lactobacillus sp. is Lactobacillus parafarraginis.

The microbial-based composition of embodiment 269, wherein said at least one Lactobacillus sp. is Lactobacillus plantarum.

The microbial-based composition of embodiment 269, wherein said at least one Lactobacillus sp. is Lactobacillus rapi.

The microbial-based composition of embodiment 269, wherein said at least one Bifidobacterium sp. is Bifidobacterium bifidum.

The microbial-based composition of embodiment 269, wherein said at least one Bifidobacterium sp. is Bifidobacterium longum.

The microbial-based composition of embodiment 269, wherein said Streptococcus sp. is Streptococcus thermophilus.

The microbial-based composition of embodiment 269, wherein said purple non-sulfur bacteria is Rhodopseudomonas palustris, Rhodobacter sphaeroides, or a combination thereof.

The microbial-based composition of embodiment 269, wherein said at least one acetic acid bacteria sp. is Acetobacter ghanensis.

The microbial-based composition of embodiment 269, wherein said at least one acetic acid bacteria sp. is Gluconacetobater diazotrophicus.

The microbial-based composition of embodiment 269, wherein said at least one yeast is selected from the group consisting of Saccharomyces arboricolus, Saccharomyces bayanus, Saccharomyces boulardii, Saccharomyces bulderi, Saccharomyces cariocanus, Saccharomyces cariocus, Saccharomyces cerevisiae, Saccharomyces chevalieri, Saccharomyces dairenensis, Saccharomyces elhpsoideus, Saccharomyces eubayanus, Saccharomyces exiguus, Saccharomyces florentinus, Saccharomyces fragilis, Saccharomyces kluyveri, Saccharomyces kudriavzevii, Saccharomyces martiniae, Saccharomyces mikatae, Saccharomyces monacensis, Saccharomyces norbensis, Saccharomyces paradoxus, Saccharomyces pastorianus, Saccharomyces spencerorum, Saccharomyces turicensis, Saccharomyces unisporus, Saccharomyces uvarum, and Saccharomyces zonatus.

The microbial-based composition of embodiment 269, wherein said at least one yeast is Saccharomyces cerevisiae.

The microbial-based composition of embodiment 1, wherein said microorganism consortium comprises Bacillis subtilis, Bacillus coagulans, Lactobacillus acidophilus, Lactobacillus bulgaricus, Lactobacillus casei, Lactobacillus fermentum, Lactobacillus lactis, Lactobacillus parafarraginis, Lactobacillus plantarum, Lactobacillus rapi, Bifidobacterium bifidum, Bifidobacterium longum, Streptococcus thermophilus, Rhodopseudomonas palustris, Rhodobacter sphaeroides, Acetobacter ghanensis, Gluconacetobater diazotrophicus, and Saccharomyces cerevisiae.

The microbial-based composition of embodiment 1, further comprising fulvic acid.

The microbial-based composition of embodiment 1, further comprising humic acid.

The microbial-based composition of embodiment 1, further comprising propionic acid.

The microbial-based composition of embodiment 1, further comprising citric acid.

The microbial-based composition of embodiment 1, further comprising homo citric acid.

The microbial-based composition of embodiment 1, further comprising ascorbic acid.

The microbial-based composition of embodiment 1, further comprising lactic acid.

The microbial-based composition of embodiment 1, further comprising oxalic acid.

The microbial-based composition of embodiment 1, further comprising vanillic acid.

The microbial-based composition of embodiment 1, further comprising gallic acid.

The microbial-based composition of embodiment 1, further comprising malate.

The microbial-based composition of embodiment 1, further comprising gluconic acid.

The microbial-based composition of embodiment 1, further comprising 2,3-dihydroxybenzoic acid.

The microbial-based composition of embodiment 1, further comprising 1,4,7-tetra azacyclododecane 1,4,7,10-tetra acetic acid.

The microbial-based composition of embodiment 1, further comprising ethylene diamine tetraacetic acid.

The microbial-based composition of embodiment 1, further comprising ethylene diamine-N, N′-disuccinic acid.

The microbial-based composition of embodiment 1, further comprising pentetic acid.

The microbial-based composition of embodiment 1, further comprising nitrilotriacetic acid.

The microbial-based composition of embodiment 1, further comprising a dicarboxylic acid having at least one β-hydroxyl group.

The microbial-based composition of embodiment 1, further comprising a tricarboxylic acid having at least one β-hydroxyl group.

The microbial-based composition of embodiment 1, further comprising a phenolic acid having at least one ortho-hydroxyl group.

The microbial-based composition of embodiment 1, further comprising an organic acid configured to chelate cobalt.

The microbial-based composition of embodiment 1, further comprising an organic acid configured to chelate iron.

The microbial-based composition of embodiment 1, further comprising an organic acid configured to chelate selenium.

The microbial-based composition of embodiment 1, further comprising an organic acid configured to chelate silicon.

The microbial-based composition of embodiment 1, further comprising a nitrogen source, a potassium source, a chelated metal, and a salt.

The microbial-based composition of any embodiment, wherein said nitrogen source is a protein hydrolysate.

The microbial-based composition of any embodiment, wherein said nitrogen source is a collagen-derived protein hydrolysate.

The microbial-based composition of any embodiment, wherein said nitrogen source is soybeans.

The microbial-based composition of any embodiment, wherein said nitrogen source is soybean hydrolysate.

The microbial-based composition of any embodiment, wherein said nitrogen source is peas.

The microbial-based composition of any embodiment, wherein said nitrogen source is pea protein hydrolysate.

The microbial-based composition of any embodiment, wherein said nitrogen source is whey.

The microbial-based composition of any embodiment, wherein said nitrogen source is whey protein hydrolysate.

The microbial-based composition of any embodiment, wherein said nitrogen source is rice.

The microbial-based composition of any embodiment, wherein said nitrogen source is rice protein hydrolysate.

The microbial-based composition of any embodiment, wherein said nitrogen source is alfalfa.

The microbial-based composition of any embodiment, wherein said nitrogen source is alfalfa protein hydrolysate.

The microbial-based composition of any embodiment, wherein said nitrogen source is legume seeds.

The microbial-based composition of any embodiment, wherein said nitrogen source is legume seed protein hydrolysate.

The microbial-based composition of any embodiment, wherein said nitrogen source is corn.

The microbial-based composition of any embodiment, wherein said nitrogen source is corn protein hydrolysate.

The microbial-based composition of any embodiment, wherein said nitrogen source is casein.

The microbial-based composition of any embodiment, wherein said nitrogen source is casein protein hydrolysate.

The microbial-based composition of any embodiment, wherein said nitrogen source is yeast extract.

The microbial-based composition of any embodiment, wherein said nitrogen source is tryptone.

The microbial-based composition of any embodiment, wherein said nitrogen source is beef extract.

The microbial-based composition of any embodiment, wherein said nitrogen source is peptone.

The microbial-based composition of any embodiment, wherein said nitrogen source is potassium nitrate.

The microbial-based composition of any embodiment, wherein said nitrogen source is ammonium nitrate.

The microbial-based composition of any embodiment, wherein said nitrogen source is ammonium chloride.

The microbial-based composition of any embodiment, wherein said nitrogen source is ammonium sulfate.

The microbial-based composition of any embodiment, wherein said nitrogen source is ammonium phosphate.

The microbial-based composition of any embodiment, wherein said nitrogen source is ammonia.

The microbial-based composition of any embodiment, wherein said nitrogen source is distiller's dried grains with solubles (DDGS).

The microbial-based composition of any embodiment, wherein said nitrogen source is urea.

The microbial-based composition of any embodiment, wherein said potassium source is potassium oxide.

The microbial-based composition of any embodiment, wherein said potassium source is potassium citrate.

The microbial-based composition of any embodiment, wherein said potassium source is potassium sulfate.

The microbial-based composition of any embodiment, wherein said potassium source is monopotassium phosphate.

The microbial-based composition of any embodiment, wherein said potassium source is potassium dihydrogen phosphate.

The microbial-based composition of any embodiment, wherein said potassium source is potassium nitrate.

The microbial-based composition of any embodiment, wherein said potassium source is potassium chloride.

The microbial-based composition of any embodiment, wherein said potassium source is ammonium phosphate.

The microbial-based composition of any embodiment, wherein said chelated metal is iron EDDHA.

The microbial-based composition of any embodiment, wherein said chelated metal is manganese EDTA.

The microbial-based composition of any embodiment, wherein said chelated metal is zinc EDTA.

The microbial-based composition of any embodiment, wherein said salt is boric acid.

The microbial-based composition of any embodiment, wherein said salt is disodium octab orate tetrahadrate.

The microbial-based composition of any embodiment, wherein said salt is sodium molybdate.

The microbial-based composition of any embodiment, wherein said salt is ferrous sulfate.

The microbial-based composition of any embodiment, wherein said salt is manganese sulfate.

The microbial-based composition of any embodiment, wherein said salt is zinc sulfate.

The microbial-based composition of embodiment 1, further comprising a second additive, wherein said second additive comprises protein hydrolysate, potassium citrate, disodium octaborate tetrahydrate, ferrous sulfate, manganese sulfate, and zinc sulfate.

The microbial-based composition of embodiment 113, wherein said second additive comprises 2-25% (w/w) protein hydrolysate, 5-25% (w/w) potassium citrate, 1-10% (w/w) disodium octaborate tetrahydrate, 1-10% (w/w) ferrous sulfate, 1-10% (w/w) manganese sulfate, and 1-10% zinc sulfate.

The microbial-based composition of embodiment 113, wherein said second additive comprises 2-15% (w/w) protein hydrolysate, 5-10% (w/w) potassium citrate, 1-5% (w/w) disodium octaborate tetrahydrate, 1-5% (w/w) ferrous sulfate, 1-5% (w/w) manganese sulfate, and 1-5% zinc sulfate.

The microbial-based composition of embodiment 113, wherein said second additive comprises 5.0% (w/w) protein hydrolysate, 8.0% (w/w) potassium citrate, 0.6% (w/w) disodium octab orate tetrahydrate, 1.6% (w/w) ferrous sulfate, 1.6% (w/w) manganese sulfate, and 1.6% zinc sulfate.

The microbial-based composition of embodiment 1, wherein said carbon source is molasses.

The microbial-based composition of embodiment 1, wherein said carbon source is rum.

The microbial-based composition of embodiment 1, wherein said carbon source is starch.

The microbial-based composition of embodiment 1, wherein said carbon source is yeast extract.

The microbial-based composition of embodiment 1, wherein said carbon source comprises at least one amino acid.

The microbial-based composition of embodiment 1, wherein said carbon source is glucose.

The microbial-based composition of embodiment 1, wherein said carbon source is arabinose.

The microbial-based composition of embodiment 1, wherein said carbon source is mannose.

The microbial-based composition of embodiment 1, wherein said carbon source is glucosamine.

The microbial-based composition of embodiment 1, wherein said carbon source is maltose.

The microbial-based composition of embodiment 1, wherein said carbon source is sugar cane.

The microbial-based composition of embodiment 1, wherein said carbon source is ethanol.

The microbial-based composition of embodiment 1, wherein said carbon source is glycerol.

The microbial-based composition of embodiment 1, wherein said carbon source is fumaric acid.

The microbial-based composition of embodiment 1, wherein said carbon source is acetic acid.

The microbial-based composition of embodiment 1, wherein said carbon source is citric acid.

The microbial-based composition of embodiment 1, wherein said carbon source is malic acid.

The microbial-based composition of embodiment 1, wherein said carbon source is gluconic acid.

The microbial-based composition of embodiment 1, wherein said carbon source is propionic acid.

The microbial-based composition of embodiment 1, wherein said carbon source is pyruvic acid.

The microbial-based composition of embodiment 1, wherein said carbon source is malonic acid.

The microbial-based composition of embodiment 1, wherein said carbon source is soybean oil.

The microbial-based composition of embodiment 1, wherein said carbon source is rice bran oil.

The microbial-based composition of embodiment 1, wherein said carbon source is olive oil.

The microbial-based composition of embodiment 1, wherein said carbon source is corn oil.

The microbial-based composition of embodiment 1, wherein said carbon source is sesame oil.

The microbial-based composition of embodiment 1, further comprising at least one natural oil.

The microbial-based composition of embodiment 1, further comprising African lemon bush oil.

The microbial-based composition of embodiment 1, further comprising anise oil.

The microbial-based composition of embodiment 1, further comprising bay oil.

The microbial-based composition of embodiment 1, further comprising bergamot oil.

The microbial-based composition of embodiment 1, further comprising boronia oil.

The microbial-based composition of embodiment 1, further comprising canola oil.

The microbial-based composition of embodiment 1, further comprising carrot oil.

The microbial-based composition of embodiment 1, further comprising cassia oil.

The microbial-based composition of embodiment 1, further comprising catnip oil.

The microbial-based composition of embodiment 1, further comprising cedarwood oil.

The microbial-based composition of embodiment 1, further comprising chamomile oil.

The microbial-based composition of embodiment 1, further comprising cinnamon oil.

The microbial-based composition of embodiment 1, further comprising citronella oil.

The microbial-based composition of embodiment 1, further comprising clary sage oil.

The microbial-based composition of embodiment 1, further comprising clove oil.

The microbial-based composition of embodiment 1, further comprising cypress oil.

The microbial-based composition of embodiment 1, further comprising eucalyptus oil.

The microbial-based composition of embodiment 1, further comprising galbanum oil.

The microbial-based composition of embodiment 1, further comprising garlic oil.

The microbial-based composition of embodiment 1, further comprising ginger oil.

The microbial-based composition of embodiment 1, further comprising geranium oil.

The microbial-based composition of embodiment 1, further comprising grapefruit oil.

The microbial-based composition of embodiment 1, further comprising hazelnut oil.

The microbial-based composition of embodiment 1, further comprising jasmine oil.

The microbial-based composition of embodiment 1, further comprising jojoba oil.

The microbial-based composition of embodiment 1, further comprising lavender oil.

The microbial-based composition of embodiment 1, further comprising lemon oil.

The microbial-based composition of embodiment 1, further comprising lime oil.

The microbial-based composition of embodiment 1, further comprising mandarin oil.

The microbial-based composition of embodiment 1, further comprising nutmeg oil.

The microbial-based composition of embodiment 1, further comprising orange oil.

The microbial-based composition of embodiment 1, further comprising palma rosa oil.

The microbial-based composition of embodiment 1, further comprising patchouli oil.

The microbial-based composition of embodiment 1, further comprising Peru balsams.

The microbial-based composition of embodiment 1, further comprising peppermint oil.

The microbial-based composition of embodiment 1, further comprising rosemary oil.

The microbial-based composition of embodiment 1, further comprising rosewood oil.

The microbial-based composition of embodiment 1, further comprising sage oil.

The microbial-based composition of embodiment 1, further comprising sandalwood oil.

The microbial-based composition of embodiment 1, further comprising spear mint oil.

The microbial-based composition of embodiment 1, further comprising star anise oil.

The microbial-based composition of embodiment 1, further comprising tea tree oil.

The microbial-based composition of embodiment 1, further comprising tangerine oil.

The microbial-based composition of embodiment 1, further comprising thyme oil.

The microbial-based composition of embodiment 1, further comprising verbena oil.

The microbial-based composition of embodiment 1, further comprising white clover oil.

The microbial-based composition of embodiment 1, further comprising ylang ylang oil.

The microbial-based composition of embodiment 1, wherein the microbial-based composition comprises said microorganism consortium at 40-80% (w/w), said carbon source at 1-20% (w/w), and dechlorinated water at 10-40% (w/w).

The microbial-based composition of embodiment 1, wherein the microbial-based composition comprises said microorganism consortium at 50-70% (w/w), said carbon source at 5-15% (w/w), and dechlorinated water at 10-30% (w/w).

The microbial-based composition of embodiment 1, wherein the microbial-based composition comprises said microorganism consortium at 60% (w/w), said carbon source at 20% (w/w), and dechlorinated water at 20% (w/w).

The microbial-based composition of embodiment 1, wherein the microbial-based composition comprises: a) said microorganism consortium at 40-80% (w/w), wherein said microorganism consortium comprises Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens; b) said carbon source at 1-20% (w/w); and c) dechlorinated water at 10-40% (w/w).

The microbial-based composition of embodiment 1, wherein the microbial-based composition comprises: a) said microorganism consortium at 50-70% (w/w), wherein said microorganism consortium comprises Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens; b) said carbon source at 5-15% (w/w); and c) dechlorinated water at 10-30% (w/w).

The microbial-based composition of embodiment 1, wherein the microbial-based composition comprises: a) said microorganism consortium at 60% (w/w), wherein said microorganism consortium Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Gluconacetobacter diazotrophicus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma virens; b) said carbon source at 10% (w/w); and c) dechlorinated water at 20% (w/w).

A microbial-based composition of any embodiment or use as an input to enhance plant health, plant growth, plant yield, or plant product quality.

A microbial-based composition of any embodiment produced by combining in any order said at least three microorganisms into said culture media and fermenting for at least 24 hours or from about 24 hours to about 30 days.

A method of enhancing the health of a plant, comprising administering a therapeutic amount of a finished product to a recipient plant or seed, wherein the finished product comprises a microbial-based composition comprised of: a) a microorganism consortium comprised of at least five microorganisms, wherein said at least five microorganisms are selected according to any of the preceding embodiments; b) at least one carbon source; and c) dechlorinated water.

The method of embodiment 197, wherein the microorganism consortium is fermented for at least fifteen days.

The method of embodiment 197, further comprising an essential oil selected from the group consisting of Lippia javanica oil, anise oil, bay oil, bergamot oil, boronia oil, canola oil, carrot oil, cassia oil, catnip oil, cedarwood oil, chamomile oil, cinnamon oil, citronella oil, clary sage oil, clove oil, cypress oil, eucalyptus oil, galbanum oil, garlic oil, ginger oil, geranium oil, grapefruit oil, hazelnut oil, jasmine oil, jojoba oil, lavender oil, lavandin oil, lemon oil, lime oil, mandarin oil, nutmeg oil, orange oil, palma rosa oil, patchouli oil, Peru balsams, peppermint oil, rosemary oil, rosewood oil, sage oil, sandalwood oil, spear mint oil, star anise oil, tea tree oil, tangerine oil, thyme oil, tolu, verbena oil, white clover oil, and ylang ylang oil.

The method of embodiment 197, wherein the finished product is applied to a foliar surface of a plant.

The method of embodiment 1977, wherein the finished product is applied to at least one area of plant growing medium surrounding at least one root of a plant.

The method of any of the preceding embodiments, wherein said plant is selected from the group consisting of cannabis sativa, cannabis indica, and cannabis ruderalis.

The method of any of the preceding embodiments, wherein said plant is selected from the group consisting of corn, wheat, barley, oats and rice.

The method of any of the preceding embodiments, wherein said plant is selected from the group consisting of amaranth, beet greens, bitterleaf, bok choy, Brussels sprout, cabbage, catsear, celtuce, choukwee, Ceylon spinach, chicory, Chinese mallow, chrysanthemum leaf, corn salad, cress, dandelion, endive, epazote, fat hen, fiddlehead, fluted pumpkin, golden samphire, Good King Henry, ice plant, jambu, kai-lan, kale, komatsuna, kuka, Lagos bologi, land cress, lettuce, lizard's tail, melokhia, mizuna greens, mustard, Chinese cabbage, New Zealand spinach, orache, pea leaf, polk, radicchio, rocket (arugula), samphire, sea beet, seakale, Sierra Leone bologi, soko, sorrel, spinach, summer purslane, Swiss chard, tatsoi, turnip greens, watercress, water spinach, winter purslane and you choy, flowering and fruiting vegetables such as acorn squash, Armenian cucumber, avocado, bell pepper, bitter melon, butternut squash, caigua, Cape gooseberry, cayenne pepper, chayote, chili pepper, cucumber, eggplant (aubergine), globe artichoke, luffa, Malabar gourd, parwal, pattypan squash, perennial cucumber, pumpkin, snake gourd, squash (marrow), sweetcorn, sweet pepper, tinda, tomato, tomatillo, winter melon, West Indian gherkin and zucchini (courgette), podded vegetables (legumes) such as American groundnut, azuki bean, black bean, black-eyed pea, chickpea (garbanzo bean), drumstick, dolichos bean, fava bean (broad bean), French bean, guar, haricot bean, hemp, horse gram, Indian pea, kidney bean, lentil, lima bean, marijuana, moth bean, mung bean, navy bean, okra, pea, peanut (groundnut), pigeon pea, pinto bean, rice bean, runner bean, soybean, tarwi, tepary bean, urad bean, velvet bean, winged bean and yardlong bean, bulb and stem vegetables such as asparagus, cardoon, celeriac, celery, elephant garlic, fennel, garlic, kohlrabi, kurrat, leek, lotus root, nopal, onion, Prussian asparagus, shallot, Welsh onion and wild leek, root and tuber vegetables, such as ahipa, arracacha, bamboo shoot, beetroot, black cumin, burdock, broadleaf arrowhead, camas, canna, carrot, cassava, Chinese artichoke, daikon, earthnut pea, elephant-foot yam, ensete, ginger, gobo, Hamburg parsley, horseradish, Jerusalem artichoke, jicama, parsnip, pignut, plectranthus, potato, prairie turnip, radish, rutabaga (swede), salsify, scorzonera, skirret, sweet potato, taro, ti, tigernut, turnip, ulluco, wasabi, water chestnut, yacon and yam, and herbs, such as angelica, anise, basil, bergamot, caraway, cardamom, chamomile, chives, cilantro, coriander, dill, fennel, ginseng, jasmine, lavender, lemon balm, lemon basil, lemongrass, marjoram, mint, oregano, parsley, poppy, saffron, sage, star anise, tarragon, thyme, turmeric and vanilla.

The method of any of the preceding embodiments, wherein said plant is selected from the group consisting of apple, apricot, banana, blackberry, blackcurrant, blueberry, boysenberry, cantaloupe, cherry, citron, clementine, cranberry, damson, dragonfruit, fig, grape, grapefruit, greengage, gooseberry, guava, honeydew, jackfruit, key lime, kiwifruit, kumquat, lemon, lime, loganberry, longan, loquat, mandarin, mango, mangosteen, melon, muskmelon, orange, papaya, peach, pear, persimmon, pineapple, plantain, plum, pomelo, prickly pear, quince, raspberry, redcurrant, starfruit, strawberry, tangelo, tangerine, tayberry, ugli fruit and watermelon.

The method of any of the preceding embodiments, wherein said plant is selected from the group consisting of barley, corn (maize), millet, oats, rice, rye, sorghum (milo) and wheat), non-gramineous seed crops such as buckwheat, cotton, flaxseed (linseed), mustard, poppy, rapeseed (including canola), safflower, sesame and sunflower.

The method of any of the preceding embodiments, wherein said plant is selected from the group consisting of barley, corn, millet, oats, rice, rye, sorghum, and wheat.

The method of any of the preceding embodiments, wherein said plant is selected from the group consisting of hay, oats, corn, grain sorghum, Bermuda grass, star grass, limpograss, forage sorghum, pearl millet, ryegrass, alfalfa, red clover, hairy indigo, alyce clover, shamrock, vicia sativa, aeschynomene, and rhizoma perennial peanut.

The method of any of the preceding embodiments, wherein said plant is selected from the group consisting of sugar beet, sugar cane, hops, and tobacco.

A method for preserving silage quality and reducing damage to a cut plant product from at least one plant pathogen, comprising administering a therapeutic amount of a finished product to the at least one cut plant product, wherein said finished product comprises a microbial-based composition comprised of: a) a microorganism consortium comprised of at least five microorganisms, wherein said at least five microorganisms are selected according to any of the preceding embodiments; b) at least one carbon source; and c) dechlorinated water.

The method of any of the preceding embodiments, wherein said cut plant product is selected from the group consisting of silage, oatlage, and haylage.

The method of any of the preceding embodiments, wherein said cut plant product is selected from the group consisting of corn, grain sorghum, Bermuda grass, star grass, limpograss, forage sorghum, pearl millet, ryegrass, alfalfa, red clover, hairy indigo, alyce clover, shamrock, vicia sativa, aeschynomene, and rhizoma perennial peanut.

The method of any of the preceding embodiments, wherein the finished product is administered to a cut plant product at least once during at least one stage of a silage process, wherein the at least one stage is selected from the group consisting of a respiration phase, a fermentation phase, a feedlot management stage phase, a cellular respiration phase, a lactic acid production phase, an acetic acid production phase, a lactic acid formation phase, a lactic acid fermentation phase, an aerobic decomposition phase, an aerobic phase, a lag phase, a fermentation phase, and a stable phase.

The method of any of the preceeding embodiments, wherein the microorganism consortium is fermented for at least twenty-four hours.

The method of any of the preceeding embodiments, wherein the microorganism consortium is fermented for at least one day.

The method of any of the preceeding embodiments, wherein the microorganism consortium is fermented for at least two days.

The method of any of the preceeding embodiments, wherein the microorganism consortium is fermented for at least three days.

The method of any of the preceeding embodiments, wherein the microorganism consortium is fermented for at least four days.

The method of any of the preceeding embodiments, wherein the microorganism consortium is fermented for at least five days.

The method of any of the preceeding embodiments, wherein the microorganism consortium is fermented for at least six days.

The method of any of the preceeding embodiments, wherein the microorganism consortium is fermented for at least seven days.

The method of any of the preceeding embodiments, wherein the microorganism consortium is fermented for at least eight days.

The method of any of the preceeding embodiments, wherein the microorganism consortium is fermented for at least nine days.

The method of any of the preceeding embodiments, wherein the microorganism consortium is fermented for at least ten days.

The method of any of the preceeding embodiments, wherein the microorganism consortium is fermented for at least eleven days.

The method of any of the preceeding embodiments, wherein the microorganism consortium is fermented for at least twelve days.

The method of any of the preceeding embodiments, wherein the microorganism consortium is fermented for at least thirteen days.

The method of any of the preceeding embodiments, wherein the microorganism consortium is fermented for at least fourteen days.

The method of any of the preceeding embodiments, wherein the microorganism consortium is fermented for at least fifteen days.

The method of any of the preceeding embodiments, wherein the microorganism consortium is fermented for at least sixteen days.

The method of any of the preceeding embodiments, wherein the microorganism consortium is fermented for at least seventeen days.

The method of any of the preceeding embodiments, wherein the microorganism consortium is fermented for at least eighteen days.

The method of any of the preceeding embodiments, wherein the microorganism consortium is fermented for at least ninteen days.

The method of any of the preceeding embodiments, wherein the microorganism consortium is fermented for at least twenty days.

The method of any of the preceeding embodiments, wherein the microorganism consortium is fermented for at least twenty-one days.

The method of any of the preceeding embodiments, wherein the microorganism consortium is fermented for at least twenty-two days.

The method of any of the preceeding embodiments, wherein the microorganism consortium is fermented for at least twenty-three days.

The method of any of the preceeding embodiments, wherein the microorganism consortium is fermented for at least twenty-four days.

The method of any of the preceeding embodiments, wherein the microorganism consortium is fermented for at least twenty-five days.

The method of any of the preceeding embodiments, wherein the microorganism consortium is fermented for at least twenty-six days.

The method of any of the preceeding embodiments, wherein the microorganism consortium is fermented for at least twenty-seven days.

The method of any of the preceeding embodiments, wherein the microorganism consortium is fermented for at least twenty-eight days.

The method of any of the preceeding embodiments, wherein the microorganism consortium is fermented for at least twenty-nine days.

The method of any of the preceeding embodiments, wherein the microorganism consortium is fermented for at least thirty days.

The method of any of the preceeding embodiments, wherein the microorganism consortium is fermented for no more than thirty days.

### V. Definitions

Unless defined otherwise, all technical and scientific terms used herein have the same meaning as is commonly understood by one of ordinary skill in the art. All patents, applications, published applications and other publications are incorporated by reference in their entirety. In the event that there is a plurality of definitions for a term herein, those in this section prevail unless stated otherwise.

For clarity, in some embodiments, the composition may be referred to herein as a “microbial-based composition,” a “microorganism consortium,” a “microorganism consortium,” or simply the “composition.” As well, the terms “microorganism” and “microbes” may at times be used interchangeably herein. All elements provided herein are to be understood as being preceded by the descriptor “at least one,” unless otherwise noted. However, for simplicity, at times the descriptor “at least one” may be omitted. Therefore, any item denoted in the singular or not specifically designated as “at least one” or “one or more” should be understood to be provided by the present disclosure in at least one amount or iteration. For example, the term “first adjuvant” should be understood as “at least one first adjuvant,” and so forth.

The term “about” means within the inclusive range of ±15% of the numerical value it modifies.

As used herein, “administering” is used in its broadest sense to mean contacting a subject, surface, liquid, or environment with a composition of the disclosure.

The term “agriculturally acceptable” applied to a material or composition herein means not unacceptably damaging or toxic to a plant or its environment, and not unsafe to the user or others that may be exposed to the material when used as described herein.

The term “co-culture” refers to a culture of microorganisms that includes at least two microorganisms, described herein.

A “foliar surface” herein is typically a leaf surface, but other green parts of plants have surfaces that may permit absorption of active ingredient, including petioles, stipules, stems, bracts, flowerbuds, etc., and for present purposes “foliar surfaces” will be understood to include surfaces of such green parts.

The term “insecticidally-effective amount” refers to an amount of the composition that can bring about death to at least one insect, or to noticeably reduce insect growth, feeding, or normal physiological development. This amount will vary depending on such factors as, for example, the specific target insects to be controlled, the specific environment, location, plant, crop, or agricultural site to be treated, the environmental conditions, and the method, rate, concentration, stability, and quantity of application. The formulations may also vary with respect to climatic conditions, environmental considerations, frequency of application, and severity of insect infestation.

The term “detectable” as used herein means at or above the limit of detection of a colony-forming unit of particular bacteria, yeast, or other microbes in a microorganism consortium. Methods for determining the presence of (detecting) a particular species or strain of microbe include culture techniques and non-culture techniques. Culture techniques include the use of selective agar media and determination of colony morphology, such as e.g., LAMVAB and Rogosa agar for Lactobacilli ssp., liver-cysteine-lactose and raffinose for Bifidobacterium ssp., heterotrophic plate counting, and the like. Non-culture techniques include e.g., flow cytometry, direct epifluorescent counting, PCR and other DNA-based methods, such as quantitative PCR, and metabolic/chemical methods, such as propridium monoazide PCR or ethidium monoazide PCR. Methods of detection useful in the practice of this disclosure are described in Catherine Davis, “Enumeration of microbial-based composition strains: Review of culture-dependent and alternative techniques to quantify viable bacteria,” Journal of Microbiological Methods, Volume 103, 2014, pp. 9-17; Jackson and Bird, “Comparison of two selective media for the detection and enumeration of Lactobacilli in human faeces,” Journal of Microbiological Methods 51 (2002) 313-321; Lu et al., “Fine Structure of Tibetan Kefir Grains and Their Yeast Distribution, Diversity, and Shift,” PLoS One. 2014; 9(6): e101387; Rachbid et al., “Assessment of the microbial diversity of Brazilian kefir grains by PCR-DGGE and pyrosequencing analysis,” Food Microbiology, Volume 31, Issue 2, September 2012, Pages 215-221; Furet et al., “Molecular quantification of lactic acid bacteria in fermented milk products using real-time quantitative PCR,” International Journal of Food Microbiology, Volume 97, Issue 2, 15 Dec. 2004, Pages 197-207; and Garcia-Cayuel et al., “Simultaneous detection and enumeration of viable lactic acid bacteria and bifidobacteria in fermented milk by using propidium monoazide and real-time PCR,” International Dairy Journal, Volume 19, Issues 6-7, June-July 2009, Pages 405-409.

The phrase “fermentation medium” may refer to a mixture including at least one microorganism, expression products of the microorganism(s), substances produced by the microorganisms, extracts of the microorganisms, and a culture medium or other elements of a culture medium. In some embodiments, the expression product or substance produced by a microorganism may comprise a gas or organic compound product of the fermentation of a carbohydrate, such as an organic acid like acetic acid, citric acid, gluconic acid, lactic acid, propionic acid, pyruvic acid, or succinic acid, a gas like carbon dioxide, or an alcohol like ethanol. In a preferred embodiment, the expression product or substance produced by a microorganism is lactic acid, ethanol, or a combination of lactic acid and ethanol.

“Fermentation product” refers to both or either organic chemical products of a fermentation reaction (e.g., carbon dioxide, ethanol, lactic acid, propionic acid), and/or the fermented carbon source (e.g., fermented sugar cane molasse, fermented rice bran).

The term “finished product” refers to a mixture including a fermentation product. The finished product may include additional additives. Organic fish fertilizer is a finished product.

A “locus” as used herein is inclusive of a foliar surface and also includes an area in proximity to a plant or the area in which a plurality of seed is or can be sown.

The phrase “non-pathogenic gram-positive Bacilli bacteria” refers to those bacteria that belong to the Bacilli taxonomic class of bacteria that contains two orders, Bacillales and Lactobacillales. The term “gram-positive” is used herein to distinguish the subject Bacilli from the group of gram-negative rod-shaped bacteria that are sometimes referred to as bacilli, including for example Escherichia coli and other coliform bacteria. The term “non-pathogenic” is used to refer to those Bacilli that do not cause disease or harm to a plant or animal, i.e., excluding pathogenic bacteria. For example, certain species of Bacillus (e.g., anthraces), Listeria, Staphylococcus, and Streptococcus are pathogenic and are excluded. Non-limiting examples of non-pathogenic gram-positive Bacilli bacteria include Bifidobacterium spp., Lactobacillus spp., Lactococcus spp., Carnobacterium spp., Streptococcus spp., and Bacillus spp.

The term “microorganism consortium” refers to a composition that contains a live beneficial or useful microorganism alone, in combination with another microorganism, and/or combined with other ingredients such as e.g., energy sources, pre-biotics, stabilizers, and the like, in culture media. Beneficial microorganisms are generally known in the art and include such bacteria as lactic acid fermenting (obligative and facultative) bacteria, phototrophic bacteria, and non-pathogenic bacilli, as well as fermenting yeast such as the Saccharomycetaceae. Microorganism consortiums are generally known to be useful in ameliorating gut flora, remediating wastewater, treating microbial imbalances in animals and plants, protecting animals, plants, and soil from harmful microbes, and improving food animal and food plant production and yield. Useful microbial-based composition microorganisms can be found listed for example in Microbial-based composition Bacteria: Fundamentals, Therapy, and Technological Aspects, edited by J. Paulo Sousa e Silva, Ana Cristina Freitas, CRC Press, Apr. 2, 2014; and The European Union Register of Feed Additives pursuant to Regulation (EC) No 1831/2003, Annex I: List of additives, available at https://ec.europa.eu/food/sites/food/files/safety/docs/animal-feed-eu-reg comm_register_feed_additives 1831-03.pdf.

The term “physiologically acceptable carrier” refers to a carrier or a diluent that does not cause significant irritation to a subject and does not abrogate the biological activity and properties of the administered composition.

The term “excipient” refers to an inert substance added to a pharmaceutical composition to further facilitate administration of a composition. Examples, without limitation, of excipients include calcium carbonate, calcium phosphate, various sugars and types of starch, cellulose derivatives, gelatin, vegetable oils and polyethylene glycols.

The term “seed” as used herein, is not limited to any particular type of seed and can refer to seed from a single plant species, a mixture of seed from multiple plant species, or a seed blend from various strains within a plant species. The disclosed compositions can be utilized to treat gymnosperm seed, dicotyledonous angiosperm seed and monocotyledonous angiosperm seed.

The terms “seed coating” or “seed dressing” as used herein refers generally to a coating or matrix formed on at least part of the seed, the coating or matrix containing or comprising the at least one AI. Optional compounds or agents may be included in the seed coating to facilitate the seed coating process or the disintegration/releasing of the at least one AI from the coating, or to prevent excessive dust-off or to add color to the treated seed.

The term “seed treatment” as used herein refers generally to contacting a seed with a compound or composition of matter containing or comprising at least one active ingredient (a.i. or AI). The compound or composition of matter may be in any form suitable to the seed, for example, liquid, gel, emulsion, suspension, dispersion, spray, or powder. Seed treatment is inclusive of seed coating and seed dressing.

As used herein, “subject” refers to a living organism having a central nervous system. In particular, subjects include, but are not limited to, human subjects or patients and companion animals. Exemplary companion animals may include domesticated mammals (e.g., dogs, cats, horses), mammals with significant commercial value (e.g., dairy cows, beef cattle, sporting animals), mammals with significant scientific values (e.g., captive or free specimens of endangered species), or mammals which otherwise have value. Suitable subjects also include: mice, rats, dogs, cats, ungulates such as cattle, swine, sheep, horses, and goats, lagomorphs such as rabbits and hares, other rodents, and primates such as monkeys, chimps, and apes. Subjects may be of any age including new born, adolescence, adult, middle age, or elderly.

As various changes could be made in the above compositions and methods without departing from the scope of the disclosure, it is intended that all matter contained in the above description and in the Examples given below, shall be interpreted as illustrative and not in a limiting sense.

1. A microbial-based composition, comprising:
a. a microorganism consortium in culture medium comprising at least three microorganisms, wherein said at least three microorganisms are co-cultured, wherein said at least three microorganisms are selected from the group consisting of:
i. at least one sulfide-utilizing microorganism;
ii. at least one species of lactic acid bacteria;
iii. at least one Bacillus species;
iv. Gluconacetobacter diazotrophicus; and
v. at least one yeast; and
b. at least one carbon source, wherein the at least one carbon source is selected from the group consisting of molasses, rum, and the combination thereof.

2. The microbial-based composition of claim 1, wherein the at least three microorganisms are selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma vixens.

3. (canceled)

4. (canceled)

5. (canceled)

6. The microbial-based composition of claim 1, further comprising an essential oil selected from the group consisting of Lippia javanica oil, anise oil, bay oil, bergamot oil, boronia oil, canola oil, carrot oil, cassia oil, catnip oil, cedarwood oil, chamomile oil, cinnamon oil, citronella oil, clary sage oil, clove oil, cypress oil, eucalyptus oil, galbanum oil, garlic oil, ginger oil, geranium oil, grapefruit oil, hazelnut oil, jasmine oil, jojoba oil, lavender oil, lavandin oil, lemon oil, lime oil, mandarin oil, nutmeg oil, orange oil, palma rosa oil, patchouli oil, Peru balsams, peppermint oil, rosemary oil, rosewood oil, sage oil, sandalwood oil, spear mint oil, star anise oil, tea tree oil, tangerine oil, thyme oil, tolu, verbena oil, white clover oil, and ylang ylang oil.

7. The microbial-based composition of claim 1, wherein said microorganism consortium in culture medium is fermented for about 24 hours to about 30 days.

8. (canceled)

9. A method of enhancing the health of a plant, comprising administering a therapeutic amount of a finished product to a recipient plant or seed, wherein the finished product comprises:
a. a microorganism consortium comprising Gluconacetobacter diazotrophicus and at least three microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma vixens, wherein the microorganisms are co-cultured;
b. at least one carbon source selected from the group consisting of rum, molasses, glucose, starch, cellulose, fructose, and sucrose; and
c. dechlorinated water.

10. The method of claim 9, wherein the microorganism consortium is fermented for at least twenty-four hours.

11. The method of claim 9, wherein the microorganism consortium is fermented no more than thirty days.

12. The method of claim 9, wherein the finished product further comprises an essential oil selected from the group consisting of Lippia javanica oil, anise oil, bay oil, bergamot oil, boronia oil, canola oil, carrot oil, cassia oil, catnip oil, cedarwood oil, chamomile oil, cinnamon oil, citronella oil, clary sage oil, clove oil, cypress oil, eucalyptus oil, galbanum oil, garlic oil, ginger oil, geranium oil, grapefruit oil, hazelnut oil, jasmine oil, jojoba oil, lavender oil, lavandin oil, lemon oil, lime oil, mandarin oil, nutmeg oil, orange oil, palma rosa oil, patchouli oil, Peru balsams, peppermint oil, rosemary oil, rosewood oil, sage oil, sandalwood oil, spear mint oil, star anise oil, tea tree oil, tangerine oil, thyme oil, tolu, verbena oil, white clover oil, and ylang ylang oil.

13. The method of claim 9, wherein the finished product is applied to a foliar surface of a plant.

14. The method of claim 9, wherein the finished product is applied to at least one area of plant growing medium surrounding at least one root of a plant.

15. The method of claim 9, wherein said plant is selected from the group consisting of cannabis sativa, cannabis indica, and cannabis ruderalis.

16. The method of claim 9, wherein said plant is selected from the group consisting of barley, corn, millet, oats, rice, rye, sorghum, and wheat.

17. A method for preserving silage quality and reducing damage to a cut plant product from at least one plant pathogen, comprising administering a therapeutic amount of a finished product to the at least one cut plant product, wherein the finished product comprises:
a. a microorganism consortium comprising Gluconacetobacter diazotrophicus and at least three microorganisms selected from the group consisting of Acetobacter ghanensis, Acetobacter pasteurianus, Bacillus subtilis, Bifidobacterium animalis, Bifidobacterium bifidum, Bifidobacterium longum, Enterococcus lactis, Enterococcus thermophilus, Lactobacillus acetotolerans, Lactobacillus acidophilus, Lactobacillus bulgaricus, Lactobacillus casei, Lactobacillus fermentum, Lactobacillus parafarraginis, Lactobacillus plantarum, Rhodopseudomonas palustris, Rhodopseudomonas sphaeroides, Saccharomyces cerevisiae, Pichia kudriavzevii, and Trichoderma vixens, wherein the microorganisms are co-cultured;
b. at least one carbon source selected from the group consisting of rum, molasses, glucose, starch, cellulose, fructose, and sucrose; and
c. dechlorinated water.

18. The method of claim 17, wherein said cut plant product is selected from the group comprising silage, oatlage, and haylage.

19. The method of claim 17, wherein said silage comprises at least one selection from the group consisting of corn, grain sorghum, Bermuda grass, star grass, limpograss, forage sorghum, pearl millet, ryegrass, alfalfa, red clover, hairy indigo, alyce clover, shamrock, vicia sativa, aeschynomene, and rhizoma perennial peanut.
